SEQUENCE LISTING (363 sequences)

SEQ ID NO: 1 - protein, 9 aa, Bos taurus
GFAGDDAPR

SEQ ID NO: 2 - protein, 11 aa, Bos taurus
DSYVGDEAQSK

SEQ ID NO: 3 - protein, 15 aa, Bos taurus
AGFAGDDAPRAVFPS

SEQ ID NO: 4 - protein, 16 aa, Bos taurus
GDDAPRAVFPSIVGRP

SEQ ID NO: 5 - protein, 15 aa, Bos taurus
YELPDGQVITIGNER

SEQ ID NO: 6 - protein, 16 aa, Bos taurus
SYELPDGQVITIGNER

SEQ ID NO: 7 - protein, 17 aa, Bos taurus
VGMGQKDSYVGDEAQSK

SEQ ID NO: 8 - protein, 16 aa, Bos taurus
YELPDGQVITIGNERF

SEQ ID NO: 9 - protein, 19 aa, Bos taurus
GFAGDDAPRAVFPSIVGRP

SEQ ID NO: 10 - protein, 17 aa, Bos taurus
SYELPDGQVITIGNERF

SEQ ID NO: 11 - protein, 20 aa, Bos taurus
LRVAPEEHPTLLTEAPLNPK

SEQ ID NO: 12 - protein, 22 aa, Bos taurus
HQGVMVGMGQKDSYVGDEAQSK

SEQ ID NO: 13 - protein, 19 aa, Molgula oculata
RVAPEEHPVLLTEAPLNPK

SEQ ID NO: 14 - protein, 21 aa, Homo sapiens
HQGVMVGMGQKDCYVGDEAQS

SEQ ID NO: 15 - protein, 16 aa, Seriola quinqueradiata
DNLEPCMYVCVVAQSK

SEQ ID NO: 16 - protein, 12 aa, Danio rerio
TPNVSVVDLTVR

SEQ ID NO: 17 - protein, 13 aa, Danio rerio
VVESTGVFTTIEK

SEQ ID NO: 18 - protein, 14 aa, Danio rerio
VPTPNVSVVDLTVR

SEQ ID NO: 19 - protein, 14 aa, Danio rerio
FRVPTPNVSVVDLT

SEQ ID NO: 20 - protein, 15 aa, Danio rerio
RVPTPNVSVVDLTVR

SEQ ID NO: 21 - protein, 10 aa, Tetronarce californica
GGDDLDPNYV

SEQ ID NO: 22 - protein, 10 aa, Tetronarce californica
SIDDLMPAQK

SEQ ID NO: 23 - protein, 14 aa, Tetronarce californica
DIIQTGVDNPGHPF

SEQ ID NO: 24 - protein, 15 aa, Tetronarce californica
DDIIQTGVDNPGHPF

SEQ ID NO: 25 - protein, 16 aa, Tetronarce californica
DDIIQTGVDNPGHPFI

SEQ ID NO: 26 - protein, 15 aa, Gallus gallus
DVIQTGVDNPGHPFI

SEQ ID NO: 27 - protein, 15 aa, Gallus gallus
DDVIQTGVDNPGHPF

SEQ ID NO: 28 - protein, 16 aa, Gallus gallus
TLDDVIQTGVDNPGHP

SEQ ID NO: 29 - protein, 16 aa, Gallus gallus
LDDVIQTGVDNPGHPF

SEQ ID NO: 30 - protein, 16 aa, Gallus gallus
DDVIQTGVDNPGHPFI

SEQ ID NO: 31 - protein, 17 aa, Gallus gallus
TLDDVIQTGVDNPGHPF

SEQ ID NO: 32 - protein, 10 aa, Oryctolagus cuniculus
SIDDMIPAQK

SEQ ID NO: 33 - protein, 10 aa, Bos taurus
ISGLIYEETR

SEQ ID NO: 34 - protein, 12 aa, Bos taurus
TVTAMDVVYALK

SEQ ID NO: 35 - protein, 12 aa, Bos taurus
DNIQGITKPAIR

SEQ ID NO: 36 - protein, 12 aa, Bos taurus
GLAGVENVTELK

SEQ ID NO: 37 - protein, 13 aa, Homo sapiens
VSLSLHADWAEPA

SEQ ID NO: 38 - protein, 12 aa, Arctogadus glacialis
TYAALSVLHSEK

SEQ ID NO: 39 - protein, 13 aa, Danio rerio
HVFGESDELIGQK

SEQ ID NO: 40 - protein, 7 aa, Rattus norvegicus
EEVNALT

SEQ ID NO: 41 - protein, 14 aa, Pagrus major
PGVTVKDVNQQEFV

SEQ ID NO: 42 - protein, 15 aa, Mus musculus
AVSEGTKAVTKYTSS

SEQ ID NO: 43 - protein, 16 aa, Mus musculus
HAVSEGTKAVTKYTSS

SEQ ID NO: 44 - protein, 17 aa, Homo sapiens
PDINVDVALTMPREILQ

SEQ ID NO: 45 - protein, 18 aa, Arctogadus glacialis
DIGAEALSRLVAVYPQTK

SEQ ID NO: 46 - protein, 12 aa, Gallus gallus
DVYVNDAFGTAH

SEQ ID NO: 47 - protein, 23 aa, Mus musculus
VERPEEGELSSGPAQLVGGVGQD

SEQ ID NO: 48 - protein, 21 aa, Bos taurus
AKVAVLGASGGIGQPLSLLLK

SEQ ID NO: 49 - protein, 17 aa, Bos taurus
VHDAILEDLVFPSEIVG

SEQ ID NO: 50 - protein, 17 aa, Homo sapiens
KQTAEMYVKADGSVEEA

SEQ ID NO: 51 - protein, 16 aa, Cyprinus carpio
VLNASVIPEGQFIDNK

SEQ ID NO: 52 - protein, 11 aa, Mus musculus
GRNKADNLDVP

SEQ ID NO: 53 - protein, 18 aa, Homo sapiens
NGQLAASVETAGDSELFL

SEQ ID NO: 54 - protein, 13 aa, Mus musculus
HLQLAVRNDEELN

SEQ ID NO: 55 - protein, 25 aa, Mus musculus
AVRNDEELNKLLGGVTIAQGGVLPN

SEQ ID NO: 56 - protein, 17 aa, Homo sapiens
TAGVPGFVLDTNASVSN

SEQ ID NO: 57 - protein, 22 aa, Alligator mississippiensis
AAVPSGASTGIYEALELRDNDK

SEQ ID NO: 58 - protein, 12 aa, Homo sapiens
VENMGRVNYGAY

SEQ ID NO: 59 - protein, 11 aa, Mus musculus
KKNEVATEECS

SEQ ID NO: 60 - protein, 9 aa, Homo sapiens
KSNGPNGNE

SEQ ID NO: 61 - protein, 10 aa, Tyto alba
INMQGQVVQT

SEQ ID NO: 62 - protein, 7 aa, Bos taurus
YDVAGQG

SEQ ID NO: 63 - protein, 21 aa, Mus musculus
KPCSSQCYDTKTPDPSVDVSL

SEQ ID NO: 64 - protein, 20 aa, Bos taurus
DEELDSLIKATIAGGGVIPH

SEQ ID NO: 65 - protein, 18 aa, Mus musculus
KLLGGVTIAQGGVLPNIQ

SEQ ID NO: 66 - protein, 17 aa, Mus musculus
LTAEILELAGNAARDNK

SEQ ID NO: 67 - protein, 22 aa, Mus musculus
LLGGVTIAQGGVLPNIQAVLLP

SEQ ID NO: 68 - protein, 23 aa, Mus musculus
LLGGVTIAQGGVLPNIQAVLLPK

SEQ ID NO: 69 - protein, 22 aa, Mus musculus
AVLEYLTAEILELAGNAARDNK

SEQ ID NO: 70 - protein, 26 aa, Mus musculus
RNDEELNKLLGGVTIAQGGVLPNIQA

SEQ ID NO: 71 - protein, 27 aa, Mus musculus
AVRNDEELNKLLGGVTIAQGGVLPNIQ

SEQ ID NO: 72 - protein, 28 aa, Mus musculus
AVRNDEELNKLLGGVTIAQGGVLPNIQA

SEQ ID NO: 73 - protein, 28 aa, Mus musculus
LAVRNDEELNKLLGGVTIAQGGVLPNIQ

SEQ ID NO: 74 - protein, 29 aa, Mus musculus
AVRNDEELNKLLGGVTIAQGGVLPNIQAV

SEQ ID NO: 75 - protein, 12 aa, Danio rerio
AVGKVIPELNGK

SEQ ID NO: 76 - protein, 12 aa, Danio rerio
RVPTPNVSVVDL

SEQ ID NO: 77 - protein, 13 aa, Danio rerio
RVPTPNVSVVDLT

SEQ ID NO: 78 - protein, 13 aa, Danio rerio
VVESTGVFTTIEK

SEQ ID NO: 79 - protein, 14 aa, Danio rerio
RVPTPNVSVVDLTV

SEQ ID NO: 80 - protein, 14 aa, Danio rerio
VPTPNVSVVDLTVR

SEQ ID NO: 81 - protein, 15 aa, Danio rerio
TTIEKASAHIKGGAK

SEQ ID NO: 82 - protein, 15 aa, Danio rerio
KAVGKVIPELNGKLT

SEQ ID NO: 83 - protein, 14 aa, Danio rerio
YVVESTGVFTTIEK

SEQ ID NO: 84 - protein, 15 aa, Danio rerio
RVIISAPSADAPMFV

SEQ ID NO: 85 - protein, 16 aa, Danio rerio
KAVGKVIPELNGKLTG

SEQ ID NO: 86 - protein, 18 aa, Danio rerio
DGRGASQNIIPASTGAAK

SEQ ID NO: 87 - protein, 17 aa, Danio rerio
RVIISAPSADAPMFVMG

SEQ ID NO: 88 - protein, 18 aa, Danio rerio
GVFTTIEKASAHIKGGAK

SEQ ID NO: 89 - protein, 18 aa, Danio rerio
SIFDAGAGIALNDHFVKL

SEQ ID NO: 90 - protein, 21 aa, Danio rerio
VIISAPSADAPMFVMGVNHEK

SEQ ID NO: 91 - protein, 11 aa, Xenopus laevis
VVSTDFNGDTH

SEQ ID NO: 92 - protein, 13 aa, Xenopus laevis
VVSTDFNGDTHSS

SEQ ID NO: 93 - protein, 15 aa, Xenopus laevis
KAVGKVIPELNGKIT

SEQ ID NO: 94 - protein, 16 aa, Xenopus laevis
KAVGKVIPELNGKITG

SEQ ID NO: 95 - protein, 16 aa, Bos taurus
KAVGKVIPDLKGKLTG

SEQ ID NO: 96 - protein, 15 aa, Oreochromis mossambicus
VLSGGTTMYPGIADR

SEQ ID NO: 97 - protein, 15 aa, Oreochromis mossambicus
YELPDGQVITIGNER

SEQ ID NO: 98 - protein, 16 aa, Oreochromis mossambicus
SYELPDGQVITIGNER

SEQ ID NO: 99 - protein, 18 aa, Oreochromis mossambicus
FAGDDAPRAVFPSIVGRP

SEQ ID NO: 100 - protein, 20 aa, Oreochromis mossambicus
AGFAGDDAPRAVFPSIVGRP

SEQ ID NO: 101 - protein, 21 aa, Oreochromis mossambicus
AGFAGDDAPRAVFPSIVGRPR

SEQ ID NO: 102 - protein, 21 aa, Oreochromis mossambicus
DLYANTVLSGGTTMYPGIADR

SEQ ID NO: 103 - protein, 20 aa, Oreochromis mossambicus
LRVAPEEHPVLLTEAPLNPK

SEQ ID NO: 104 - protein, 18 aa, Xenopus laevis
VAPEEHPTLLTEAPLNPK

SEQ ID NO: 105 - protein, 13 aa, Canis lupus familiaris
VIQTGVDNPGHPF

SEQ ID NO: 106 - protein, 14 aa, Canis lupus familiaris
DVIQTGVDNPGHPF

SEQ ID NO: 107 - protein, 17 aa, Canis lupus familiaris
TLDDVIQTGVDNPGHPF

SEQ ID NO: 108 - protein, 18 aa, Canis lupus familiaris
TLDDVIQTGVDNPGHPFI

SEQ ID NO: 109 - protein, 16 aa, Homo sapiens
TVDDVIQTGVDNPGHP

SEQ ID NO: 110 - protein, 13 aa, Cricetulus griseus
DLEPTVIDEVRTG

SEQ ID NO: 111 - protein, 15 aa, Cricetulus griseus
VDLEPTVIDEVRTGT

SEQ ID NO: 112 - protein, 16 aa, Cricetulus griseus
FVDLEPTVIDEVRTGT

SEQ ID NO: 113 - protein, 12 aa, Thunnus thynnus
GVKVLHGLDRAV

SEQ ID NO: 114 - protein, 13 aa, Thunnus thynnus
IAAHGVKVLHGLD

SEQ ID NO: 115 - protein, 13 aa, Thunnus thynnus
AAHGVKVLHGLDR

SEQ ID NO: 116 - protein, 14 aa, Thunnus thynnus
IAAHGVKVLHGLDR

SEQ ID NO: 117 - protein, 15 aa, Cyprinus carpio
DAVSKIDDLVGGLAA

SEQ ID NO: 118 - protein, 18 aa, Cyprinus carpio
AVGDAVSKIDDLVGGLAA

SEQ ID NO: 119 - protein, 19 aa, Cyprinus carpio
GAVGDAVSKIDDLVGGLAA

SEQ ID NO: 120 - protein, 12 aa, Homo sapiens
SRIIDTLDIGPA

SEQ ID NO: 121 - protein, 9 aa, Mus musculus
RLLLPGELA

SEQ ID NO: 122 - protein, 16 aa, Mus musculus
HAVSEGTKAVTKYTSS

SEQ ID NO: 123 - protein, 17 aa, Mus musculus
HAVSEGTKAVTKYTSSK

SEQ ID NO: 124 - protein, 19 aa, Thunnus albacares
AFPFLTPEQKKELSDIAHK

SEQ ID NO: 125 - protein, 12 aa, Bos taurus
TVTAMDVVYALK

SEQ ID NO: 126 - protein, 12 aa, Macruronus magellanicus
NIIHGSDTLENA

SEQ ID NO: 127 - protein, 9 aa, Bos taurus
WEQLDPSGR

SEQ ID NO: 128 - protein, 14 aa, Xenopus laevis
KKALAAGGYDVDKN

SEQ ID NO: 129 - protein, 9 aa, Oryzias latipes
LVVYPQTKT

SEQ ID NO: 130 - protein, 9 aa, Oryzias latipes
VVYPQTKTY

SEQ ID NO: 131 - protein, 10 aa, Oryzias latipes
LVVYPQTKTY

SEQ ID NO: 132 - protein, 16 aa, Oryzias latipes
IGADALGRMLVVYPQT

SEQ ID NO: 133 - protein, 17 aa, Oncorhynchus mykiss
KGTLVQTKGTGASGSFK

SEQ ID NO: 134 - protein, 21 aa, Oncorhynchus mykiss
SLVTKGTLVQTKGTGASGSFK

SEQ ID NO: 135 - protein, 15 aa, Cricetulus griseus
IGRIEDVTPIPSDST

SEQ ID NO: 136 - protein, 15 aa, Cyprinus carpio
DTGAPIRIPVGPETL

SEQ ID NO: 137 - protein, 18 aa, Cyprinus carpio
KVLDTGAPIRIPVGPETL

SEQ ID NO: 138 - protein, 19 aa, Bos taurus
HAEVRAGAVSALAKFGAQN

SEQ ID NO: 139 - protein, 12 aa, Homo sapiens
LAILENANVLAR

SEQ ID NO: 140 - protein, 14 aa, Homo sapiens
IVEPEILPDGDHDL

SEQ ID NO: 141 - protein, 13 aa, Mus musculus
RTVPPAVTGVTFL

SEQ ID NO: 142 - protein, 19 aa, Bos taurus
LTAEVLELAGNASKDLKVK

SEQ ID NO: 143 - protein, 25 aa, Bos taurus
RGDEELDSLIKATIAGGGVIPHIHK

SEQ ID NO: 144 - protein, 15 aa, Homo sapiens
KLASPMKPVPGTPPS

SEQ ID NO: 145 - protein, 15 aa, Salmo salar
AAVPSGASTGVHEAL

SEQ ID NO: 146 - protein, 22 aa, Homo sapiens
ENCFWIKVNENKYENVDLLCKL

SEQ ID NO: 147 - protein, 17 aa, Gymnothorax unicolor
YFSHWPETTPGSAPVKK

SEQ ID NO: 148 - protein, 14 aa, Sus scrofa
VPSPKVSDTVVEPY

SEQ ID NO: 149 - protein, 11 aa, Gallus gallus
IDYEQGGVLEV

SEQ ID NO: 150 - protein, 12 aa, Mus musculus
AETQYEEQIAKI

SEQ ID NO: 151 - protein, 16 aa, Xenopus laevis
FGAVGVGSSLTSFGAE

SEQ ID NO: 152 - protein, 16 aa, Bos taurus
AVVGVVAGGGRIDKPI

SEQ ID NO: 153 - protein, 19 aa, Homo sapiens
LNTLDQIADIVTSVFSSPV

SEQ ID NO: 154 - protein, 20 aa, Homo sapiens
RELSVRDVQATRVGLKDQRR

SEQ ID NO: 155 - protein, 12 aa, Homo sapiens
APATGGVKKPHR

SEQ ID NO: 156 - protein, 16 aa, Oryctolagus cuniculus
HPHTGEFTYKSSCTFN

SEQ ID NO: 157 - protein, 10 aa, Homo sapiens
KDISVLKGTL

SEQ ID NO: 158 - protein, 29 aa, Mus musculus
KNGGFFLRIHPDGRVDGVREKSDPHVKLQ

SEQ ID NO: 159 - protein, 23 aa, Mesocricetus auratus
KSTREAWTDGRQSTESETPTKSV

SEQ ID NO: 160 - protein, 24 aa, Xenopus laevis
QPGKTPKKAQNKSKPNQGHPPDET

SEQ ID NO: 161 - protein, 24 aa, Bos taurus
KDATGTLVGNVVHSRIINPLRLFV

SEQ ID NO: 162 - protein, 15 aa, Homo sapiens
PATTPAQPQQSQSQT

SEQ ID NO: 163 - protein, 31 aa, Danio rerio
LSNFIPDDEATLMKNGRYSCLVCSHRPVFDT

SEQ ID NO: 164 - protein, 23 aa, Bos taurus
ILCVRQICAVILESPPKGATIPY

SEQ ID NO: 165 - protein, 15 aa, Mus musculus
QHGRILDILLSTCSL

SEQ ID NO: 166 - protein, 19 aa, Homo sapiens
QKKLIAMLENGLGLPPGMW

SEQ ID NO: 167 - protein, 13 aa, Homo sapiens
INSSSSVVHKKDH

SEQ ID NO: 168 - protein, 20 aa, Homo sapiens
PQNRKEANPTPLTPGASSLS

SEQ ID NO: 169 - protein, 14 aa, Xenopus tropicalis
KMVDIQVSAKKMLD

SEQ ID NO: 170 - protein, 9 aa, Pygoscelis papua
ELGVGFATR

SEQ ID NO: 171 - protein, 13 aa, Pygoscelis papua
LGEEFDETTADDR

SEQ ID NO: 172 - protein, 13 aa, Homo sapiens
AWGPGLEGGVVGK

SEQ ID NO: 173 - protein, 12 aa, Homo sapiens
TFEPAEFIIDTR

SEQ ID NO: 174 - protein, 8 aa, Thunnus thynnus
VEWTQQER

SEQ ID NO: 175 - protein, 13 aa, Salmo salar
ALTDAETKAFLAD

SEQ ID NO: 176 - protein, 13 aa, Homo sapiens
TADLVSFTLIVGI

SEQ ID NO: 177 - protein, 15 aa, Danio rerio
VVLAYEPVWAIGTGK

SEQ ID NO: 178 - protein, 10 aa, Pseudochirops cupreus
VLTWIGAQPV

SEQ ID NO: 179 - protein, 9 aa, Homo sapiens
SDLADLRSC

SEQ ID NO: 180 - protein, 32 aa, Mus musculus
KADLLADHTEGIIKSILQGNAMLLRVLPAVYE

SEQ ID NO: 181 - protein, 12 aa, Homo sapiens
ARRATGNLSASC

SEQ ID NO: 182 - protein, 11 aa, Gallus gallus
KEPPNGNAFPP

SEQ ID NO: 183 - protein, 7 aa, Cavia porcellus
WCSRGCQ

SEQ ID NO: 184 - protein, 18 aa, Thunnus albacares
FPFLTPEQKKELSDIAHK

SEQ ID NO: 185 - protein, 11 aa, Mus musculus
ATTVPDSNSVP

SEQ ID NO: 186 - protein, 8 aa, Chrysochloris asiatica
IVAALTMV

SEQ ID NO: 187 - protein, 13 aa, Cavia porcellus
KWRYKLTLFAATF

SEQ ID NO: 188 - protein, 8 aa, Pongo abelii
ACSLLGHV

SEQ ID NO: 189 - protein, 13 aa, Stegastes partitus
GPPGPPGPPGPPG

SEQ ID NO: 190 - protein, 8 aa, Stegastes partitus
PAVPGPMG

SEQ ID NO: 191 - protein, 10 aa, Stegastes partitus
  MOD_RES (10)..(10): Oxidated Met
PAVPGPMGPX

SEQ ID NO: 192 - protein, 13 aa, Monopterus albus
GPLGPPGLPGPPG

SEQ ID NO: 193 - protein, 16 aa, Tauraco erythrolophus
GAPGPIGPPGPPGPPG

SEQ ID NO: 194 - protein, 13 aa, Tauraco erythrolophus
GPIGPPGPPGPPG

SEQ ID NO: 195 - protein, 14 aa, Tauraco erythrolophus
GPIGPPGPPGPPGA

SEQ ID NO: 196 - protein, 14 aa, Saccoglossus kowalevskii
GPPGPPGPPGPPGF

SEQ ID NO: 197 - protein, 17 aa, Copidosoma floridanum
AVGLRGSPGADGAIGPK

SEQ ID NO: 198 - protein, 10 aa, Copidosoma floridanum
GPLGPIGPLG

SEQ ID NO: 199 - protein, 13 aa, Branchiostoma belcheri
GLPGAPGLPGPPA

SEQ ID NO: 200 - protein, 12 aa, Branchiostoma belcheri
LPGAPGLPGPPA

SEQ ID NO: 201 - protein, 16 aa, Propithecus coquereli
GPAGIPGPIGPPGPPG

SEQ ID NO: 202 - protein, 13 aa, Propithecus coquereli
GIPGPIGPPGPPG

SEQ ID NO: 203 - protein, 17 aa, Charadrius vociferus
DCTYIPGIPGLPGSPGP

SEQ ID NO: 204 - protein, 8 aa, Camelus bactrianus
PGLPGPMG

SEQ ID NO: 205 - protein, 14 aa, Austrofundulus limnaeus
GLPGPPGLPGPAGP

SEQ ID NO: 206 - protein, 14 aa, Schistosoma mansoni
GLPGPLGPPGPPGM

SEQ ID NO: 207 - protein, 13 aa, Schistosoma mansoni
GLPGPLGPPGPPG

SEQ ID NO: 208 - protein, 10 aa, Sturnus vulgaris
PGGSGPVGPQ

SEQ ID NO: 209 - protein, 14 aa, Parus major
GLPGPLGPPGPGGP

SEQ ID NO: 210 - protein, 19 aa, Agrilus planipennis
  MOD_RES (3)..(3): Deamidated Gln
  MOD_RES (9)..(9): Deamidated Gln
PSXPGSGGXGGSGGLPGTP

SEQ ID NO: 211 - protein, 15 aa, Ovis aries
PGIKGLPGLPGPPGP

SEQ ID NO: 212 - protein, 13 aa, Sinocyclocheilus grahami
GLPGPPGPLGPDW

SEQ ID NO: 213 - protein, 12 aa, Hyalella azteca
GLRGPQGAPGEQ

SEQ ID NO: 214 - protein, 16 aa, Kryptolebias marmoratus
GPTGPMGPTGLPGPLG

SEQ ID NO: 215 - protein, 11 aa, Chlorocebus sabaeus
PSEAPAFPCST

SEQ ID NO: 216 - protein, 17 aa, Pogona vitticeps
GDPGPPGLPGLPGPSGP

SEQ ID NO: 217 - protein, 12 aa, Geospiza fortis
  MOD_RES (6)..(6): Oxidated Met
LGTLGXLGIEGI

SEQ ID NO: 218 - protein, 16 aa, Colius striatus
EKGEPGSPGDQGPTGP

SEQ ID NO: 219 - protein, 15 aa, Astyanax mexicanus
PQGPIGPPGPLGPPG

SEQ ID NO: 220 - protein, 13 aa, Astyanax mexicanus
GPIGPPGPLGPPG

SEQ ID NO: 221 - protein, 8 aa, Parus major
APGPVPMA

SEQ ID NO: 222 - protein, 8 aa, Saccoglossus kowalevskii
PGIRGPFG

SEQ ID NO: 223 - protein, 18 aa, Carlito syrichta
PLGPEGGPPGAPGPEGLP

SEQ ID NO: 224 - protein, 15 aa, Pseudomonas deceptionensis
  MOD_RES (11)..(11): Deamidated Asn
AHADVVQVQAXSLMR

SEQ ID NO: 225 - protein, 18 aa, Canis lupus familiaris
AEEAAAAGALLAARAGAM

SEQ ID NO: 226 - protein, 11 aa, Cynoglossus semilaevis
TGPLGPTGLPG

SEQ ID NO: 227 - protein, 17 aa, Chlorocebus sabaeus
PPTLVLLTLLVAAVLRC

SEQ ID NO: 228 - protein, 10 aa, Copidosoma floridanum
GPLGPIGPLG

SEQ ID NO: 229 - protein, 7 aa, Actinoplanes missouriensis
FPEDPFR

SEQ ID NO: 230 - protein, 8 aa, Oryza sativa
LIGGLGLI

SEQ ID NO: 231 - protein, 18 aa, Mus musculus
SGPPGKPGNVGLPGPSGP

SEQ ID NO: 232 - protein, 12 aa, Xenopus laevis
GPKGNDGNPGQP

SEQ ID NO: 233 - protein, 10 aa, Tupaia chinensis
PGPQGPPGYG

SEQ ID NO: 234 - protein, 11 aa, Trichogramma pretiosum
QSQSKGPDPSS

SEQ ID NO: 235 - protein, 10 aa, Ancylostoma ceylanicum
PDGNPGEEGQ

SEQ ID NO: 236 - protein, 17 aa, Nothobranchius furzeri
GPLGPSGLMGPPGPPGP

SEQ ID NO: 237 - protein, 9 aa, Clupea harengus
MHYVLWCLS

SEQ ID NO: 238 - protein, 13 aa, Branchiostoma belcheri
GLPGAPGLPGPPA

SEQ ID NO: 239 - protein, 16 aa, Echinococcus granulosus
  VARIANT (1)..(1): /replace=“pyro-Glu”
  SITE (1)..(16): /note=“Variant residues given in the sequence have no preference with respect to those in the annotations for variant positions”
QGRPGLPGPAGPKGSK

SEQ ID NO: 240 - protein, 14 aa, Ancylostoma ceylanicum
GGYGPGGGGGGFGP

SEQ ID NO: 241 - protein, 13 aa, Microplitis demolitor
NAGPTGPAGPTVG

SEQ ID NO: 242 - protein, 5 aa, Fundulus heteroclitus
YLLIQ

SEQ ID NO: 243 - protein, 15 aa, Orycteropus afer afer
GPPGEPGLPGPPGFI

SEQ ID NO: 244 - protein, 12 aa, Oryzias latipes
LPGVAGPLGLEG

SEQ ID NO: 245 - protein, 19 aa, Cynoglossus semilaevis
GLPGLPGPPGPPGPPGIPG

SEQ ID NO: 246 - protein, 13 aa, Ixodes ricinus
GDPGGPGLPGPLG

SEQ ID NO: 247 - protein, 13 aa, Methylobacterium brachiatum
PAQLPAGASAGDT

SEQ ID NO: 248 - protein, 15 aa, Ascaris suum
PGPDGKPGVNGMDGL

SEQ ID NO: 249 - protein, 16 aa, Ornithorhynchus anatinus
ERGLPGNPGAPGLPGN

SEQ ID NO: 250 - protein, 13 aa, Tupaia chinensis
  MOD_RES (7)..(7): Deamidated Gln
PGMPGLXGMPGNN

SEQ ID NO: 251 - protein, 9 aa, Callorhinchus milii
LNEMEYPYD

SEQ ID NO: 252 - protein, 14 aa, Ochotona princeps
PGERGDPGDTGVPG

SEQ ID NO: 253 - protein, 18 aa, Oikopleura dioica
PGPEGPPGLPGPPGPCGP

SEQ ID NO: 254 - protein, 18 aa, Melipona quadrifasciata
TPGTPPIPPQPGYPGSPG

SEQ ID NO: 255 - protein, 9 aa, Anas platyrhynchos
TSCLATSQH

SEQ ID NO: 256 - protein, 17 aa, Python bivittatus
GLRGLPGEPGPPGPSGP

SEQ ID NO: 257 - protein, 11 aa, Granulicella mallensis
FAPRVGFAYDL

SEQ ID NO: 258 - protein, 18 aa, Rhincodon typus
GDIGPPGPPGLPGESGPP

SEQ ID NO: 259 - protein, 6 aa, Lates calcarifer
GHRGYT

SEQ ID NO: 260 - protein, 12 aa, Thiothrix nivea
VNYGGDAGRPFG

SEQ ID NO: 261 - protein, 18 aa, Callorhinchus milii
GLPGPPGADGLPGPPGPA

SEQ ID NO: 262 - protein, 9 aa, Chlorocebus sabaeus
GPAGPSGPR

SEQ ID NO: 263 - protein, 18 aa, Elephantulus edwardii
GLPGFTGFPGAPGDPGHK

SEQ ID NO: 264 - protein, 6 aa, Fundulus heteroclitus
APDPFR

SEQ ID NO: 265 - protein, 15 aa, Polistes canadensis
IGLPGPQGPEGNKGP

SEQ ID NO: 266 - protein, 16 aa, Xenopus tropicalis
QGASGSPGIPGSPGVQ

SEQ ID NO: 267 - protein, 13 aa, Ancylostoma ceylanicum
RDGLPGIPGLEGE

SEQ ID NO: 268 - protein, 11 aa, Chlorocebus sabaeus
ASGPAGPSGPR

SEQ ID NO: 269 - protein, 8 aa, Bos taurus
SGPSSGLP

SEQ ID NO: 270 - protein, 9 aa, Copidosoma floridanum
  VARIANT (1)..(1): /replace=“pyro-Glu”
  SITE (1)..(9): /note=“Variant residues given in the sequence have no preference with respect to those in the annotations for variant positions”
QTIEPPRMF

SEQ ID NO: 271 - protein, 11 aa, Sinocyclocheilus rhinocerous
VDGFRGKPGAP

SEQ ID NO: 272 - protein, 13 aa, Oryctolagus cuniculus
GLPGLPGPPGPPG

SEQ ID NO: 273 - protein, 13 aa, Oryctolagus cuniculus
GPPGPPGPPGPPG

SEQ ID NO: 274 - protein, 12 aa, Oryctolagus cuniculus
  MOD_RES (11)..(11): Dioxidated Cys
GPPGPPGIPGXN

SEQ ID NO: 275 - protein, 12 aa, Fundulus heteroclitus
GPAGPSGPRGPA

SEQ ID NO: 276 - protein, 13 aa, Monopterus albus
GPLGPPGLPGPPG

SEQ ID NO: 277 - protein, 13 aa, Monopterus albus
GPPGPIGPLGPPG

SEQ ID NO: 278 - protein, 13 aa, Dipodomys ordii
GPIGPLGPPGPPG

SEQ ID NO: 279 - protein, 15 aa, Dipodomys ordii
PQGPIGPLGPPGPPG

SEQ ID NO: 280 - protein, 10 aa, Nothobranchius furzeri
  MOD_RES (10)..(10): Oxidated Met
PAVPGPMGPX

SEQ ID NO: 281 - protein, 16 aa, Tauraco erythrolophus
GAPGPIGPPGPPGPPG

SEQ ID NO: 282 - protein, 14 aa, Tauraco erythrolophus
GPIGPPGPPGPPGA

SEQ ID NO: 283 - protein, 14 aa, Branchiostoma belcheri
GPPGPPGIPGGPGP

SEQ ID NO: 284 - protein, 12 aa, Branchiostoma belcheri
LPGAPGLPGPPA

SEQ ID NO: 285 - protein, 13 aa, Echinococcus multilocularis
GPPGPIGPPGPPG

SEQ ID NO: 286 - protein, 14 aa, Tupaia chinensis
GIPGPIGPPGPPGL

SEQ ID NO: 287 - protein, 13 aa, Tupaia chinensis
GIPGPIGPPGPPG

SEQ ID NO: 288 - protein, 14 aa, Lates calcarifer
GPPGPPGLPGPPGA

SEQ ID NO: 289 - protein, 8 aa, Nanorana parkeri
GPLGYPGR

SEQ ID NO: 290 - protein, 14 aa, Oreochromis niloticus
GPIGPLGPPGPPGE

SEQ ID NO: 291 - protein, 15 aa, Callorhinchus milii
RAFSLNGNCSYLLLQ

SEQ ID NO: 292 - protein, 12 aa, Clupea harengus
  MOD_RES (5)..(5): Oxidated Met
GPPGXMGPPGME

SEQ ID NO: 293 - protein, 17 aa, Bactrocera latifrons
GPAGPPGEPGPIGPPGA

SEQ ID NO: 294 - protein, 16 aa, Atta colombica
PPGERGLPGLPGSPGP

SEQ ID NO: 295 - protein, 9 aa, Cricetulus griseus
AGFPGSPGY

SEQ ID NO: 296 - protein, 17 aa, Crocodylus porosus
PGMAGPLGPPGPPGPPG

SEQ ID NO: 297 - protein, 14 aa, Sinocyclocheilus grahami
GPIGPPGIPGPGGP

SEQ ID NO: 298 - protein, 18 aa, Geospiza fortis
  MOD_RES (13)..(13): Deamidated Gln
LGTLGMLGIEGIXGILGT

SEQ ID NO: 299 - protein, 17 aa, Pan troglodytes
GLPGPMGPPGLPGSPGP

SEQ ID NO: 300 - protein, 13 aa, Strongyloides ratti
GAKGEPAIGYDGA

SEQ ID NO: 301 - protein, 14 aa, Pediculus humanus corporis
GLPGPPGPIGPPGE

SEQ ID NO: 302 - protein, 15 aa, Xenopus laevis
  MOD_RES (11)..(11): Deamidated Gln
RDGLPGLRGDXGPLG

SEQ ID NO: 303 - protein, 25 aa, Plutella xylostella
  MOD_RES (9)..(9): Oxidated Met
IGAAGYPGXPGRLGARGPFGLKGER

SEQ ID NO: 304 - protein, 13 aa, Callorhinchus milii
GIPGLPGPPGPPG

SEQ ID NO: 305 - protein, 13 aa, Anser cygnoides domesticus
GLPGPIGPPGPPG

SEQ ID NO: 306 - protein, 14 aa, Anser cygnoides domesticus
GLPGPIGPPGPPGL

SEQ ID NO: 307 - protein, 16 aa, Oryzias latipes
  MOD_RES (9)..(9): Deamidated Gln
PLGRDASDXHIIEVVL

SEQ ID NO: 308 - protein, 13 aa, Sorex araneus
GPIGLPGPPGPPG

SEQ ID NO: 309 - protein, 19 aa, Ursus maritimus
  MOD_RES (9)..(9): Deamidated Gln
  MOD_RES (14)..(14): Deamidated Gln
GPMGPPGPXGPPGXGSPGP

SEQ ID NO: 310 - protein, 8 aa, Sorangium cellulosum
GIVMGTSH

SEQ ID NO: 311 - protein, 16 aa, Megasphaera sp.
LGADDKDAFGFDAGNG

SEQ ID NO: 312 - protein, 13 aa, Chrysemys picta bellii
PGNPGPMGPPGLP

SEQ ID NO: 313 - protein, 18 aa, Necator americanus
GRDGEKGYPGLPGEAGLP

SEQ ID NO: 314 - protein, 14 aa, Scleropages formosus
GLPGPPGPPGPMGP

SEQ ID NO: 315 - protein, 22 aa, Rousettus aegyptiacus
LPGLQGPPGFLGLPGLDGNMGL

SEQ ID NO: 316 - protein, 14 aa, Oryctolagus cuniculus
GPPGPPGPPGPPGE

SEQ ID NO: 317 - protein, 14 aa, Oryctolagus cuniculus
  VARIANT (1)..(1): /replace=“pyro-Glu”
  SITE (1)..(14): /note=“Variant residues given in the sequence have no preference with respect to those in the annotations for variant positions”
ERGPLGPPGLPGFT

SEQ ID NO: 318 - protein, 14 aa, Xenopus laevis
GPQGPAGPSGQPGL

SEQ ID NO: 319 - protein, 11 aa, Fundulus heteroclitus
VAIAVHSQDIT

SEQ ID NO: 320 - protein, 22 aa, Biomphalaria glabrata
PGSPGAAGERGLRGQTGMPGMP

SEQ ID NO: 321 - protein, 15 aa, Lasius niger
EPGLMGLPGVAGHVG

SEQ ID NO: 322 - protein, 10 aa, Nothobranchius furzeri
AGPAGIAGPR

SEQ ID NO: 323 - protein, 18 aa, Cyprinus carpio
PPGPTGSPGPIGPSGPPG

SEQ ID NO: 324 - protein, 13 aa, Poecilia reticulata
PIGGPGAPGVPGP

SEQ ID NO: 325 - protein, 20 aa, Homo sapiens
YPGEAGCPGEAGLPGAKGLT

SEQ ID NO: 326 - protein, 11 aa, Poecilia latipinna
LGPAGLPGPHQ

SEQ ID NO: 327 - protein, 18 aa, Rhincodon typus
GDIGPPGPPGLPGESGPP

SEQ ID NO: 328 - protein, 15 aa, Dendroctonus ponderosae
LIGPAGPPGPPGEPA

SEQ ID NO: 329 - protein, 16 aa, Esox lucius
LPGTVGLPGTIGPPGV

SEQ ID NO: 330 - protein, 12 aa, Anolis carolinensis
PGSPGPVGPPGM

SEQ ID NO: 331 - protein, 13 aa, Monopterus albus
GPIGPLGPPGLPG

SEQ ID NO: 332 - protein, 16 aa, Dipodomys ordii
GPLGPQGPIGPLGPPG

SEQ ID NO: 333 - protein, 8 aa, Ictalurus punctatus
GAIGERGE

SEQ ID NO: 334 - protein, 14 aa, Bemisia tabaci
YPGQPGTPGKPGQP

SEQ ID NO: 335 - protein, 13 aa, Pygoscelis adeliae
PAGPTGSQGLPGP

SEQ ID NO: 336 - protein, 12 aa, Trichoderma virens
PHSASASSVTAS

SEQ ID NO: 337 - protein, 9 aa, Scleropages formosus
AGPVGPMGP

SEQ ID NO: 338 - protein, 9 aa, Thamnophis sirtalis
RGMPGPPGT

SEQ ID NO: 339 - protein, 9 aa, Echinococcus multilocularis
GPAGDPGPQ

SEQ ID NO: 340 - protein, 21 aa, Xenopus laevis
PGLSGPKGEKGDFGPQGPPGT

SEQ ID NO: 341 - protein, 16 aa, Poeciliopsis prolifica
GASGQDGRDGIGPPGP

SEQ ID NO: 342 - protein, 15 aa, Condylura cristata
DKGEPGGPGADGAPG

SEQ ID NO: 343 - protein, 14 aa, Orcinus orca
GPIGPLGPPGLMGP

SEQ ID NO: 344 - protein, 13 aa, Caenorhabditis elegans
GPSGIPGLPGNPG

SEQ ID NO: 345 - protein, 13 aa, Bacillus wiedmannii
PGATGSQGIQGPQ

SEQ ID NO: 346 - protein, 14 aa, Lates calcarifer
TPGPKGEPGQPGFP

SEQ ID NO: 347 - protein, 20 aa, Caenorhabditis elegans
PAGYPGPRGAPGPVGPPGVA

SEQ ID NO: 348 - protein, 14 aa, Mycobacteroides abscessus
PGITGPAGPAGPLG

SEQ ID NO: 349 - protein, 8 aa, Aptenodytes forsteri
PVGMEYKE

SEQ ID NO: 350 - protein, 19 aa, Unknown
  source: /note=“Description of Unknown Mycobacterium phage Marie”
TGDTGPQGPKGDTGAQGPQ

SEQ ID NO: 351 - protein, 12 aa, Megachile rotundata
PGIPGAPGHPGA

SEQ ID NO: 352 - protein, 18 aa, Danio rerio
PAGPTGPPGPIGPSGPPG

SEQ ID NO: 353 - protein, 10 aa, Streptomyces silvensis
DGAESSLAGL

SEQ ID NO: 354 - protein, 14 aa, Kryptolebias marmoratus
GPAGGPGVDGYPGL

SEQ ID NO: 355 - protein, 14 aa, Sorex araneus
GPIGLPGPPGPPGF

SEQ ID NO: 356 - protein, 8 aa, Xiphophorus maculatus
  VARIANT (1)..(1): /replace=“pyro-Glu”
  SITE (1)..(8): /note=“Variant residues given in the sequence have no preference with respect to those in the annotations for variant positions”
ERGLPGFI

SEQ ID NO: 357 - protein, 13 aa, Tauraco erythrolophus
GPIGPPGPPGPPG

SEQ ID NO: 358 - protein, 13 aa, Crocodylus porosus
GPLGPPGPPGPPG

SEQ ID NO: 359 - protein, 21 aa, Geospiza fortis
GILGTLGMLGIEGIQGILGTL

SEQ ID NO: 360 - protein, 11 aa, Callorhinchus milii
SNADQRSIEGT

SEQ ID NO: 361 - protein, 16 aa, Callorhinchus milii
PGQPGLPGLPGPPGAP

SEQ ID NO: 362 - protein, 13 aa, Oryctolagus cuniculus
  VARIANT (1)..(1): /replace=“pyro-Glu”
  SITE (1)..(13): /note=“Variant residues given in the sequence have no preference with respect to those in the annotations for variant positions”
ERGPLGPPGLPGF

SEQ ID NO: 363 - protein, 13 aa, Saccoglossus kowalevskii
GPPGPPGPNGPPG

(19) United States
(12) Patent Application Publication
(10) Pub. No.: US 2023/0389573 A1
(43) Pub. Date: Dec. 7, 2023

(54) Antihypertensive Food Ingredients for Companion Animal Applications

(71) Applicant: TEAGASC - The Agriculture and Food Development Authority, Carlow (IE)

(72) Inventors: Maria Hayes, Dublin (IE); Ivan Milovanovic, Dublin (IE); Caoimhe Gargan, Dublin (IE)

(21) Appl. No.: 18/250,822

(22) PCT Filed: Oct. 27, 2021
(86) PCT No.: PCT/EP2021/079822

§ 371 Date: Apr. 27, 2023

(30) Foreign Application Priority Data
Oct. 27, 2020 (EP) 20204217.2

(51) Int. Cl. A23K 20/147; A23K 10/22; A23K 10/24; A23K 10/26; A23K 50/42; A61K 38/17; A61P 9/12

(52) U.S. Cl. CPC A23K 20/147; A23K 10/22; A23K 10/24; A23K 10/26; A23K 50/42; A61K 38/1706; A61P 9/12

(57) ABSTRACT

A marine peptide product derived from by-products of fish processing is provided. In particular, an animal food product, such as a biscuit, comprising marine peptides is provided. This product has health benefits including antihypertension activities.

### FIELD OF THE INVENTION

The current invention relates to a marine peptide product derived from by-products of fish processing. In particular, the invention relates to an animal food product comprising marine peptides, which has health benefits including antihypertension activities.

### BACKGROUND TO THE INVENTION

Many pet owners are concerned with both the nutritional and health needs of their pets. To meet nutritional requirements, pet food formulas can be selectively altered to vary the protein content, vitamin and mineral content and the caloric content. The balance of these ingredients becomes critical when making a dry pet food because a portion of the formula must be flours, which are required for structure. When making a dog/cat biscuit, the requirement that a portion of the formula be for structural ingredients becomes even more critical. The alternation of nutritional ingredients can affect the colour, biscuit strength, rise of the biscuit and the hardness of the biscuit. It is often desirable that the texture of dog biscuits intended for older or “senior” dogs provides a softer chew.

Common dog biscuits typically contain between 60% and 70% wheat flour, 10% soybean meal, 6% percent meat and bone meal and between 2% and 3% fat in the form of tallow. 11-12% miscellaneous ingredients are added. These biscuits typically contain about 1.5% fiber and about 20-22% protein. The majority of the protein is derived from the wheat flours used to make the dog/cat biscuit. The substitution of a portion of wheat flour with another ingredient can cause difficulties in the manufacturing of dog biscuits so that texture and appearance of the final product is affected.

Angiotensin-converting enzyme-I (ACE-1) inhibitors represent one of the most commonly used categories of drugs in canine and feline medicine. ACE-1 inhibitors currently approved for use in veterinary medicine are the pro-drugs benazepril, enalapril, imidapril and ramipril and are generally well tolerated. Benazepril, enalapril, imidapril and ramipril are approved for dogs with chronic heart failure (CHF), especially in those with chronic valvular disease. In such clinical settings, ACE-1 inhibitors improve hemodynamics and clinical signs and increase survival time. There have been reports of some benefit in cats with hypertrophic cardiomyopathy in two non-controlled investigations. Mitral valve regurgitation (MR) secondary to degeneration of the mitral valve apparatus is the most common cardiac disease in dogs, and the incidence of MR is approximately 30% in dogs aged 13 years and older. MR is a progressive disease that in severe instances can result in death, despite medical treatment. The progressive character of MR is intimately related to the renin-angiotensin aldosterone system (RAAS) that regulates blood pressure (BP), tissue perfusion, and fluid balance. Like the other cardiac disorders, the dysregulation of RAAS is a key to the process of chronic heart failure (CHF) in dogs with MR. Pharmacological inhibition of the RAAS with angiotensin-converting enzyme (ACE-1) inhibitors plays an important role in the management of MR, as indicated by many reports describing how the long-term efficacy of inhibiting ACE resulted in an improvement of congestive heart failure in dogs with MR.

Renal diseases, especially chronic renal failure (CRF), are common in canine and feline medicine. The renin-angiotensin-aldosterone system (RAAS) plays a pivotal role in these conditions in the development of renal lesions and the progression of kidney dysfunction. Angiotensin-converting enzyme inhibitors (ACEI) are currently considered as the most efficient agents in therapeutic strategies. The benefit of an ACEI treatment can be explained by at least three mechanisms: ACEI limit systemic and glomerular capillary hypertension, have an antiproteinuric effect, and retard the development of glomerulosclerosis and tubulointerstitial lesions. These effects have been studied in dogs and cats, and there is some evidence to support the recommendation of ACEI therapy in dogs and cats with CRF. Nevertheless, the prescription of ACE-1 in such patients should take into account the potential influence of renal impairment on ACE-1 disposition, and adverse effects on the renal function itself (especially hypotension and acute reductions in glomerular filtration rate). The risk of drug interaction with diuretics, nonsteroidal anti-inflammatory drugs, and anaesthetics, should not be overestimated. Furthermore, hypotension may occur in patients on a low sodium diet.

Diet may be also considered as a factor for reducing the risk to the heart of dogs and cats and indeed humans. Although antihypertensive drugs are available on the market, nutritionists claim that peptides which lower BP found in food are safer than “traditional” drugs and can be used as preventive agents in human and indeed animal heart health.

Functional foods provide health benefits if they are consumed on a regular basis as part of a varied diet. Functional foods for pets can modify gastrointestinal physiology, promote changes in biochemical parameters, improve brain functions and may reduce or minimize the risk of developing specific pathologies. This evidence derives largely from clinical studies while only limited evidence is available from studies in dogs and cats.

Pelagic fish species, including mackerel (Scomber scombrus), Blue whiting (Micromesistius poutassou), Horse mackerel (Scad, Trachurus trachurus) and Atlantic herring (Clupea harengus) are in the top 10 species contributing to global total fish catch and are valued for their n−3 long chain polyunsaturated fatty acids (PUFAs), vitamin D and protein content (Lindqvist et al., 2007).

During fish processing, by-products can account for up to 75% of the total catch by weight. Blood is a major by-product of fish processing and can originate from storage on boats, boat unloading, butchering, and processing, scaling, filleting, skinning, and evisceration. This blood-water poses an environmental and economic cost, and it can lead to contamination of the marine environment by raising the biological oxygen demand (BOD) which may cause algae blooms (Islam et al., 2004).

Several problems exist concerning utilisation of fish blood. Firstly, collection and drying of fish blood is difficult as the properties of fish blood vary from warm blooded animals. It is difficult to separate fish blood from the process water after bleeding and process water can pollute the fish blood with materials including faeces, salt and fish scales. Where fish blood is collected along with process waters the composition of these “blood-waters” can vary depending on the species caught, the volume of fish processed, the processing techniques employed (i.e., hand filleting V's mechanical) and other parameters including the temperature on board and the volume of water used during processing.

Skin and bone, which are known to be rich in collagen, make up approximately 30% of the weight of by-products from blue whiting surimi manufacture. Blue Whiting (Micromesistius poutassou) is a pelagic fish found extensively in the northern hemisphere.

CN105063150 discloses a method of preparing peptides from tuna derived blood, by hydrolysing the fish blood with alkaline protease and passing the hydrolysate through an ultrafiltration unit having a cut-off of 10 KDa.

Huang Chun-Yung et al., (Marine Drugs, vol. 16, no. 10, 2018) discloses a method for producing peptides comprising extraction of gelatin from milkfish scales, hydrolysis to produce a hydrolysate and subsequent separation by passing the hydrolysate through an ultrafiltration membrane with a cut-off of 3 KDa.

Khiari Zied et al., (Journal of The Science of Food and Agriculture, vo. 94, no. 8, December 2013) discloses a method for producing peptides comprising extraction of fish gelatin from mackerel skin, preparation of a hydrolysate and isolation by passing via an ultrafiltration filter with a cut-off of 3 kDa.

US2018/352832 discloses preparation of a protein hydrolysate from Calanus finmarchicus comprising peptides of 1-6 KDa, peptides of 0.2-1 KDa and amino acids <200 Da. The product is rich in taurine and arginine. The authors speculate high palatability when used as feed for cats and dogs.

Chen Junde et al., (Marine Drugs, vol. 16, no. 7, 2018) discloses the extraction of gelatin from lizardfish and obtaining peptides from the extracted gelation. The hydrolysate was produced by using a pepsin enzyme. Fractionation of the gelatin hydrolysate was carried out by chromatography.

The current invention serves to alleviate the problems associated with the prior art. The current inventors provide a method to recover, stabilise and characterise by-products of fish processing, such as blood-water collected during processing of pelagic fish and skin and bone from surimi manufacture and to provide an improved functional food in pet nutrition comprising extracts from fish processing. This functional food has a use in pet nutrition for disease prevention and is also appetizing and appealing in terms of appearance.

### SUMMARY OF THE INVENTION

The current invention relates to animal food products (herein referred to as “the food product of the invention”). Most specifically, the food product is an animal biscuit, particularly for dogs and cats. The food product of the invention comprises peptides derived from marine by-products generated during the processing of fish. The by-products include one or more of fish skins, bone, gut waste and bloodwaters.

The peptides are obtained by recovering the by-product using filtration methods with 3 kDa and/or 10 kDa membranes. Each fraction, or “marine peptide product”, has a particular peptide and amino acid composition and the fraction is used to make the food product of the invention.

Most advantageously, the animal food product of the invention has health benefits, as well as a desirable appearance and texture. The use of the by-product extract with farinaceous ingredients provides a food product in which the overall protein content is increased in essential amino acids when compared to standard animal biscuits and has ACE-1 inhibitory activity. This invention proposes the use of peptide ACE-1 inhibitors for prevention of disease through diet, rather than cure using pharmaceutical drugs. Heart health animal biscuits do not currently exist on the market. Most advantageously, the action of the ACE-1 inhibitory peptides remains active when incorporated into the biscuit.

The current inventors have shown ACE-1 inhibitory activity of peptide product from blue whiting and mackerel blood water in FIG. 6. Renin inhibition is illustrated in FIG. 7. ACE-1 inhibition of gelatine hydrolysate peptide product is shown in FIG. 14. Notably, FIG. 22 illustrates the ability of the peptide product of the invention to reduce hypertension in dogs via ACE-1 inhibition in vivo.

The peptides of the current invention have not been identified previously from fish species. Furthermore, the inventors are the first to show their use for ACE-1 inhibition and the specific use for the prevention of hypertension in animals, especially companion animals.

In one aspect, there is provided a marine peptide product.

In an embodiment, the marine peptide product is derived or obtained from the by-products of fish processing. Preferably, the by-products include one or more of fish skins, bone, gut waste and bloodwaters.

Preferably, the fish by product used in the current invention is blue whiting or mackerel by product. This may be fish skin, bone, gut waste and/or bloodwaters.

In an embodiment, the fish by-product is bloodwater derived from pelagic or whitefish processing. Preferably, the by-product is pelagic fish bloodwater. Still preferred, the by product is bloodwater from blue whiting or mackerel.

In an embodiment, the fish by product is fish skins, bone, gut waste and bloodwaters. The fish from which it derives can be any fish, such as any fish source from which gelatine and/or gelatine hydrolysed peptides can be obtained. Examples include but are not limited to mackerel, horse mackerel, blue whiting, whitefish and shellfish. In this embodiment, gelatine/collagen is extracted from the fish by product. Preferably, the fish is pelagic fish, such as blue whiting.

In an embodiment, the marine peptide product comprises one or more peptides comprising (or consisting of) a sequence selected from SEQUENCE ID NO. 1 to SEQUENCE ID NO. 188, or a variant thereof.

In an embodiment, the marine peptide product comprises one or more peptides comprising (or consisting of) a sequence selected from SEQUENCE ID NO. 1 to SEQUENCE ID NO. 64, or a variant thereof.

In an embodiment, the marine peptide product comprises one or more peptides comprising (or consisting of) a sequence selected from SEQUENCE ID NO. 65 to SEQUENCE ID NO. 169, or a variant thereof.

In an embodiment, the marine peptide product comprises one or more peptides comprising (or consisting of) a sequence selected from SEQUENCE ID NO. 170 to SEQUENCE ID NO. 188, or a variant thereof.

In an embodiment, the marine peptide product comprises one or more peptides comprising (or consisting of) a sequence selected from SEQUENCE ID NO. 189 to SEQUENCE ID NO. 271, or 363 or a variant thereof.

In an embodiment, the marine peptide product comprises one or more peptides comprising (or consisting of) a sequence selected from SEQUENCE ID NO. 272 to SEQUENCE ID NO. 362, or a variant thereof.

Preferably, the marine peptide product may further comprise at least one fatty acid. The fatty acids may be one or more of the fatty acids listed in Table 5. The fatty acid may be all of the fatty acids listed in Table 5.

In an embodiment, the marine protein product is a hydrolysed marine protein product. i.e. a hydrolysate.

Typically, the pelagic fish is blue whiting or mackerel.

The marine peptide protein may be in the form of a powder, such as a dried protein powder.

In one aspect, the invention provides an animal food product, or comestible product, comprising a marine peptide product. The marine protein product is the marine protein product of the invention as described herein.

Preferably, the animal is a companion animal, such as a dog or a cat.

Preferably, the food product is a biscuit. The food product may be an extruded snack, a dried powder, a kibble, a bounce ball, or energy ball, or any similar suitable animal food product. Typically, the biscuit is suitable for consumption by a dog and/or a cat.

Typically, the biscuit further comprises (or consists of) one or more of wheat flours, water, oil, typically vegetable oil.

In an aspect, there is provided a peptide having a sequence selected from SEQUENCE ID NO 1 to SEQUENCE ID NO. 363, or a variant of SEQUENCE ID NO 1 to SEQUENCE ID NO. 363 (“peptide of the invention”).

A further aspect of the invention provides a method for producing a marine peptide product from a marine by-product, the method comprising the following steps:
filtering a marine by-product using one or more filters selected from 3 kDa filter membrane and 10 kDa filter membrane, to obtain at least one retentate, a 3 kDa fraction and/or a 10 kDa fraction.

The marine peptide product is provided in the obtained fractions.

In an embodiment, the method further comprises a step of drying the retentate, 10 kDa fraction and/or 3 kDa fraction. Any suitable drying method may be used. Preferably, the drying method is selected from the group comprising spray drying, freeze drying and pulse combustion.

Alternatively, the retentate or fraction may be provided in the form of a paste or a liquid product. Any suitable known method of providing a paste or liquid product may be used.

In an embodiment, the by-product is bloodwaters from pelagic fish.

In an embodiment, prior to filtering, the method comprises an optional step of extracting gelatine from the by-product to provide a by-product in the form of a gelatine extract. In this embodiment, the method further comprises a step of hydrolysing the gelatine extract with one or more hydrolyses enzymes. In this embodiment, it is the hydrolysed gelatine extract that is the by-product that is then filtered using one or more filters as per the above-described method.

Prior to extracting the gelatine, the by-product may be pre-treated. This pre-treatment step may comprise one or more acid treatment steps. The acid may be selected from the group comprising NaOH, HCL, sulphuric acid, citric acid and acetic acid.

This may be pre-treatment with an amount of NaOH, following by an amount of sulphuric acid, followed by an amount of acetic acid. The product may be washed with water in between each step of addition. The product may be washed after the final step.

This may be pre-treatment with an amount of NaOH, following by an amount of HCL following by an amount of acetic acid. The product may be washed with water in between each step of addition. The product may be washed after the final step.

This may be a pre-treatment with an amount of an enzyme following by an amount of HCL. The enzyme may be alcalase. The product may be washed with water in between each step of addition. The product may be washed after the final step.

This may be pre-treatment with an amount of NaOH, following by an amount of citric acid. The product may be washed with water in between each step of addition. The product may be washed after the final step.

After the pre-treatment step the gelatine is extracted, for example overnight in a shaker, preferably, using water (3:1 v/w to material) at 45° C. in a MaxQ 8000 laboratory shaker (Thermo Fisher Scientific, MA USA) at 200 rpm speed.

More specifically, four standard operating procedures (SOP) for pre-treatment were assessed by the inventors, three of which (SOP1, SOP2 and SOP4) included chemical treatment, while SOP3 consisted of a combination of both chemical and enzymatic pre-treatment.

SOP1—The skin and bone material was washed once using tap water (6:1 v/w to material) and pre-treated three times in consecutive order as follows with 0.2% (w/v) NaOH (6:1 v/w skin and bone) for 40 min followed by washing with water, then with 0.2% (v/v) sulphuric acid (6:1 v/w to skin and bone) for 40 min followed by washing with water, and finally using 0.05M acetic acid (6:1 v/w to skin and bone) for 40 min followed by washing with water.

SOP2—). The skin and bone material was washed once using tap water (6:1 v/w to material) and pre-treated three times with 0.1M NaOH (6:1 v/w to material) for 30 min. After washing with water, it was treated with 0.25M HCl (6:1 v/w to material) for 2 h, and then with 0.05M acetic acid (6:1 v/w to material) for 1 h, followed by washing with water.

SOP3—The skin and bone material was washed once using tap water (6:1 v/w to material) and hydrolysed with alcalase enzyme (Sigma Aldrich, Ireland; 2.5 U/ml) in 0.1M K-phosphate buffer (pH=8; 3:1 v/w buffer to material+0.1% (w/w to material) enzyme) for 1.5 h at 40° C. After heating the mixture to boiling for 5 min to inactivate the enzyme and washing with water, the material was then treated with 0.25M HCl (6:1 v/w to material) for 2 h and washed with water.

SOP4—The skin and bone material was washed once using tap water (6:1 v/w to material) and pre-treated three times with 0.1M NaOH (6:1 v/w to material) for 30 min. After washing with water, it was treated with 1M citric acid (6:1 v/w to material) for 2 h and finally washed with water.

After pre-treatment using the individual SOPs the by-product skin and bone material was washed with water until neutral (pH 6-7) and gelatine was extracted overnight using water (3:1 v/w to material) at 45° C. in a MaxQ 8000 laboratory shaker (Thermo Fisher Scientific, MA USA) at 200 rpm speed. Following gelatine extraction, solids were removed by filtration through Whatman No 4 filter paper using a BQchi funnel. The clear extracts were then freeze dried (FD80GP, Cuddon Freeze Dry, New Zealand) using the following program: initial temperature −20° C.; 5 steps; run time 90 h; end temperature 0° C. Dried material was placed in sterile sealed 250 ml containers and stored at −80° C. until analyses.

The composition of the extracted gelatine may be: 348 (γ-chain), 215 (β-chain), 117 (α2-chain), or a gelatine as disclosed herein.

The by-product from which gelatine is extracted is one or more of fish skins, bone and gut waste. The fish is any fish. Examples include but are not limited to pelagic fish and white fish. Typically, the by-product is skin and/or bone derived from processing of blue whiting into surimi products.

The enzyme may be papain, alcalase, protamex flavourzyme, or a suitable proteolytic enzyme or similar enzymes.

A further aspect provides a marine peptide product obtained by the method of the invention. The marine peptide product may be the 3 kDa fraction or a 10 kDa fraction.

A still further aspect provides a food product, or comestible product, comprising the marine peptide product obtained by the method of the invention.

An aspect of the invention provides a marine peptide product, the peptide, or food product of the invention, for use as a medicament.

An aspect of the invention provides a marine peptide product, the peptide, or food product of the invention, for use in a method of treatment or prevention of disease in an animal.

An aspect of the invention provides a marine peptide product, the peptide, or food product of the invention, for use in a method of prevention of disease or condition in an animal.

In an embodiment, the disease selected form the group comprising heart disease and renal disease or an inflammatory disease or condition.

The condition may be high blood pressure or hypertension.

In an embodiment, the marine peptide product or food product of the invention, may be for use in improving or maintaining eye health, or prevention or treatment of eye disease.

The heart disease may be selected from the group comprising, but not limited to, chronic heart failure (CHF), chronic valvular disease, hypertrophic cardiomyopathy, mitral valve regurgitation (MR), e.g., secondary to degeneration of the mitral valve apparatus.

In an embodiment, the marine peptide product or food product of the invention, may be for use to prevent high blood pressure or hypertension.

In an embodiment, the disease or condition is one associate with a metabolic syndrome, for examples, heart health or congestive heart failure.

The renal disease may be selected from the group comprising but not limited to chronic renal failure (CRF).

A further aspect of the invention provides a method of treatment or prevention of a disease or condition in an animal. The disease or conditions are those described herein for the use of the invention.

A further aspect of the invention provides a method of extracting gelatine as disclosed herein.

### Definitions

All publications, patents, patent applications and other references mentioned herein are hereby incorporated by reference in their entireties for all purposes as if each individual publication, patent or patent application were specifically and individually indicated to be incorporated by reference and the content thereof recited in full.

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the reduction in accumulation of pathological levels of lysosomal enzymes). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

The term “peptide” used herein refers to a polymer composed of 2 to 50 amino acid monomers typically via peptide bond linkage. Peptides (including fragments and variants thereof) of and for use in the invention may be generated wholly or partly by chemical synthesis or by expression from nucleic acid or may be obtained from a marine by-product. For example, the peptides of and for use in the present invention can be readily prepared according to well-established, standard liquid or, preferably, solid-phase peptide synthesis methods known in the art (see, for example, J. M. Stewart and J. D. Young, Solid Phase Peptide Synthesis, 2nd edition, Pierce Chemical Company, Rockford, Illinois (1984), in M. Bodanzsky and A. Bodanzsky, The Practice of Peptide Synthesis, Springer Verlag, New York (1984). When necessary, any of the peptides employed in the invention can be chemically modified to increase their stability. A chemically modified peptide or a peptide analog includes any functional chemical equivalent of the peptide characterized by its increased stability and/or efficacy in vivo or in vitro in respect of the practice of the invention.

The term peptide analog also refers to any amino acid derivative of a peptide as described herein. A peptide analog can be produced by procedures that include, but are not limited to, modifications to side chains, incorporation of unnatural amino acids and/or their derivatives during peptide synthesis and the use of cross-linkers and other methods that impose conformational constraint on the peptides or their analogs. Examples of side chain modifications include modification of amino groups, such as by reductive alkylation by reaction with an aldehyde followed by reduction with NaBH₄; amidation with methylacetimidate; acetylation with acetic anhydride; carbamylation of amino groups with cyanate; trinitrobenzylation of amino groups with 2, 4, 6, trinitrobenzene sulfonic acid (TNBS); alkylation of amino groups with succinic anhydride and tetrahydrophthalic anhydride; and pyridoxylation of lysine with pyridoxa-5′-phosphate followed by reduction with NABH₄. The guanidino group of arginine residues may be modified by the formation of heterocyclic condensation products with reagents such as 2,3-butanedione, phenylglyoxal and glyoxal. The carboxyl group may be modified by carbodiimide activation via o-acylisourea formation followed by subsequent derivatization, for example, to a corresponding amide. Sulfhydryl groups may be modified by methods, such as carboxymethylation with iodoacetic acid or iodoacetamide; performic acid oxidation to cysteic acid; formation of mixed disulphides with other thiol compounds; reaction with maleimide; maleic anhydride or other substituted maleimide; formation of mercurial derivatives using 4-chloromercuribenzoate, 4-chloromercuriphenylsulfonic acid, phenylmercury chloride, 2-chloromercuric-4-nitrophenol and other mercurials; carbamylation with cyanate at alkaline pH. Tryptophan residues may be modified by, for example, oxidation with N-bromosuccinimide or alkylation of the indole ring with 2-hydroxy-5-nitrobenzyl bromide or sulphonyl halides. Tryosine residues may be altered by nitration with tetranitromethane to form a 3-nitrotyrosine derivative. Modification of the imidazole ring of a histidine residue may be accomplished by alkylation with iodoacetic acid derivatives or N-carbethoxylation with diethylpyrocarbonate. Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Peptide structure modification includes the generation of retro-inverso peptides comprising the reversed sequence encoded by D-amino acids.

The term “modified peptide” is used interchangeably with the term derivative of the peptide. The modified peptide includes a peptide which has been substituted with one or more groups as defined herein. The modification may be any modified that provides the peptides and or the composition of the invention with an increased ability to penetrate a cell. The modification may be any modification that increases the half-life of the composition or peptides of the invention. In one embodiment, the group is a protecting group. The protecting group may be an N-terminal protecting group, a C-terminal protecting group or a side-chain protecting group. The peptide may have one or more of these protecting groups. The person skilled in the art is aware of suitable techniques to react amino acids with these protecting groups. These groups can be added by preparation methods known in the art, for example the methods as outlined in paragraphs [0104] to [0107] of US2014120141. The groups may remain on the peptide or may be removed. The protecting group may be added during synthesis. In an embodiment of the invention the peptides may be substituted with a group selected from one or more straight chain or branched chain, long or short chain, saturated, or unsaturated, substituted with a hydroxyl, amino, amino acyl, sulfate or sulphide group or unsubstituted having from 1 to 29 carbon atoms. N-acyl derivatives include acyl groups derived from acetic acid, capric acid, lauric acid, myristic acid, octanoic acid, palmitic acid, stearic acid, behenic acid, linoleic acid, linolenic acid, lipoic acid, oleic acid, isosteric acid, elaidoic acid, 2-ethylhexaneic acid, coconut oil fatty acid, tallow fatty acid, hardened tallow fatty acid, palm kernel fatty acid, lanolin fatty acid or similar acids. These may be substituted or unsubstituted. When substituted they are preferably substituted with hydroxyl, or sulphur containing groups such as but not limited to SO₃H, SH, or S—S. In an embodiment of the current invention, the peptide is R₁—X— R₂. R₁ and/or R₂ groups respectively bound to the amino-terminal (N-terminal) and carboxyl-terminal (C-terminal) of the peptide sequence. In one embodiment, the peptide is R₁—X. Alternatively, the peptide is X— R₂. Preferably, R₁ is H, C₁₋₄ alkyl, acetyl, benzoyl or trifluoroacetyl; X is the peptide of the invention; R₂ is OH or NH₂. In an embodiment, R₁ is selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, Tert-butyloxycarbonyl, 9-fluorenylmethyloxycarbonyl (Fmoc) and R₅—CO—, wherein R₅ is selected from the group formed by H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl; R₂ is selected from the group formed by —NR₃R₄, —OR₃ and —SR₃, wherein R₃ and R₄ are independently selected from the group formed by H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl; and with the condition that R₁ and R₂ are not α-amino acids.

“Fragment” means a segment of a protein selected from SEQUENCE ID NO's, the fragment typically being contiguous amino acids in length.

“C-terminal domain” as applied to a fragment means the first three amino acids at the c-terminus of the fragment.

“N-terminal domain” as applied to a fragment means the last three amino acids at the n-terminus of the fragment.

A “variant” is a peptide that is altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition, and substitution with natural and modified amino acids is envisaged. The variant may have conservative amino acid changes, wherein the amino acid being introduced is similar structurally, chemically, or functionally to that being substituted. Generally, the variant will have at least 70% amino acid sequence homology, preferably at least 80% sequence homology, more preferably at least 90% sequence homology, and ideally at least 95%, 96%, 97%, 98% or 99% sequence homology with the reference growth promoting fragment. The variant may include a fragment of the peptide wherein one or more amino acids are deleted. The variant may have 1 to 3 amino acid changes selected from insertion, addition, deletion, and substitution. The variant may have ACE-1 inhibitory activity and/or renin inhibitory activity. Methods to determine these activities are described herein.

“Man-made” as applied to comestible products should be understood to mean made by a human being and not existing in nature.

The term “marine peptide product” is one or more peptides derived from a marine by-product.

The term “marine by product” is a product or discard generated as a result of fish processing, e.g., such filleting. The by products include but are not limited to bloodwaters, fish skin, fish bone and gut waste.

The term “pelagic fish” refers to fish derived from the pelagic zone of the ocean or lake water. Examples include but are not limited to white fish, blue whiting and mackerel.

The term “whitefish” refers to various freshwater salmonid food fish, e.g. genera Coregonus and Prosopium. They have an adipose dorsal fin and inhabit cold lakes and streams.

The term “retentate” is the by-product, or processed by-product, retained by the membrane.

The term “bloodwater” refers to the blood from fish released during fish processing.

The term “fish processing” refers to the processes associated with fish and fish products between the time fish are caught or harvested, and the time that the final product is delivered to the customer. It can extend to any aquatic organism, preferably fish in this context.

The “animal” when used herein may be any animal. Preferably, the animal is a companion animal. Still preferred the animal is a dog or a cat. It may be a rabbit, a hamster, a horse, a guinea pig, a rat, a mouse or a reptile. The animal may be any age. Typically, the animal is an elderly animal. An “elderly animal” is one 8 years and older, typically from 8 to 13 years old.

The term “companion animal” when used herein is an animal kept primarily for a person's company or entertainment rather than as a working animal, livestock or a laboratory animal.

### BRIEF DESCRIPTION OF THE FIGURES

The invention will now be described with reference to the following figures in which;

FIG. 1: illustrates an embodiment of the invention for producing a peptide product from mackerel bloodwaters.

FIG. 2: illustrates oil holding capacity of Blue whiting and Mackerel blood water fractions.

FIGS. 3 A and B: Emulsifying activity (EA) (A) and emulsifying stability (ES) (B) of Blue whiting and Mackerel blood water fractions

FIGS. 4 A and B: Foaming capacity (FC) (A) and foaming stability (FS) (B) of Blue whiting and Mackerel blood water fractions

FIG. 5: Solubility of Blue whiting and Mackerel blood water fractions.

FIG. 6: ACE-I inhibition of Blue whiting and Mackerel blood water fractions at 1 mg/ml

FIG. 7: Renin inhibition of Blue whiting and Mackerel blood water fractions at 1 mg/ml

FIG. 8: SDS-PAGE gel with separated protein bands of samples and unstained protein ladder (UPL)

FIGS. 9 A and B: Water holding capacity (WHC) and oil holding capacity (OHC) of the fish and control gelatine samples

FIG. 10 A to C: Solubility (S), foaming capacity (FC) and foam stability (FS) of the gelatine sample solutions (1%; w/v) at different pH values

FIGS. 11 A and B: Emulsifying activity (EA) and emulsifying stability (ES) of gelatine samples

FIG. 12: Degree of hydrolysis (DH) of SOP1 and SOP2 gelatines with alcalase and papain

FIG. 13: ACE-I inhibition activity of SOP1 and SOP2 gelatine hydrolysates and 53 KDa peptide fractions

FIG. 14: illustrates percentage protein content of recovered bloodwaters for blue whiting and mackerel.

FIG. 15: illustrates the process for making dog biscuits.

FIG. 16: Illustrates essential amino acid content in blood water fraction (horse mackerel)

FIG. 17: Illustrates conditionally essential amino acid content in blood water fraction (horse mackerel)

FIG. 18: Illustrates dispensable amino acid content in blood water fraction (horse mackerel)

FIG. 19: Illustrates essential amino acid content in blood water fraction (mackerel)

FIG. 20: Illustrates conditionally essential amino acid content in blood water fraction (mackerel)

FIG. 21: illustrates dispensable amino acid content in blood water fraction (mackerel)

FIG. 22: Systolic and Diastolic blood pressure for dogs fed the test headed and gutted (H&G) blue whiting hydrolysate containing versus a control.

### DETAILED DESCRIPTION OF THE INVENTION

The current invention relates to a marine peptide product, obtained from the by-products of fish processing. The current inventors have discovered that marine peptide products obtained from fish processing by-products contain peptides which impart health benefits, allowing them to produce a functional food product for animals, as an alternative to “traditional” drugs for prevention or treatment of disease. Notably, the marine peptide product comprises valuable amino acid rich proteins that contain all the essential amino acids. Most notably, the marine peptide product comprises peptides with ACE-1 inhibitory activity.

The food product of the invention not only has health benefits, but it also has a desirable appearance, taste, strength, and texture. Undesirable colour often renders the product undesirable to the dog owner. Notably, the food product of the invention may be a low-calorie food product. The food product is high in total protein content, high in essential amino acid content, high in heart health benefits. In a preferred embodiment, the food product is a dog food product, typically a biscuit.

In an embodiment, the food product may be a biscuit or similar, and may comprise a substitution of a portion of the wheat flour normally used to make a biscuit of this nature, with the protein product of the invention. This can be carried out to accomplish a variation in the protein content and calorie content and the health benefits of the biscuit to the animal. In other words, the amount of the marine peptide product in the biscuit, or indeed any food product of the invention, can be varied depending on the desired type or composition of the biscuit or the intended use of the biscuit. In the prior art, substitution of wheat flour with another ingredient can cause difficulties in the manufacturing of dog biscuits so that texture and appearance of the final product is affected. However, this is not the case when using the marine peptide product of the invention.

The marine peptide product of the invention comprises peptides that can be derived from by-products of fish processing. In this regard, the invention provides a method for producing a marine peptide product from by-products of fish processing. The marine peptide product is the retentate or fraction obtained in the method. The fraction is the permeate.

In one embodiment of the invention, the by-products are captured before they reach the (Dissolved Air Flotation (DAF) systems) system in a fish processing plant and to utilise recovered solids as a source of proteins and amino acids. In one embodiment of the invention, bloodwaters are captured from the tanker, hopper and process lines. Solids are recovered and stored at −4° C. in containers and then passed through a membrane filtration system incorporating 3 kDa and/or 10 kDa fractions that can recover proteins and peptides and fatty acid methyl esters (retentate fraction) and 3 kDa and 10 kDa fractions containing both amino acids and bioactive peptides with potential to impact positively on heart health (through Angiotensin-1-converting enzyme I inhibition—as these fractions contain ACE-I inhibitory peptides).

In an embodiment, the method comprises filtering the bloodwaters from fish processing, e.g., pelagic or white fish processing, with a 3 kDa and/or a 10 kDa membrane, to obtain at least one retentate, a 3 kDa fraction and/or a 10 kDa fraction, each with a particular peptide and amino acid composition. For instance, if a 3 kDa membrane is used, there is a 3 kDa fraction which contains the peptides and a retentate. If a 10 kDa membrane is used, there is a 10 kDa fraction which contains the peptides and there is also a retentate. If both a 3 kDa and 10 kDa membrane is used there will be a retentate for each membrane used.

The retentate and fractions may then be dried. The method of drying may be any suitable method and such methods are known to the skilled person.

In one embodiment, the bloodwaters are filtered through a 3 kDa membrane and a 10 kDa membrane, to obtain a retentate, a 3 kDa fraction and a 10 kDa fraction. The by-products may pass through the membranes at the same time, or it may be sequentially.

In one embodiment, the bloodwaters are filtered through a 3 kDa membrane only.

In one embodiment, the bloodwaters are filtered through a 10 kDa membrane only.

In an embodiment, the pelagic fish is selected from the group comprising mackerel, Blue-whiting, and Horse mackerel. It may be one or more pelagic fish. Preferably, the fish is blue whiting or mackerel.

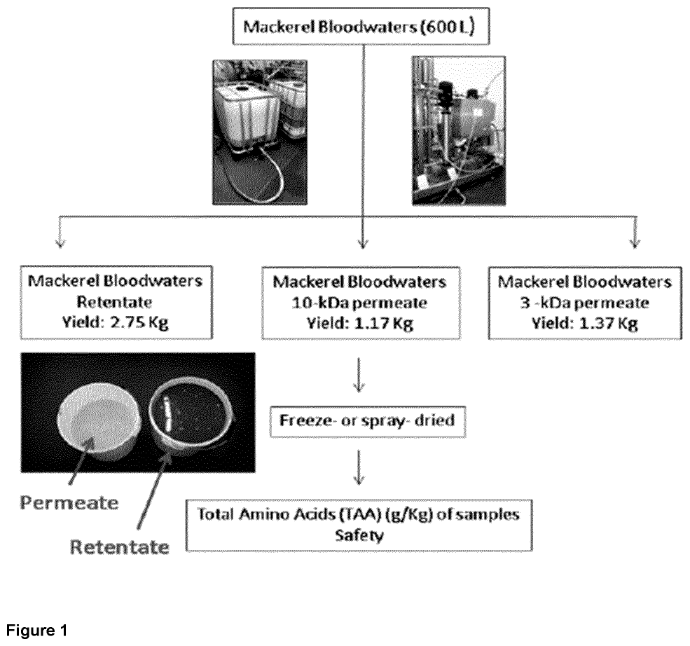

This exemplary method is illustrated in FIG. 1. FIG. 1 illustrates an embodiment of the invention for producing a marine peptide product from mackerel bloodwaters.

In a notable embodiment of this invention, the by-product may be one or more of fish skin, bone and gut waste. The by-product may be obtained from processing of any type of fish, e.g., filleting. Pelagic and white fish are two preferred examples. However, it may be any fish from which collagen/gelatine may be obtained. When the by-product used is one or more of fish skin, bone and gut waste, the method comprises an optional first step of extracting gelatine from the by-product prior to the filtering step.

Any method of extracting gelatine may be used, and such are well known in the art. In an embodiment, of the invention, the gelatine step may include a first “pre-treatment” step as disclosed herein. The extracted gelatine is hydrolysed prior to filtering.

In an embodiment, gelatine does not require extraction. It is possible to hydrolyse the by-product without first extracting the gelatine to provide a hydrolysate. In this embodiment, the hydrolysate is then filtered as per the steps of the method of the invention. The enzymes used are those as described herein.

In a method of the invention, the hydrolysed gelatine, or hydrolysate, may be filtered through a 3 kDa membrane and a 10 kDa membrane to obtain a retentate, a 3 kDa fraction and a 10 kDa fraction. The hydrolysed gelatine, or hydrolysate, may pass through the membranes at the same time, or it may be sequentially.

In one embodiment, the hydrolysed gelatine, or hydrolysate, is filtered through a 3 kDa membrane only.

In one embodiment, the hydrolysed gelatine, or hydrolysate, is filtered through a 10 kDa membrane only.

The flow rate is typically between 20 L per hour and 50 L per hour, typically around 37.5 L per hour.

The method of any embodiment of the invention, after the filtering step, may further comprise a step of freezing, preferably blast freezing, the permeates and retentates. This step is preferred when the product is dried.

In an embodiment, the marine peptide product comprises one or more peptides comprising (or consisting of) a sequence selected from SEQUENCE ID NO. 1 to SEQUENCE ID NO. 188, or a variant thereof.

Preferably, the one or more peptides has ACE-1 inhibitory activity. Typically, the one or more peptides has renin inhibitory activity.

In an embodiment, the marine peptide product comprises one or more peptides comprising (or consisting of) a sequence selected from SEQUENCE ID NO. 1 to SEQUENCE ID NO. 64, or a variant thereof. In an embodiment the product may comprise (or consist of) all of the peptides comprising (or consisting of) a sequence selected from SEQUENCE ID NO. 1 to SEQUENCE ID NO. 64, or variant thereof. In an embodiment, the peptide product may comprise one or more of, or all of, the peptides comprising (or consisting of) SEQUENCE ID NO 1 to 15. These peptides may be obtained from marine by-products, bloodwaters from blue whiting processing. The peptides have a molecular weight less than or equal to 10 KDa.

Preferably, the one or more peptides is selected from SEQUENCE ID NO. 1, 2, 10, 13, 16, 22, 25, 32, 44, 57, 58, 64 and 62 or a variant thereof. All of the peptides may be present.

In an embodiment, the marine peptide product comprises one or more peptides comprising (or consisting of) a sequence selected from SEQUENCE ID NO. 65 to SEQUENCE ID NO. 169, or a variant thereof. In an embodiment the product may comprise (or consist of) all of the peptides comprising (or consisting of) a sequence selected from SEQUENCE ID NO. 65 to SEQUENCE ID NO. 169 or a variant thereof. In an embodiment, the peptide produce may comprise one or more of, or all of, the peptides comprising (or consisting of) SEQUENCE ID NO. 65 to 74. These peptides may be obtained from marine by-products, typically bloodwaters from mackerel processing. The peptides have a molecular weight less than or equal to 10 KDa.

Preferably, the one or more peptides is selected from SEQUENCE ID NO. 122, 123, 133, 139, 141, 147, 148, 150, 152, 157 and 160 or a variant there of. All of the peptides may be present.

In an embodiment, the marine peptide product comprises one or more peptides comprising (or consisting of) a sequence selected from SEQUENCE ID NO. 170 to SEQUENCE ID NO. 188, or a variant thereof. In an embodiment the product may comprise (or consist of) all of the peptides comprising (or consisting of) a sequence selected from SEQUENCE ID NO. 170 to SEQUENCE ID NO. 188 of a variant thereof. In an embodiment, the peptide product may comprise one or more of, or all of, the peptides comprising (or consisting of) SEQUENCE ID No. 170 and 171. These peptides may be obtained from marine by-products, typically blood water from mackerel processing. The peptides have a molecular weight greater than 10 KDa.

Preferably, the one or more peptides is selected from SEQUENCE ID NO. 170, 171, 178, 182, 183, 187, 185, 184 and 186 or a variant thereof. All of the peptides may be present.

In an embodiment, the marine peptide product comprises one or more peptides comprising (or consisting of) a sequence selected from SEQUENCE ID NO. 189 to SEQUENCE ID NO. 271, or 363 or a variant thereof. In an embodiment the product may comprise all of the peptides comprising (or consisting of) a sequence selected from SEQUENCE ID NO. 189 to SEQUENCE ID NO. 271 or 363 or a variant thereof. The peptide product is a hydrolysed product. The peptides are those obtained from marine by-products, typically skin and bones from blue whiting surimi processing. The peptides have a molecular weight less than or equal to 3 KDa.

In an embodiment, the marine peptide product comprises one or more peptides comprising (or consisting of) a sequence selected from SEQUENCE ID NO. 272 to SEQUENCE ID NO. 362, or a variant thereof. In an embodiment the product may comprise all of the peptides comprising (or consisting of) a sequence selected from SEQUENCE ID NO. 272 to SEQUENCE ID NO. 362. The peptide product is a hydrolysed product. The peptides are those obtained from marine by-products, typically skin and bone from blue whiting surimi processing. The peptides have a molecular weight less than or equal to 3 KDa.

Typically, said peptide comprises a maximum 50 amino acids in length. Preferably from about 10 to about 20 amino acids in length.

The marine peptide product may comprise at least 1, preferably at least 2, preferably at least 3, preferably at least 4, preferably at least 5, preferably at least 6, preferably at least 7, preferably at least 8, preferably at least 9, or preferably at least 10 peptides, or preferably at least 20, 25, 30, 35, 45 peptides selected from a peptide comprising (or consisting of) SEQUENCE ID NO. 1 to SEQUENCE ID NO. 188, or a variant thereof.

In one embodiment, the marine peptide product comprises at least one or more of a peptide comprising (or consisting of) SEQUENCE ID NO. 1, SEQUENCE ID NO 2, SEQUENCE ID NO 3, SEQUENCE ID NO 4, SEQUENCE ID NO 5, SEQUENCE ID NO 6, SEQUENCE ID NO 7, SEQUENCE ID NO 8, SEQUENCE ID NO 9, SEQUENCE ID NO 10, SEQUENCE ID NO 11, SEQUENCE ID NO 12, SEQUENCE ID NO 13, SEQUENCE ID NO 14 and SEQUENCE ID NO 15. In one embodiment, the marine peptide product comprises at least all of the peptides comprising (or consisting of) SEQUENCE ID NO. 1, SEQUENCE ID NO 2, SEQUENCE ID NO 3, SEQUENCE ID NO 4, SEQUENCE ID NO 5, SEQUENCE ID NO 6, SEQUENCE ID NO 7, SEQUENCE ID NO 8, SEQUENCE ID NO 9, SEQUENCE ID NO 10, SEQUENCE ID NO 11, SEQUENCE ID NO 12, SEQUENCE ID NO 13, SEQUENCE ID NO 14, and SEQUENCE ID NO 15. The product may comprise one or more additional peptides of the invention.

In one embodiment, the marine peptide product comprises at least one or more of a peptide comprising (or consisting of) SEQUENCE ID NO. 65, SEQUENCE ID NO 66, SEQUENCE ID NO 67 SEQUENCE ID NO 68, SEQUENCE ID NO 69, SEQUENCE ID NO 70, SEQUENCE ID NO 71, SEQUENCE ID NO 72, SEQUENCE ID NO 73, and SEQUENCE ID NO 74. In one embodiment, the marine peptide product comprises at least all of the peptides comprising (or consisting of) SEQUENCE ID NO. 65, SEQUENCE ID NO 66, SEQUENCE ID NO 67 SEQUENCE ID NO 68, SEQUENCE ID NO 69, SEQUENCE ID NO 70, SEQUENCE ID NO 71, SEQUENCE ID NO 72, SEQUENCE ID NO 73, and SEQUENCE ID NO 74. The product may comprise one or more additional peptides of the invention.

In one embodiment, the marine peptide product comprises at least one or more of a peptide comprising (or consisting of) SEQUENCE ID NO. 170 and SEQUENCE ID NO 171. In one embodiment, the marine peptide product comprises at least the peptides comprising (or consisting of) SEQUENCE ID NO. 170 and SEQUENCE ID NO 171. The product may comprise one or more additional peptides of the invention.

In one embodiment, the peptide of the invention is modified. In one embodiment the peptide is modified with a protecting group. In one embodiment, the peptide is modified to increase its lipophilicity. In one embodiment, the peptide is modified to increase its half-life. In one embodiment, an N or C-terminal amino acid of the peptide is modified. In one embodiment, the N or C-terminal amino acid of the peptide is modified with a protecting group.

The invention provides a peptide comprising (or consisting of) a sequence selected from SEQUENCE ID NO. 1 to SEQUENCE ID NO. 363, or a variant thereof.

The invention provides a peptide comprising (or consisting of) a sequence selected from SEQUENCE ID NO. 1 to SEQUENCE ID NO. 188, or a variant thereof.

Preferably, the peptide is selected from SEQUENCE ID NO. 1, 2, 10, 13, 16, 22, 25, 32, 44, 57, 58, 64 and 62 or a variant thereof.

Still preferred, the peptide is selected from SEQUENCE ID NO. 122, 123, 133, 139, 141, 147, 148, 150, 152, 157 and 160 or a variant thereof.

In an embodiment, the peptide is selected from SEQUENCE ID NO. 170, 171, 178, 182, 183, 187, 185, 184 and 186 or a variant thereof.

The invention provides a marine peptide product obtained by the method of the invention. The peptide product may be one comprising peptides of 3 kDa or less. The peptide product may be one comprising peptides of 10 KDa or less, or from 3 kDa to 10 kDa. The peptide product may be one comprising peptides greater than 10 KDa. The marine peptide product may be one obtained from skin and/or bone derived from processing of blue whiting into surimi products. Alternatively, the marine peptide product may be one obtained from bloodwater from pelagic or whitefish processing.

In an embodiment, the invention provides a marine peptide product comprising a 10 kDa fraction obtained from bloodwater. The bloodwater may be from pelagic fish processing, such as mackerel and blue whiting.

In an embodiment, the invention provides a marine peptide product comprising a 3 kDa fraction obtained from by-products of fish processing, in which the by product is gelatine extracted from fish by products selected from the group comprising fish skin, fish bones and gut waste. The fish may be any collagen containing fish, such as blue whiting. The fish processing may be blue whiting processing into surimi products.

Preferably, the marine peptide product may further comprise at least one fatty acid. The fatty acids may be one or more of the fatty acids listed in Table 5. The fatty acid may be all of the fatty acids listed in Table 5.

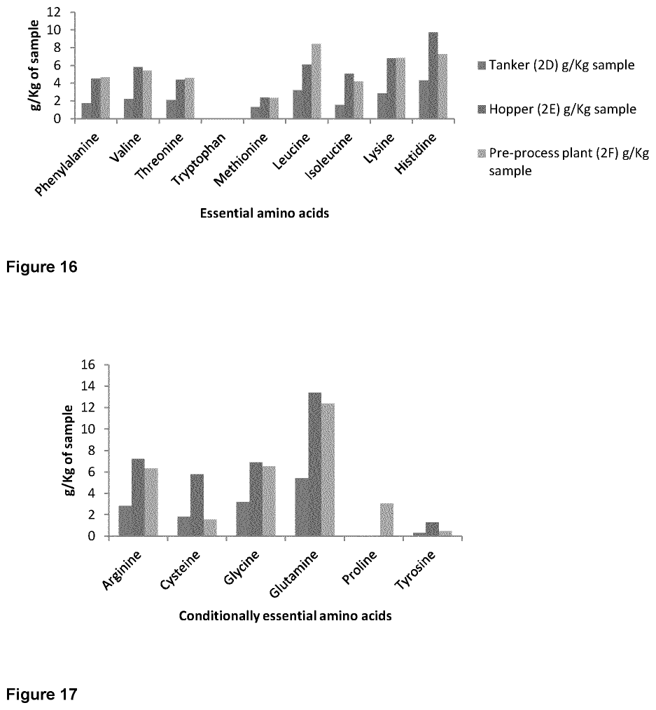

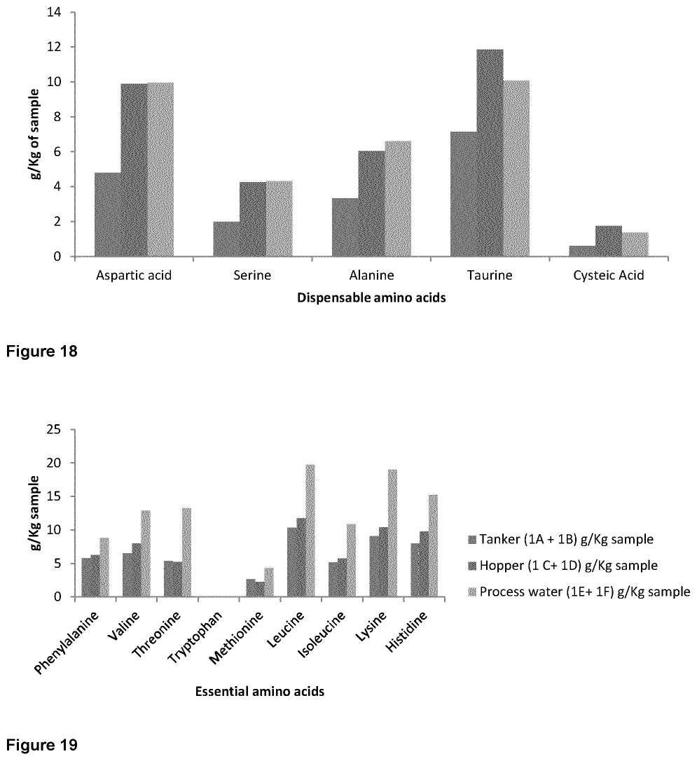

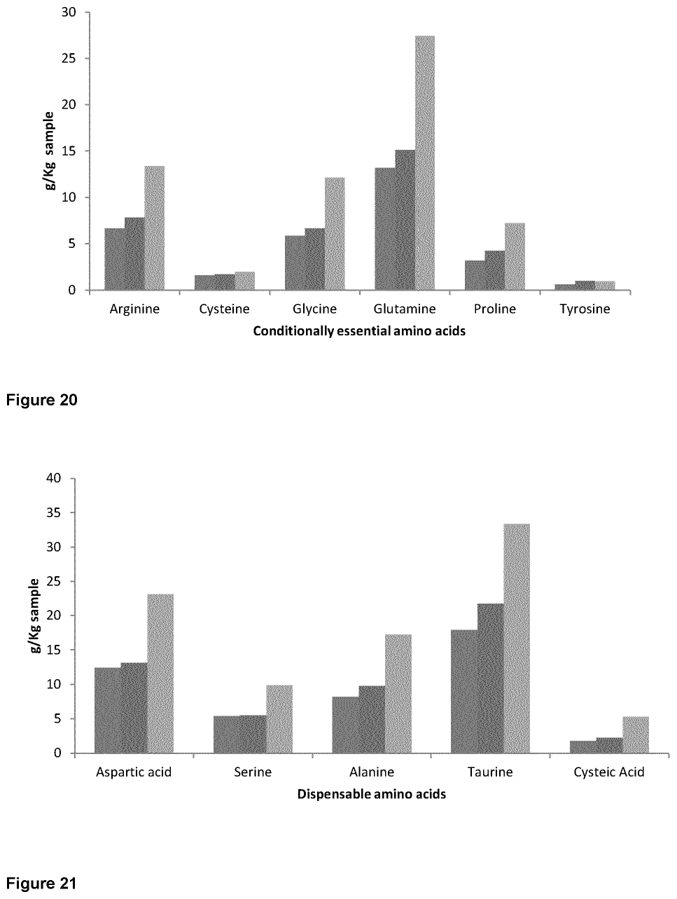

The peptide product of the invention has a particular peptide and amino acid composition. For example, the essential, non-essential and conditionally essential quantities for bloodwaters are illustrated by FIGS. 16 to 21.

In one embodiment, the total amino acid content of mackerel bloodwater hkDa permeate extract (Mackerel) was determined as 199.94 g/kg DW and the content of bloodwater 10 kDa (blue whiting) was determined as 35.31 g/kg DW with the essential amino acid composition approximately 30.41% of the total amino acid composition for Mackerel 10 kDa and 24.62% for Blue whiting 3 kDa, respectively.

The level of the essential amino acids lysine and methionine contained in Mackerel 10-kDa blood-water extracts were comparable to flaxseed protein sources (Ufaz & Galili, 2008) but less than whey protein isolate. The blue-whiting 10-kDa permeate fraction was rich in taurine (14.977 g/Kg D) as was the Mackerel 10-kDa permeate (63.411 g/kg D and taurine levels in all retentates and permeate fractions were greater than either whey protein isolate or flaxseed protein isolate with the exception of the 3 kDa fraction for Mackerel.

These results are outlined in Table below, which shows the content of amino acids in bloodwater retentate fraction, 10 kDa fraction (mackerel and blue whiting), 3 kDa fraction, whey protein isolate and flax.

| | Mackerel | Mackerel | Mackerel | Blue whiting | Whey Protein | |
|---|---|---|---|---|---|---|
| Amino acid | retentate | 10-kDa | 3-kDa | 10-kDa | Isolate | Flax |
| Cysteic acid | 3.672 | 2.731 | 1.536 | 1.232 | 43.539 | 7.215 |
| Taurine | 25.108 | 63.411 | 2.843 | 14.977 | 16.873 | 11.896 |
| Methionine | 3.739 | 4.234 | 43.98 | 0.592 | 32.327 | 7.969 |
| Asparagine | 15.257 | 8.381 | 0.593 | 0.375 | 112.512 | 28.806 |
| Threonine | 6.941 | 6.395 | 0.646 | 0.365 | 46.246 | 15.065 |
| Serine | 5.344 | 6.264 | 0.257 | 0.193 | 34.299 | n.d. |
| Glutamic acid | 13.678 | 12.301 | 1.204 | 1.05 | 156.697 | 56.043 |
| Glycine | 5.311 | 15.369 | 0.543 | 2.306 | 14.201 | 16.611 |
| Alanine | 8.133 | 16.83 | 0.421 | 2.916 | 49.764 | 13.11 |
| Cysteine | 3.854 | 4.221 | 4.492 | 2.608 | 4.266 | 2.497 |
| Valine | 7.541 | 6.517 | 0.401 | 0.728 | 46.654 | 14.206 |
| Isoleucine | 5.108 | 3.213 | 0.293 | 0.685 | 51.073 | 11.41 |
| Leucine | 9.777 | 6.479 | 0.4 | 0.888 | 129.471 | 18.444 |
| Phenylalanine | 5.574 | 2.7 | 0.734 | 0.211 | 30.608 | 13.554 |
| Histidine | 8.318 | 19.122 | 3.809 | 4.774 | 18.641 | 7.335 |
| Lysine | 8.573 | 12.182 | 0.907 | 0.458 | 102.09 | 11.089 |
| Arginine | 5.388 | 9.596 | 1.42 | 0.144 | 21.752 | 28.173 |
| Proline | 3.368 | n.d. | 0.82 | n.d. | 37.679 | 8.382 |
| Tyrosine | n.d. | n.d. | 0.59 | 0.81 | 27.467 | 7.963 |
| Total g/Kg | 144.684 | 199.946 | 65.889 | 35.312 | 976.159 | 279.768 |

Table 1: Amino Acids

The levels of taurine in fish bloodwater fractions analysed in BBPP were between 0.2 to 2 g/100 g, which is considerably high compared to beef and pork muscle with taurine content of 50-100 mg/100 g and 118 mg/100 g, respectively. A variety of studies have identified the beneficial effects of taurine in the treatment of cardiovascular diseases, renal dysfunction, and retinal neuron damage and these bloodwaters could be useful in the development of complementary pet foods such as treats to enhance taurine levels for improved heart heath and renal function.”.

A still further aspect provides a food product, or comestible product, comprising the marine peptide product obtained by the method of the invention. The food product is preferably a biscuit for consumption by a dog or cat. The biscuit may be any colour or shape.

An aspect of the invention provides a marine peptide product or food product of the invention, for use in a method of treatment or prevention of disease in an animal, typically prevention. As shown in the accompanying examples, the marine peptide product of the invention has ACE-1 inhibition activity. The marine peptide product also has Renin inhibition activity. Therefore, it has an application for prevention or treatment of heart disease or renal disease in animals, particularly cats and dogs. The food product of the invention is intended as a functional food product and could be used as an alternative to traditional drugs or medicine.

Notably, the current invention provides the use to treat or prevent hypertension in elderly animal, preferably dogs. Typically, this may be hypertension that has resulted from obesity, inflammation and/or proteinuria in the animal. In dogs with marked proteinuria ACE-1 inhibitors may also have reno-protective effects and reduce the magnitude of proteinuria.

A method of treatment or prevention of a disease in an animal is also provided by the current invention comprising administration of the peptide product or peptide of the current invention to an animal. The disease may be selected from the group comprising heart disease and renal disease or an inflammatory disease or condition. The condition may be high blood pressure or hypertension or proteinuria. The heart disease may be selected from the group comprising, but not limited to, chronic heart failure (CHF), chronic valvular disease, hypertrophic cardiomyopathy, mitral valve regurgitation (MR), e.g., secondary to degeneration of the mitral valve apparatus, one associated with a metabolic syndrome, for examples, heart health or congestive heart failure and chronic renal failure (CRF).

It will be appreciated that other ingredients or additives may be added to the food product of the invention. For example, an aroma enhancing ingredient, e.g. Fats (fish derived or tallow) can be added for aroma enhancement.

Unlike conventional biscuit formulas, no salt is added in the most preferred embodiments of this invention. This embodiment of the invention provides higher quality protein or essential amino acids compared to traditional wheat flour biscuits. The total protein content of the biscuit embodiments can be selectively adjusted to provide eye health (cats) or heart health (dogs) benefits.

Also provided is a method for producing a marine peptide product from a mackerel by-product derived from fish processing, the method comprising: hydrolysing the mackerel by-product, which is one or more of fish skins, fish bone and gut waste, with one or more hydrolyses enzymes selected from alcalase and papain, using one or more filters selected from 3 kDa filter membrane and 10 kDa filter membrane, to obtain at least one retentate, a 3 kDa fraction and/or a 10 kDa fraction, wherein the marine peptide product is the obtained fraction(s).

The invention will now be described with reference to the following examples.

### EXAMPLES

### Example 1: Bloodwaters from Pelagic Fish Processing

Sampling and Stabilisation of Pelagic Blood-Waters

One litre (1 L)×3 samples of either Refrigerated seawater (RSW) from the supply tanker, hopper or process-line containing fish blood from Mackerel, Blue-whiting or Horse mackerel were collected independently from five different processors (Table 2) during the period September-April 2016-2017. Samples were collected in 1 L bottles and transported immediately to the laboratory on ice where they were frozen at −80° C. and subsequently freeze-dried using an industrial scale FD 80 model freeze-drier (Cuddon Engineering, New Zealand), milled and stored at −20° C. until further use. The dry matter content of the samples was calculated following drying. The pH of the samples was determined at 20° C. using pH standard pH meter and a calibrated Hamilton double pore electrode.

Up-Scaled Fractionation of Bloodwaters Using Molecular Weight Cut Off Filtration (MWCO) Skid System

The concentration SKID (FDT engineering, Ireland) shown in FIG. 1 and fitted with 3 kDa and 10 kDa UF Koch membranes (Millipore, Cork, Ireland) was used for pilot scale concentration of blood-waters collected from the hopper during processing of Mackerel and Blue-whiting independently. Blue-whiting and mackerel blood waters were collected in 600 L containers in Killybegs in April 2018, frozen for 24 hrs and transported to the Teagasc Food Research Centre Ashtown where they were passed through the concentration SKID to produce retentate and permeate products (FIG. 1). The flow rate was 37.5 L per hr and 600 L were processed in a 4 hr time period. The 3 and 10 kDa permeates and retentates produced were blast frozen for 30 mins and subsequently freeze-dried as previously described. Dry weight yields were calculated, and products stored at −80° C. until further analysis.

Total Protein and Polypeptide Profile

The total protein content of all samples was analysed using the Bicinchoninic Acid Assay (BCA assay) using bovine serum albumin as a standard. In addition, proximate analysis was used to quantify protein, ash and lipid content of the samples. The total protein content was determined in triplicate using a LECO FP628 Protein analyser (LECO Corp., MI, USA) based on the Dumas method and according to AOAC method 992.15, 1990. The conversion factor of 6.25 was used to convert total nitrogen to protein. The total protein content and yields were calculated per litre of blood and are shown in FIG. 14. Moisture and ash content were determined gravimetrically in accordance with previously described methods (Kolar 1992).

Physical and Chemical Properties Determination of Blood Water Fractions

Proximate Analysis

The yield of stabilized products was calculated after freeze-drying and weighing of the samples and is expressed as a percentage of the product in total mass of initial blood water. The total protein content of the samples was determined using the Dumas combustion method using a LECO FP328 Protein analyser (LECO Corp, MI USA), according to Association of Official Analytical Chemists (AOAC) method 992.15 (AOAC, 1990). The conversion factor of 6.25 was used to convert total nitrogen to protein.

The ash content was determined gravimetrically, as previously described (Kolar, 1992).

The total fat content was determined gravimetrically using Ankom XT15 Extractor (Ankom Technology, Macedon NY, USA) for lipid extraction, after previous acid hydrolysis using Ankom HCl Hydrolysis System according to manufacturers' operating manual.

Water Activity

Water activity (aw) of all samples was measured using an AquaLab Lite meter (Decagon Devices Inc., Germany). Approximately 0.25 g of finely powdered sample was placed in the water activity metre and aw and the temperature was recorded.

Total and Amino Acid (TAA) Analysis of Samples

For total amino acid composition analysis, recovered blood-water samples were hydrolyzed in 6M HCl at 110° C. for 23 hours following the method of Hill (1965). Samples were then de-proteinised by mixing equal volumes of 24% (w/v) tri-chloroacetic acid (TCA) and sample, these were allowed to stand for 10 minutes before centrifuging at 14400×g (Microcentaur, MSE, UK) for 10 minutes. Supernatants were removed and diluted with 0.2 M sodium citrate buffer, pH 2.2 to give approximately 250 nmol of each amino acid residue. Samples were then diluted 1 in 2 with the internal standard norleucine, to give a final concentration of 125 nm/mL. Amino acids were quantified using a Jeol JLC-500N amino acid analyser (Jeol (UK) Ltd., Garden city, Herts, UK) fitted with a Jeol Na+ 133 high performance cation exchange column.

Fatty Acid Methyl Ester (FAME) Analysis

Lipids from samples of Blue whiting and Mackerel 3 KDa and 10 KDa permeates and retentates were extracted using ethanol and ethyl acetate mixture (1:1, w/w), as described by Lin et al. (2004). Extracted lipids were then directly converted to FAMEs using boron-trichloride in methanol (14%, w/v) without previous derivatization, with slight modifications of the method previously described by Araujo et al. (2008). Separation and analysis of the FAMEs was done using Agilent 7890A/5975C GC-MSD system (Agilent Technologies, Santa Clara, CA, USA) equipped with Agilent J&W DB-FastFAME column (30 m×0.25 mm, 0.25 μm). Slightly modified method from Agilent Application note 5991-8706EN was used for analysis (Zou and Wu, 2018). Hydrogen was used as carrier gas in constant pressure mode at 8 PSI and sample injection volume was 1 μL in inlet split mode (25:1). Temperature program of oven was as follows: 50° C. (0.5 minutes), then 15° C./min to 194° C. (4 minutes) and finally 4° C./min to 240° C. (1 minute). Mass spectra were acquired in scan mode in the 40-550 AMU mass range.

Chromatographic peaks (total ion chromatogram, TIC) were identified by comparison of retention times (RT) and mass spectra with peaks of analysed FAMEs in the standard mixture. Identification was confirmed by searching the generated MS spectra within available spectral database (NIST11) using Agilent ChemStation software (v X.XX). Supelco Fame 37 mix (Sigma Aldrich, Darmstadt, Germany) was utilized for external calibration by making series of appropriate dilutions with hexane, with individual compound peaks used to construct a five-point calibration curve. Each individual FAME is quantified by measuring response (peak area after integration) of a selected quantifier ion from the compound spectrum using ChemStation software. 1 ml of glyceryl-tri-heptadecanoate (Sigma Aldrich, Darmstadt, Germany) in hexane (1 mg/ml, w/v) was added to lipid samples prior to derivatization, to correct for transesterification efficiency and procedural losses. All samples were analysed in triplicates and results expressed as concentration (mg/g) of each fatty acid in extracted fat.

Determination of Techno-Functional Properties

Water and Oil Holding Capacities

The water holding capacity (WHC) and oil holding capacity (OHC) were measured using the modified methods described by Garcia-Vaquero et al. (2017). In brief, 100 mg of each fraction sample was mixed with 1 ml of distilled water or oil (olive and sunflower oil) using a vortex mixer (VV3, VWR International Ltd., Ireland) for 30 seconds. The suspension was then centrifuged at 2200×g for 30 min (Eppendorf MiniSpin, Eppendorf UK Limited, United Kingdom) at 19° C. The supernatant was then decanted by draining the tubes at 45° angle for 10 min (20 min for oil). Water/oil holding capacity was calculated by dividing the weight of water/oil absorbed by the weight of the protein sample and expressed as grams of water or sunflower oil held by 1 g of protein.

Solubility

Solubility in water (S) was determined using the modified method by Ogunwolu et al. (2009). 100 mg of each sample was dispersed in 10 ml deionised water. Using pH-meter, the pH of the dispersions was adjusted to 2, 4, 6, 8 and 10 with 0.1 N HCl and 0.1 N NaOH. The mixture was vortexed at room temperature (19-20° C.) for 5 min, and subsequently centrifuged at 7500×g for 15 min (Lynx 6000, Thermo Fisher Scientific, MA USA). The protein content in the supernatant was determined using the Pierce™ BCA Protein Assay Kit (Thermo Fischer Scientific, MA, USA). Solubility of samples at different pH conditions was then calculated as:

S (%)=(protein content in the supernatant/protein content in full dispersion)×100.

Foaming Capacity

The foaming capacity (FC) of the blood water permeates and retentate samples were determined by the modified method described by Garcia-Vaquero et al. (2017). Samples were suspended in deionized water to make 1% concentration (w/v) and the pH was adjusted to 2, 4, 6, 8 and 10 with 0.1 N HCl and 0.1 N NaOH. The suspensions were homogenized using a T 25 digital ULTRA-TURRAX® homogenizer (IKA®, Germany) (10,000 rpm; 1 min) and the volume of produced foam was measured in a graduated 50 ml Falcon tube. FC was calculated as the volume of foam using the formula:

FC (%)=(Vf−V0)V0×100

where; V0 is the initial volume of protein solution before homogenization and Vf is the volume of foam produced after homogenization. The foaming stability (FS) was calculated using the same formula, with Vf measured after 15, 30 and 60 min.

Emulsifying Activity and Emulsifying Stability

The emulsifying activity (EA) of the blood water permeates and retentate samples were determined using a modified method of Garcia-Vaquero et al. (2017). Each sample was suspended in deionized water to make 1% concentration (w/v) and the pH was adjusted to 2, 4, 6, 8 and 10 with 0.1 N HCl and 0.1 N NaOH. The solution was homogenized for 30 s at 14,000 rpm using a T 25 digital ULTRA-TURRAX® homogenizer (IKA®, Germany). The emulsion was created by addition of sunflower oil to aqueous phase (oil:sample solution=3:2) in 50 ml Falcon tubes. The oil was added in two steps: half of the volume was first added, and the mixture was homogenized 30 s at 14000 rpm, after which the rest of the oil was added and homogenization was repeated for 90 s at the same speed. The tubes containing emulsion were then centrifuged at 1100×g for 5 min and the volume of the emulsion layer was recorded. Emulsifying activity was calculated by the formula:

EA (%)=(Ve/Vt)×100

Being Ve the volume of the emulsion layer after centrifuging and Vt the all volume inside the tube. Emulsion stability (ES) was determined by heating the previously prepared emulsions at 85° C. for 15 min, cooling at room temperature for 10 min and centrifuging again 1100×g for 5 min. The ES was calculated and expressed as the % of EA after heating and centrifuging centrifuging by the formula:

ES (%)=(V_{emulsion after heating}/V_{original emulsion})×100

ACE-I Inhibition Assay

ACE-I (angiotensin-converting enzyme 1) inhibition bioassay of permeate and retentate samples (Mackerel and Blue whiting) was carried out according to the manufacturer's instructions (ACE Kit—WST, Dojindo Laboratories, Kumamoto, Japan). In brief, 20 μL of each sample aqueous solution at a concentration of 1 mg/mL was added to 20 μL substrate and 20 μL enzyme working solution in triplicate. Captopril was used as a positive control. Samples were incubated at 37° C. for 1 h. A 200 μL of indicator working solution was then added to each well, and subsequent incubation at room temperature was carried out for 10 min. Absorbance at 450 nm was read using a FLUOstarOmega microplate reader (BMG LABTECH GmbH, Offenburg, Germany). The percentage of inhibition was calculated using the following equation:

% ACE-I inhibition=100% Initial activity−Inhibitor×100/100% Initial activity

Renin Inhibition Assay

Renin inhibition activity of permeates and retentate fractions was tested in vitro for renin. The bioassay was carried out according to the manufacturer's instructions (Renin Inhibitor Screening Assay Kit, Cayman chemical, Ann Harbor, MI, USA). In brief, 10 μL of each fraction solution in dimethyl-sulfoxide (DMSO) at a concentration of 1 mg/mL was added to 20 μL substrate and 150 μL of assay buffer in triplicate. Renin was then added to 100% initial activity and inhibitor wells, the wells were shaken for 10 seconds and incubated at 37-C for 15 min. Fluorescence (excitation at 345 nm and emission at 490 nm) was read using a FLUOstarOmega microplate reader (BMG LABTECH GmbH, Offenburg, Germany). The percentage of inhibition was calculated using the following equation:

% Renin inhibition=100% Initial activity−Inhibitor×100/100% Initial activity

Peptide Identification by Tandem Mass Spectrometry

The nano-LC-MS/MS analysis was performed using an Eksigent Nano-LC Ultra 1D Plus system (Eksigent of AB Sciex, CA) coupled to the quadrupole-time-of-flight (Q-ToF) TripleTOF® 5600 system from AB Sciex Instruments (Framingham, MA) that is equipped with a nano-electrospray ionization source.

Samples were re suspended in 50 μL of 0.1% TFA in 2% ACN. 2 μl of every sample were loaded onto a trap column (Nano-LC Column, 3μ C18-CL, 350 μm×0.5 mm; Eksigent) and desalted with 0.1% TFA at 3 μl/min during 5 min. After 5 min of preconcentration, the trap column was automatically switched in-line onto a nano-HPLC capillary column (3 μm, 75 μm×12.3 cm, C18) (Nikkyo Technos Co, Ltd. Japan). Mobile phase A contained 0.1% v/v formic acid in water, and solvent B, contained 0.1% v/v FA in 100% acetonitrile. A linear gradient from 5% to 35% of solvent B over 60 min at a flow rate of 0.3 μL/min and running temperature of 30° C. was used for chromatographic separations.

The column outlet was directly coupled to a nano-electrospray ion (nESI) source. Operating conditions for the Q/ToF mass spectrometer were positive polarity and data-dependent mode. Sample was ionized applying 2.8 kV to the spray emitter. Survey MS1 scans were acquired from 350-1250 m/z for 250 ms. The quadrupole resolution was set to ‘UNIT’ for MS2 experiments, which were acquired 100-1500 m/z for 50 ms in ‘high sensitivity’ mode. Following switch criteria were used: charge: 1+ to 5+; minimum intensity; 70 counts per second (cps). Up to 25 ions were selected for fragmentation after each survey scan. Dynamic exclusion was set to 15 s. The system sensitivity was controlled with 2 fmol of 6 standard proteins (LC Packings).

Data Analysis

Automated spectral processing, peak list generation, and database search for the identification of the peptides were performed using Mascot Distiller v2.7.1.0 software (Matrix Science, Inc., Boston, MA) (hppt://www.matrixscience.com) using MascotServer v2.6 (Matrix Science, Inc., Boston, MA) (hppt://www.matrixscience.com).

The UniProt protein database was used to identify the peptides with a significance threshold p<0.05. Used taxonomy for the identification in database was chordata organisms. The tolerance on the mass measurement was 0.3 Da in MS mode and 100 ppm in MS/MS ions.

In Silico Digestion of Identified Peptides

The identified peptides were digested in silico using BioPep online analysis tools (http://www.uwm.edu.pl/biochemia/index.php/en/biopep), with pepsin (EC 3.4.23.1), trypsin (EC 3.4.21.4) and chymotrypsin A (EC 3.4.21.1) in order to simulate gastric digestion process (Minkiewicz et al., 2008). The results of in silico enzyme hydrolysis was then searched for active fragments in BioPep database to identify potential ACE-I inhibitory peptides that may be released during digestion.

Results

Yield and Proximate Composition of Blood Water Fractions

The freeze-dried filtration fractions had a wide range of protein content, with Blue whiting 3 KDa permeate (BW 3 KDa) containing only 11.1 (±1.53) % of protein, up to 40.5 (±0.16) % in Markerel 10 KDa permeate (Mac 10 KDa) sample. Other filtration fractions, Blue whiting 10 KDa permeate (BW10 KDa), Blue whiting retentate (BW Ret), Mackerel 3 KDa permeate (Mac 3 KDa) and Mackerel retentate (Mac Ret) had protein content between those values (13.3±2.17%, 28.9±0.78%, 21.5±0.84% and 29.9±0.45%, respectively). All permeate fractions had a low lipid content (<1%), while BW Ret and Mac Ret had 1.55 (±0.20) % and 1.69 (±0.11) % fat, respectively. All dried fractions had ash content over 50%, with BW 3 KDa and BW 10 KDa containing over 70% (73.0±0.98% and 75.8±2.01%, respectively) and Mac 3 KDa containing 67.4±1.91% ash. Bw Ret and Mac Ret retentate fractions and Mac 10 KDa permeate fraction contained similar ash content (50.2±0.31%, 51.9±0.49% and 53.0±0.18%, respectively). All the tested fractions had water activity (Aw) values below 0.3, which indicates proper drying of the samples and ensures good microbial stability.

TABLE 2
Proximate composition and water activity of Blue
whiting and Mackerel blood water fractions

| | Crude | | | |
|---|---|---|---|---|
| | protein | Lipid | Ash | |
| | (%, w/w) | (%, w/w) | (%, w/w) | Aw |
| BW 3 KDa | 11.1 ± 1.53 | <1 | 73.0 ± 0.98 | 0.15 ± 0.03 |
| BW 10 KDa | 13.3 ± 2.17 | <1 | 75.8 ± 2.01 | 0.28 ± 0.07 |
| BW Ret | 28.9 ± 0.78 | 1.55 ± 0.20 | 50.2 ± 0.31 | 0.27 ± 0.07 |
| Mac 3 KDa | 21.5 ± 0.84 | <1 | 67.4 ± 1.91 | 0.23 ± 0.00 |
| Mac 10 KDa | 40.5 ± 0.16 | <1 | 53.0 ± 0.18 | 0.25 ± 0.00 |
| Mac Ret | 29.9 ± 0.45 | 1.69 ± 0.11 | 51.9 ± 0.49 | 0.19 ± 0.00 |

The total amino acid (TAA) content of Mackerel blood water fractions is presented in Table 3. The Mac Ret and Mac 10 KDa samples had significantly higher total amino acid content (144.7 and 199.9 g/Kg, respectively) than Mac 3 KDa fraction (65.9 g/Kg). Most abundant detected amino acids were taurine (25.1 g/Kg, 63.4 g/Kg and 2.84 g/Kg in Mac Ret, Mac 10 KDa and Mac 3 KDa, respectively), asparagine (15.3 g/Kg, 8.38 g/Kg and 0.59 g/Kg in Mac Ret, Mac 10 KDa and Mac 3 KDa, respectively) and glutamic acid (13.3 g/Kg, 12.3 g/Kg and 1.20 g/Kg in Mac Ret, Mac 10 KDa and Mac 3 KDa, respectively). All essential amino acids were detected in samples, except tyrosine and tryptophan (which is degraded by acid hydrolysis during sample preparation), with significant concentration of lysine (8.57 g/Kg, 12.2 g/Kg and 0.91 g/Kg in Mac Ret, Mac 10 KDa and Mac 3 KDa, respectively) in all samples.

TABLE 3
Total amino acid (TAA) composition of Blue whiting
and mackerel 10 and 3 kDa filtrates respectively

| | | | | Blue |
|---|---|---|---|---|
| | Mac | Mac | Mac | whiting |
| | Ret | 10 KDa | 3 KDa | 10-kDa |
| Amino acid | (g/Kg) | (g/Kg) | (g/Kg) | (g/Kg) |
| Cysteic acid | 3.67 | 2.73 | 1.54 | 1.23 |
| Taurine | 25.1 | 63.4 | 2.84 | 14.9 |
| Methionine | 3.74 | 4.23 | 43.9 | 0.59 |
| Asparagine | 15.3 | 8.38 | 0.59 | 0.38 |
| Threonine | 6.94 | 6.39 | 0.65 | 0.37 |
| Serine | 5.34 | 6.26 | 0.26 | 0.19 |
| Glutamic acid | 13.7 | 12.3 | 1.20 | 1.05 |
| Glycine | 5.31 | 15.4 | 0.54 | 2.31 |
| Alanine | 8.13 | 16.8 | 0.42 | 2.92 |
| Cysteine | 3.85 | 4.22 | 4.49 | 2.61 |
| Valine | 7.54 | 6.52 | 0.40 | 0.73 |
| Isoleucine | 5.11 | 3.21 | 0.29 | 0.69 |
| Leucine | 9.78 | 6.48 | 0.40 | 0.89 |
| Phenylalanine | 5.57 | 2.70 | 0.73 | 0.21 |
| Histidine | 8.32 | 19.1 | 3.81 | 4.78 |
| Lysine | 8.57 | 12.2 | 0.91 | 0.46 |
| Arginine | 5.39 | 9.59 | 1.42 | 0.14 |
| Proline | 3.37 | n.d. | 0.82 | n.d. |
| Tyrosine | n.d. | n.d. | 0.59 | 0.81 |
| Total (g/Kg) | 144.7 | 199.9 | 65.9 | 35.3 |

TABLE 4
Total amino acid (TAA) composition (g/Kg) of pelagic bloodwaters recovered
using MWCO filtration and drying. The mackeral and blue whiting retentates
and the 10 kDa and 3 kDa fractions are showed comparable to whey
protein isolate TAA content and flax TAA content

| | Mackerel | Mackerel | Mackerel | Blue whiting | Whey Protein | |
|---|---|---|---|---|---|---|
| Amino acid | retentate | 10-kDa | 3-kDa | 10-kDa | Isolate | Flax |
| Cysteic acid | 3.672 | 2.731 | 1.536 | 1.232 | 43.539 | 7.215 |
| Taurine | 25.108 | 63.411 | 2.843 | 14.977 | 16.873 | 11.896 |
| Methionine | 3.739 | 4.234 | 43.98 | 0.592 | 32.327 | 7.969 |
| Asparagine | 15.257 | 8.381 | 0.593 | 0.375 | 112.512 | 28.806 |
| Threonine | 6.941 | 6.395 | 0.646 | 0.365 | 46.246 | 15.065 |
| Serine | 5.344 | 6.264 | 0.257 | 0.193 | 34.299 | n.d. |
| Glutamic acid | 13.678 | 12.301 | 1.204 | 1.05 | 156.697 | 56.043 |
| Glycine | 5.311 | 15.369 | 0.543 | 2.306 | 14.201 | 16.611 |
| Alanine | 8.133 | 16.83 | 0.421 | 2.916 | 49.764 | 13.11 |
| Cysteine | 3.854 | 4.221 | 4.492 | 2.608 | 4.266 | 2.497 |
| Valine | 7.541 | 6.517 | 0.401 | 0.728 | 46.654 | 14.206 |
| Isoleucine | 5.108 | 3.213 | 0.293 | 0.685 | 51.073 | 11.41 |
| Leucine | 9.777 | 6.479 | 0.4 | 0.888 | 129.471 | 18.444 |
| Phenylalanine | 5.574 | 2.7 | 0.734 | 0.211 | 30.608 | 13.554 |
| Histidine | 8.318 | 19.122 | 3.809 | 4.774 | 18.641 | 7.335 |
| Lysine | 8.573 | 12.182 | 0.907 | 0.458 | 102.09 | 11.089 |
| Arginine | 5.388 | 9.596 | 1.42 | 0.144 | 21.752 | 28.173 |
| Proline | 3.368 | n.d. | 0.82 | n.d. | 37.679 | 8.382 |
| Tyrosine | n.d. | n.d. | 0.59 | 0.81 | 27.467 | 7.963 |
| Total g/Kg | 144.684 | 199.946 | 65.889 | 35.312 | 976.159 | 279.768 |

The fatty acid (FA) profile of Mackerel and Blue whiting retentates and 10 KDa permeate fractions is presented in Table 5. The content and profile of detected fatty acids differ significantly between all samples. The Mac Ret sample had 142 (±11.3) mg of fatty acids per gram of sample, with principal fatty acids being saturated FAs C4:0 (butyric acid, 115±9.44 mg/g lipid) and C8:0 (caprylic acid, 17.5±1.26 mg/g lipid). Main unsaturated fatty acids in this sample, DHA (C22:6) and palmitoleic acid (C16:1), were both present in concentrations below 1 mg/g lipid (0.74±0.13 and 0.45±0.04 mg/g lipid, respectively). The FAMEs profile of Blue whiting retentate (BW Ret), however, is dominated by unsaturated fatty acids (UFAs), which are present at concentration 220 (±16.0) mg/g lipid in contrast to low concentration of saturated fatty acids (SFA, 57.9±3.79 mg/g lipid). Most abundant unsaturated fatty acids in the BW Ret sample are DHA (C22:6; 111 8.44 mg/g lipid), oleic acid (C18:1 c; 42.8±2.54 mg/g lipid) and EPA (C20:5 n3; 21.6±1.78 mg/g lipid); while stearic acid (C18:0) and myristic acid (C14:0) are the most abundant SFAs (9.68±0.74 and 4.24±0.35 mg/g lipid, respectively). The analysed permeate fractions (Mac 10 KDa and BW 10 KDa) contained only low concentrations of caprilyc acid (in Bw 10 KDa, at 0.49±0.06 mg/g lipid) or combination of caprilyc and capric acid (in Mac 10 KDa, at 105±4.22 and 55.3±1.67 mg/g lipid, respectively).

TABLE 5
Fatty acid profiles of Mackerel and Blue whiting retentate and 10 KDa permeate fractions

| | | Mg fatty acid/g lipid | | | |
|---|---|---|---|---|---|
| Name | Short | Mac | BW | Mac | BW |
| (methyl ester) | Name | Ret | Ret | 10 KDa | 10 KDa |
| Butyric acid | C4:0 | 115 ± 9.44 | | | |
| Caproic acid | C6:0 | | | | |
| Caprylic acid | C8:0 | 17.5 ± 1.26 | | 105 ± 4.22 | 0.49 ± 0.06 |
| Capric acid | C10:0 | 7.68 ± 0.51 | | 55.3 ± 1.67 | |
| Undecanoic acid | C11:0 | | | | |
| Lauric acid | C12:0 | | | | |
| Tridecanoic acid | C13:0 | | | | |
| Myristic acid | C14:0 | | 4.24 ± 0.35 | | |
| Myristoleic acid | C14:1 | | | | |
| Pentadecanoic acid | C15:0 | | 0.29 ± 0.02 | | |
| cis-10-Pentadecenoic acid | C15:1 | | | | |
| Palmitic acid | C16:0 | 1.1 ± 0.14 | 43.5 ± 2.54 | | |
| Palmitoleic acid | C16:1 | 0.45 ± 0.04 | 6.99 ± 0.49 | | |
| Heptadecanoic acid | C17:0 | | | | |
| cis-10-Heptadecenoic acid | C17:1 | | 0.48 ± 0.03 | | |
| Stearic acid | C18:0 | | 9.68 ± 0.74 | | |
| Elaidic acid | C18:1 t | | | | |
| Oleic acid | C18:1 c | | 42.8 ± 2.54 | | |
| Linolelaidic acid | C18:2 t | | 0.16 ± 0.07 | | |
| Linoleic acid | C18:2 c | | 1.87 ± 0.18 | | |
| γ-Linolenic acid | C18:3 n6 | | | | |
| α-Linolenic acid | C18:3 n3 | | 0.58 ± 0.07 | | |
| Arachidic acid | C20:0 | | | | |
| cis-11-Eicosenoic acid | C20:1 | 0.06 ± 0.09 | 16.6 ± 1.37 | | |
| cis-11,14-Eicosadienoic acid | C20:2 | | 0.09 ± 0.009 | | |
| Henicosanoic acid | C21:0 | | | | |
| cis-8,11,14-Eicosatrienoic acid | C20:3 n6 | | | | |
| Arachidonic acid | C20:4 n6 | | 4.12 ± 0.34 | | |
| cis-11,14,17-Eicosatrienoic acid | C20:3 n3 | | | | |
| cis-5,8,11,14,17-Eicosapentaenoic | C20:5 n3 (EPA) | | 21.6 ± 1.78 | | |
| Behenic acid | C22:0 | | 0.19 ± 0.18 | | |
| Erucic acid | C22:1 | | 2.81 ± 0.20 | | |
| cis-13,16-Docosadienoic acid | C22:2 n6 | | | | |
| Tricosanoic acid | C23:0 | | | | |
| Lignoceric acid | C24:0 | | | | |
| cis-4,7,10,13,16,19-Docosahexaenoic acid | C22:6 (DHA) | 0.74 ± 0.13 | 111 ± 8.44 | | |
| Nervonic acid | C24:1 | | 10.9 ± 0.70 | | |
| TFA¹ | | 142 ± 11.3 | 278 ± 19.8 | 160 ± 5.89 | 0.50 ± 0.06 |
| SFA² | | 141 ± 11.1 | 57.9 ± 3.79 | 160 ± 5.89 | 0.50 ± 0.06 |
| UFA³ | | 1.26 ± 0.19 | 220 ± 16.0 | 0 | 0 |
| MUFA⁴ | | 0.51 ± 0.09 | 80.9 ± 5.28 | 0 | 0 |
| PUFA⁵ | | 0.74 ± 0.13 | 139 ± 10.8 | 0 | 0 |

¹Total fatty acids;
²saturated fatty acids;
³unsaturated fatty acids;
⁴monounsaturated fatty acids;
⁵polyunsaturated fatty acids

The total fatty acids (TFA) content of all the fractions analysed ranged between 293+/−27.6 and 981+/−29.4 mg/g. Unsaturated fatty acids (UFAs) contributed between 167.3+/−9.45 and 786+/−22.6 mg/g of the above total whereas polyunsaturated fatty acids (PUFAs) ranged from 75.71+/−6.15 to 479.9+/−8.94 mg/g. All the samples analysed had a higher content of unsaturated fatty acids compared to their saturated counterparts. The whole fraction of the sample collected on the 30/01/20 from the processing of salmon and white fish had the highest content of unsaturated fatty acids (786+/−22.6 mg/g). Polyunsaturated fatty acids have been shown to have beneficial effects on normal health and chronic diseases, by, for example, regulation of immune, lipid levels, and cardiovascular functions. Among the monounsaturated fatty acids (MUFAs), C18:1 cis-9 (oleic acid) was present in higher quantities in all the samples analysed (0-231+/−7.5 mg/g). The permeate fraction of the sample collected on 30/01/20 from the processing of salmon and white fish had the highest quantity (231+/−7.5 mg/g) of oleic acid. Oleic acid has been shown to reduce the levels of low-density lipoprotein in blood which in turn reduces the risk of heart disease and stroke. Other monounsaturated fatty acids, such as C16:1, C18:1 trans, C20:1, and C22:1 were also identified in lower quantities in the range of 0-116 mg/g. In the category of PUFAs, which, were for all but one sample, less in quantity than their MUFAs counterparts, C18:2 cis-9 (linoleic acid) was the dominating fatty acids with quantities of up to 234+/−2.7 mg/g. Linoleic acid is an essential fatty acid that plays an important role in the synthesis of eicosanoids, which are involved in a variety of physiological functions. Other important health-related PUFAs such as omega-3 fatty acids C22:6 (DHA) and C20:5 n3 (EPA) were identified with the levels in the ranges of 1.79+/−0.44 to 59.07+/−1.5 and 1.6+/−0.68 to 34.74+/−1.5 mg/g. Highest quantities of both EPA (34.7 mg/g) and DHA (59.1 mg/g), were identified in the whole fraction of the samples collected on the 30/01/20 from the processing of salmon and white fish.

From the saturated fatty acids group, all the analysed fish blood water fractions contained a significant amount of C14:0, C16:0, C18:0 and C22:0 fatty acids with C16:0 (palmitic acid) dominating this group with the quantities ranging from 26.6+/−0.26 to 133.8+/−3.9 mg/g. This observation is in line with other studies showing high amounts of C16:0 in fish oil. Other odd number saturated fatty acids such as C13:0, C15:0, C21:0 and C23:0 were also identified, albeit, in very low quantities. Interestingly, for almost every sample analysed, the permeate fraction had the highest amounts of fatty acids compared to the whole and retentate fractions. This data may suggest that the fatty acids investigated in this study are <3 kDa in size or are mostly associated with the <3 kDa proteins in the blood water. Based on the above observation, ultrafiltration may be used to concentrate and alter the fatty acid profile of fish blood waters.

Techno-Functional Properties of Blood Water Fractions

Water and Oil Holding Capacities

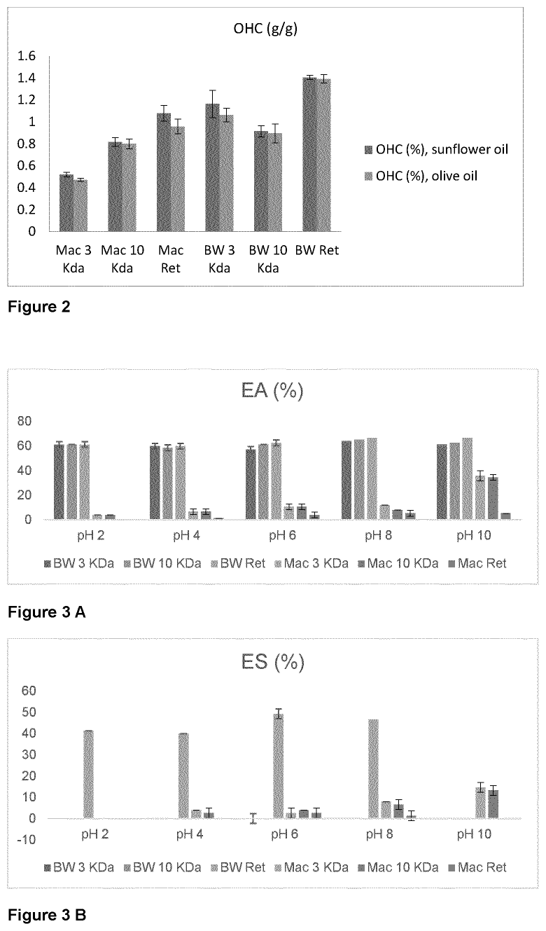

Water holding capacities (WHC) and oil holding capacities (OHC) were determined for all Mackerel and Blue whiting retentate and permeate samples. OHC values (g/g) for sunflower and olive oil are presented in FIG. 2. WHC values could not be determined using the applied methodology, since all of the tested samples were lost in the process of draining the water supernatant. The calculated OHC were in the range between around 0.5 g/g in case of Mac 3 KDa permeate (0.52±0.02 g/g and 0.47±0.02 g/g for sunflower and olive oil, respectively) to around 1.4 g/g for BW Ret sample (1.41±0.02 g/g and 1.39±0.04 g/g for sunflower and olive oil, respectively). Both 10 KDa permeate samples had similar OHC values (0.82±0.04 g/g and 0.80±0.04 g/g for sunflower and olive oil, respectively for Mac 10 KDa and 0.91±0.05 g/g and 0.89±0.09 g/g for sunflower and olive oil, respectively for BW 10 KDa sample).

Emulsifying Capacity and Emulsifying Stability

The emulsifying activity (EA) and emulsifying stability (ES) of 1% (w/v) sample solutions (Mackerel and Blue whiting retentates and permeates) at pH range from 2 to 10 are presented in FIG. 3. EA values of Blue whiting fractions range from 60.0 (±0.00) % in case of BW3 KDa sample at pH=4 to 66.7 (±2.31) % for BW Ret sample at pH=10 and therefore do not differ significantly across the pH range. On the other hand, the EA values of Mackerel samples are significantly lower than Blue whiting samples (ranging from 0.00±0.00% in case of Mac Ret sample at pH=2 to 36.0±4.00% for Mac 3 KDa sample at pH=10). Mackerel fractions also show a dependence on the pH value and have significantly higher EA values at high pH (8 and 10). Most of the tested fractions do not show good ES and complete loss of emulsion layer is noticeable under experimental conditions. All emulsion layers of Blue whiting fractions collapse after heating, except the BW Ret fraction, which maintains a good level of EA (40.0±0.00 to 49.3±2.31%) at all pH values, except at pH=10 where its EA is zero. Mackerel fractions, however, maintained a level of emulsifying activity after heating that ranged from 1.33 (±2.31) % for Mac Ret sample at pH=8 to 14.7 (±2.31) % in case of Mac 3 KDa fraction at pH=10. All Mackerel fractions did not maintain emulsion at pH=2.

Foaming Capacity, Foaming Stability and Solubility

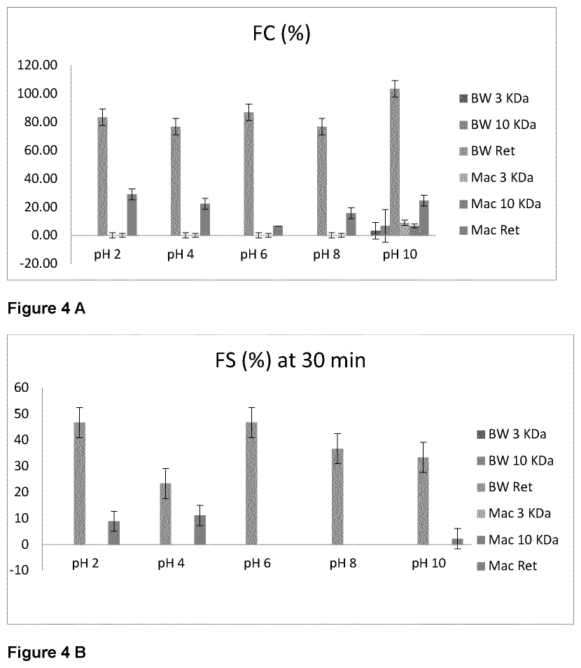

The foaming capacity (FC) and foaming stability (FS) of the 1% (w/v) Mackerel and Blue whiting sample solutions at pH range from 2 to 10 are presented in FIG. 4. The 3 and 10 KDa permeate fractions formed foam only at pH=10, ranging from 3.3 (±5.77) % for BW 10 KDa sample to 8.89 (±3.85) % in the case of Mac 3 KDa fraction. Both BW Ret and Mac Ret samples possessed foaming capacities across the entire tested pH range, with values from 6.67 (±0.00) % for Mac Ret sample at pH=6 to 103 (±5.77) % for BW Ret sample at pH=10. FS of the sample solutions after 30 minutes indicates that none of the 3 KDa and 10 KDa permeate fractions did not retain foaming properties. The retentate fractions on the other hand still had foaming properties, with FC values of Blue whiting samples ranging from 23.3 (±5.77) % at pH=4 to 46.7 (±5.77) % at pH=2 and 6. FC values of Mac Ret samples were significantly lower, between 2.22 (±3.85) % and 11.1 (±3.85) % at pH=10 and 4, respectively. No foaming was retained for Mac Ret sample at pH values at 6 and 8.

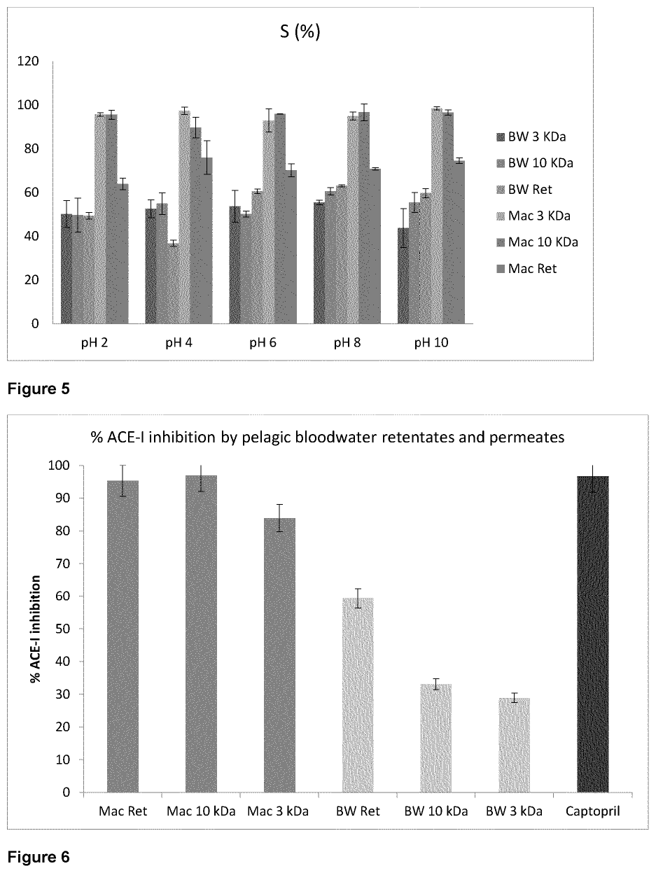

Solubility (S) of 1% (w/v) sample solutions at pH ranges between 2 and 10 is presented in FIG. 5. As can be seen, Blue whiting samples (3 and 10 KDa permeates and retentate) had significantly lower solubilities compared to Mackerel samples across the pH range, with values between 36.9 (±1.42) % (BW Ret at pH=4) and 63.1 (±0.54) % (BW Ret at pH=8); while Mackerel sample solubility ranged between 63.9 (±2.59) % and 98.6 (±0.69) % (Mac Ret at pH=2 and Mac 3 KDa at pH=10, respectively).

Bioactivity Testing

ACE-I and Renin Inhibition Activity

The results of ACE-I inhibition activity of the Blue whiting and Mackerel samples tested at 1 mg/mL (w/v) concentration, with Captopril used as positive control, is presented in FIG. 6.

The activity of Mackerel fractions is significantly different when compared to the tested Blue whiting samples, with Mac 10 KDa and Mac Ret samples having highest inhibition activity (96.9±0.29% and 95.4±0.11%, respectively) and Mac 3 KDa sample with 83.9 (±18.9) % inhibition. The activity of Blue whiting samples was 59.4 (±16.5), 33.1 (±0.59) and 28.9 (±16.4) % for BW Ret, BW 10 KDa and BW 3 KDa samples, respectively.

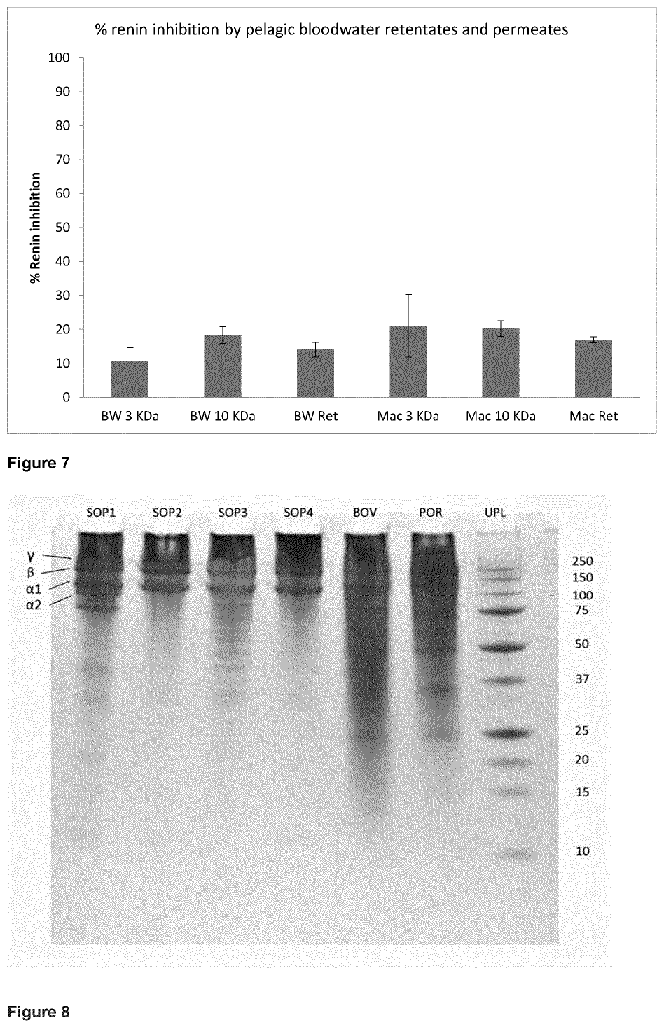

Renin inhibition activity, tested at 1 mg/mL concentration (w/v) of the samples is presented in FIG. 7. The inhibition activity ranged from 10.6 (±16.5) % for BW 3 KDa sample to 21.0 (±9.22) % for Mac 3 KDa sample.

Peptide Identification by Tandem Mass Spectrometry

MS analysis results of the 10 kDa Mackerel and Blue whiting 10 KDa permeate fractions and in Mackerel retentate are presented in Table 6. A total of 65 peptides were identified in the BW 10 KDa sample, 103 peptides were identified in the Mac 10 KDa permeate and 19 peptides in Mac Ret sample. All the identified peptides were identified as novel when checked against the BIOPEP database of known peptides.

| | | | | | |
|---|---|---|---|---|---|
| | | | | Unique | Position |
| | | NCBI | | in | in |
| | Hit | accession | Confirmed in NCBI database | BIOPEP | protein |
| Peptide Sequence¹ | number² | number³ | (corresponding protein) | database | sequence |

BW 10 KDa

| | | | | | |
|---|---|---|---|---|---|
| GFAGDDAPR | 1 | ACTA_BOVIN | Actin, aortic smooth muscle | Y | [22-31] |
| (SEQ ID 1) | | | OS = Bos taurus OX = 9913 | | |
| | | | GN = ACTA2 PE = 1 SV = 1 | | |
| DSYVGDEAQSK | 1 | ACTA_BOVIN | Actin, aortic smooth muscle | Y | [53-64] |
| (SEQ ID 2) | | | OS = Bos taurus OX = 9913 | | |
| | | | GN = ACTA2 PE = 1 SV = 1 | | |
| AGFAGDDAPRAVFPS | 1 | ACTA_BOVIN | Actin, aortic smooth muscle | Y | [21-36] |
| (SEQ ID 3) | | | OS = Bos taurus OX = 9913 | | |
| | | | GN = ACTA2 PE = 1 SV = 1 | | |
| GDDAPRAVFPSIVGRP | 1 | ACTA_BOVIN | Actin, aortic smooth muscle | Y | [25-40] |
| (SEQ ID 4) | | | OS = Bos taurus OX = 9913 | | |
| | | | GN = ACTA2 PE = 1 SV = 1 | | |
| YELPDGQVITIGNER | 1 | ACTA_BOVIN | Actin, aortic smooth muscle | Y | [242-256] |
| (SEQ ID 5) | | | OS = Bos taurus OX = 9913 | | |
| | | | GN = ACTA2 PE = 1 SV = 1 | | |
| SYELPDGQVITIGNER | 1 | ACTA_BOVIN | Actin, aortic smooth muscle | Y | [241-256] |
| (SEQ ID 6) | | | OS = Bos taurus OX = 9913 | | |
| | | | GN = ACTA2 PE = 1 SV = 1 | | |
| VGMGQKDSYVGDEAQS | 1 | ACTA_BOVIN | Actin, aortic smooth muscle | Y | [47-63] |
| K (SEQ ID 7) | | | OS = Bos taurus OX = 9913 | | |
| | | | GN = ACTA2 PE = 1 SV = 1 | | |
| YELPDGQVITIGNERF | 1 | ACTA_BOVIN | Actin, aortic smooth muscle | Y | [242-257] |
| (SEQ ID 8) | | | OS = Bos taurus OX = 9913 | | |
| | | | GN = ACTA2 PE = 1 SV = 1 | | |
| GFAGDDAPRAVFPSIVG | 1 | ACTA_BOVIN | Actin, aortic smooth muscle | Y | [22-40] |
| RP (SEQ ID 9) | | | OS = Bos taurus OX = 9913 | | |
| | | | GN = ACTA2 PE = 1 SV = 1 | | |
| SYELPDGQVITIGNERF | 1 | ACTA_BOVIN | Actin, aortic smooth muscle | Y | [241-257] |
| (SEQ ID 10) | | | OS = Bos taurus OX = 9913 | | |
| | | | GN = ACTA2 PE = 1 SV = 1 | | |
| LRVAPEEHPTLLTEAPLN | 1 | ACTA_BOVIN | Actin, aortic smooth muscle | Y | [96-115] |
| PK (SEQ ID 11) | | | OS = Bos taurus OX = 9913 | | |
| | | | GN = ACTA2 PE = 1 SV = 1 | | |
| HQGVMVGMGQKDSYV | 1 | ACTA_BOVIN | Actin, aortic smooth muscle | Y | [42-63] |
| GDEAQSK (SEQ ID 12) | | | OS = Bos taurus OX = 9913 | | |
| | | | GN = ACTA2 PE = 1 SV = 1 | | |
| RVAPEEHPVLLTEAPLNP | 1 | ACT2_MOLOC | Actin, muscle-type OS = Molgula | Y | [98-116] |
| K (SEQ ID 13) | | | oculata OX = 27575 PE = 3 SV = 1 | | |
| HQGVMVGMGQKDCYV | 1 | ACTBL_HUMAN | Beta-actin-like protein 2 | Y | [41-61] |
| GDEAQS (SEQ ID 14) | | | OS = Homo sapiens OX = 9606 | | |
| | | | GN = ACTBL2 PE = 1 SV = 2 | | |
| DNLEPCMYVCVVAQSK | 1 | SH3B4_SERQU | SH3 domain-binding protein 4 | Y | [446-461] |
| (SEQ ID 15) | | | OS = Seriola quinqueradiata | | |
| | | | OX = 8161 GN = sh3bp4 PE = 2 SV = 1 | | |
| TPNVSVVDLTVR (SEQ | 2 | G3P_DANRE | Glyceraldehyde-3-phosphate | Y | [235-246] |
| ID 16) | | | dehydrogenase OS = Danio rerio | | |
| | | | OX = 7955 GN = gapdh PE = 2 SV = 2 | | |
| VVESTGVFTTIEK (SEQ | 2 | G3P_DANRE | Glyceraldehyde-3-phosphate | Y | [93-105] |
| ID 17) | | | dehydrogenase OS = Danio rerio | | |
| | | | OX = 7955 GN = gapdh PE = 2 SV = 2 | | |
| VPTPNVSVVDLTVR | 2 | G3P_DANRE | Glyceraldehyde-3-phosphate | Y | [233-246] |
| (SEQ ID 18) | | | dehydrogenase OS = Danio rerio | | |
| | | | OX = 7955 GN = gapdh PE = 2 SV = 2 | | |
| FRVPTPNVSVVDLT | 2 | G3P_DANRE | Glyceraldehyde-3-phosphate | Y | [231-244] |
| (SEQ ID 19) | | | dehydrogenase OS = Danio rerio | | |
| | | | OX = 7955 GN = gapdh PE = 2 SV = 2 | | |
| RVPTPNVSVVDLTVR | 2 | G3P_DANRE | Glyceraldehyde-3-phosphate | Y | [232-246] |
| (SEQ ID 20) | | | dehydrogenase OS = Danio rerio | | |
| | | | OX = 7955 GN = gapdh PE = 2 SV = 2 | | |
| GGDDLDPNYV (SEQ ID | 3 | KCRM_TETCF | Creatine kinase M-type | Y | |
| 21) | | | OS = Tetronarce californica | | |
| | | | OX = 7787 PE = 1 SV = 1 | | |
| SIDDLMPAQK (SEQ ID | | KCRM_TETCF | Creatine kinase M-type | Y | |
| 22) | | | OS = Tetronarce californica | | |
| | | | OX = 7787 PE = 1 SV = 1 | | |
| DIIQTGVDNPGHPF (SEQ | | KCRM_TETCF | Creatine kinase M-type | Y | |
| ID 23) | | | OS = Tetronarce californica | | |
| | | | OX = 7787 PE = 1 SV = 1 | | |
| DDIIQTGVDNPGHPF | | KCRM_TETCF | Creatine kinase M-type | Y | |
| (SEQ ID 24) | | | OS = Tetronarce californica | | |
| | | | OX = 7787 PE = 1 SV = 1 | | |
| DDIIQTGVDNPGHPFI | | KCRM_TETCF | Creatine kinase M-type | Y | |
| (SEQ ID 25) | | | OS = Tetronarce californica | | |
| | | | OX = 7787 PE = 1 SV = 1 | | |
| DVIQTGVDNPGHPFI | | KCRB_CHICK | Creatine kinase B-type OS = Gallus | Y | |
| (SEQ ID 26) | | | gallus OX = 9031 GN = CKB PE = 1 | | |
| | | | SV = 1 | | |
| DDVIQTGVDNPGHPF | | KCRB_CHICK | Creatine kinase B-type OS = Gallus | Y | |
| (SEQ ID 27) | | | gallus OX = 9031 GN = CKB PE = 1 | | |
| | | | SV = 1 | | |
| TLDDVIQTGVDNPGHP | | KCRB_CHICK | Creatine kinase B-type OS = Gallus | Y | |
| (SEQ ID 28) | | | gallus OX = 9031 GN = CKB PE = 1 | | |
| | | | SV = 1 | | |
| LDDVIQTGVDNPGHPF | | KCRB_CHICK | Creatine kinase B-type OS = Gallus | Y | |
| (SEQ ID 29) | | | gallus OX = 9031 GN = CKB PE = 1 | | |
| | | | SV = 1 | | |
| DDVIQTGVDNPGHPFI | | KCRB_CHICK | Creatine kinase B-type OS = Gallus | Y | |
| (SEQ ID 30) | | | gallus OX = 9031 GN = CKB PE = 1 | | |
| | | | SV = 1 | | |
| TLDDVIQTGVDNPGHPF | | KCRB_CHICK | Creatine kinase B-type OS = Gallus | Y | |
| (SEQ ID 31) | | | gallus OX = 9031 GN = CKB PE = 1 | | |
| | | | SV = 1 | | |
| SIDDMIPAQK (SEQ ID | | KCRM_RABIT | Creatine kinase M-type | Y | |
| 32) | | | OS = Oryctolagus cuniculus | | |
| | | | OX = 9986 GN = CKM PE = 1 SV = 1 | | |
| ISGLIYEETR (SEQ | | H4_BOVIN | Histone H4 OS = Bos taurus | Y | |
| ID 33) | | | OX = 9913 PE = 1 SV = 2 | | |
| TVTAMDVVYALK (SEQ | | H4_BOVIN | Histone H4 OS = Bos taurus | Y | |
| ID 34) | | | OX = 9913 PE = 1 SV = 2 | | |
| DNIQGITKPAIR (SEQ | | H4_BOVIN | Histone H4 OS = Bos taurus | Y | |
| ID 35) | | | OX = 9913 PE = 1 SV = 2 | | |
| GLAGVENVTELK (SEQ | | PYGM_BOVIN | Glycogen phosphorylase, muscle | Y | |
| ID 36) | | | form OS = Bos taurus OX = 9913 | | |
| | | | GN = PYGM PE = 1 SV = 3 | | |
| VSLSLHADWAEPA (SEQ | | KLOTB_HUMAN | Beta-klotho OS = Homo sapiens | Y | |
| ID 37) | | | OX = 9606 GN = KLB PE = 1 SV = 1 | | |
| TYAALSVLHSEK (SEQ | | HBB1_ARCGL | Hemoglobin subunit beta-1 | Y | |
| ID 38) | | | (Fragment) OS = Arctogadus | | |
| | | | glacialis OX = 185735 GN = hbb1 | | |
| | | | PE = 1 SV = 1 | | |
| HVFGESDELIGQK (SEQ | | TPISA_DANRE | Triosephosphate isomerase A | Y | |
| ID 39) | | | OS = Danio rerio OX = 7955 | | |
| | | | GN = tpila PE = 2 SV = 1 | | |
| EEVNALT (SEQ ID 40) | | CCND2_RAT | G1/S-specific cyclin-D2 OS = Rattus | Y | |
| | | | norvegicus OX = 10116 GN = Ccnd2 | | |
| | | | PE = 1 SV = 1 | | |
| PGVTVKDVNQQEFV | | RS19_PAGMA | 40S ribosomal protein S19 | Y | |
| (SEQ ID 41) | | | OS = Pagrus major OX = 143350 | | |
| | | | GN = rps19 PE = 2 SV = 1 | | |
| AVSEGTKAVTKYTSS | | H2B1A_MOUSE | Histone H2B type 1-A OS = Mus | Y | |
| (SEQ ID 42) | | | musculus OX = 10090 | | |
| | | | GN = Hist1h2ba PE = 1 SV = 3 | | |
| HAVSEGTKAVTKYTSS | | H2B1A_MOUSE | Histone H2B type 1-A OS = Mus | Y | |
| (SEQ ID 43) | | | musculus OX = 10090 | | |
| | | | GN = Hist1h2ba PE = 1 SV = 3 | | |
| PDINVDVALTMPREILQ | | NOL6_HUMAN | Nucleolar protein 6 OS = Homo | Y | |
| (SEQ ID 44) | | | sapiens OX = 9606 GN = NOL6 PE = 1 | | |
| | | | SV = 2 | | |
| DIGAEALSRLVAVYPQTK | | HBA1_ARCGL | Hemoglobin subunit alpha-1 | Y | |
| (SEQ ID 45) | | | OS = Arctogadus glacialis | | |
| | | | OX = 185735 GN = hba1 PE = 1 SV = 3 | | |
| DVYVNDAFGTAH (SEQ | | PGK_CHICK | Phosphoglycerate kinase | Y | |
| ID 46) | | | OS = Gallus gallus OX = 9031 | | |
| | | | GN = PGK PE = 2 SV = 2 | | |
| VERPEEGELSSGPAQLVG | | IGSF8_MOUSE | Immunoglobulin superfamily | Y | |
| GVGQD (SEQ ID 47) | | | member 8 OS = Mus musculus | | |
| | | | OX = 10090 GN = Igsf8 PE = 1 | | |
| | | | SV = 2 | | |
| AKVAVLGASGGIGQPLSL | | MDHM_BOVIN | Malate dehydrogenase, | Y | |
| LLK (SEQ ID 48) | | | mitochondrial OS = Bos taurus | | |
| | | | OX = 9913 GN = MDH2 PE = 1 SV = 1 | | |
| VHDAILEDLVFPSEIVG | | RS7_BOVIN | 40S ribosomal protein S7 OS = Bos | Y | |
| (SEQ ID 49) | | | taurus OX = 9913 GN = RPS7 PE = 2 | | |
| | | | SV = 1 | | |
| KQTAEMYVKADGSVEEA | | SYNE1_HUMAN | Nesprin-1 OS = Homo sapiens | Y | |
| (SEQ ID 50) | | | OX = 9606 GN = SYNE1 PE = 1 SV = 4 | | |
| VLNASVIPEGQFIDNK | | MYSS_CYPCA | Myosin heavy chain, fast skeletal | Y | |
| (SEQ ID 51) | | | muscle OS = Cyprinus carpio | | |
| | | | OX = 7962 PE = 2 SV = 2 | | |
| GRNKADNLDVP (SEQ ID | | DTWD1_MOUSE | DTW domain-containing protein | Y | |
| 52) | | | 1 OS = Mus musculus OX = 10090 | | |
| | | | GN = Dtwd1 PE = 2 SV = 1 | | |
| NGQLAASVETAGDSELFL | | FSCN1_HUMAN | Fascin OS = Homo sapiens | Y | |
| (SEQ ID 53) | | | OX = 9606 GN = FSCN1 PE = 1 SV = 3 | | |
| HLQLAVRNDEELN (SEQ | | H2A2B_MOUSE | Histone H2A type 2-B OS = Mus | Y | |
| ID 54) | | | musculus OX = 10090 | | |
| | | | GN = Hist2h2ab PE = 1 SV = 3 | | |
| AVRNDEELNKLLGGVTIA | | H2A2B_MOUSE | Histone H2A type 2-B OS = Mus | Y | |
| QGGVLPN (SEQ ID 55) | | | musculus OX = 10090 | | |
| | | | GN = Hist2h2ab PE = 1 SV = 3 | | |
| TAGVPGFVLDTNASVSN | | SPIT1_HUMAN | Kunitz-type protease inhibitor 1 | Y | |
| (SEQ ID 56) | | | OS = Homo sapiens OX = 9606 | | |
| | | | GN = SPINT1 PE = 1 SV = 2 | | |
| AAVPSGASTGIYEALELR | | ENOA_ALLM | Alpha-enolase OS = Alligator | Y | |
| DNDK (SEQ ID 57) | | | mississippiensis OX = 8496 PE = 2 | | |
| | | | SV = 3 | | |
| VENMGRVNYGAY (SEQ | | BGAL_HUMAN | Beta-galactosidase OS = Homo | Y | |
| ID 58) | | | sapiens OX = 9606 GN = GLB1 PE = 1 | | |
| | | | SV = 2 | | |
| KKNEVATEECS (SEQ ID | | SENP8_MOUSE | Sentrin-specific protease 8 | Y | |
| 59) | | | OS = Mus musculus OX = 10090 | | |
| | | | GN = Senp8 PE = 1 SV = 2 | | |
| KSNGPNGNE (SEQ ID | | BRNP3_HUMAN | BMP/retinoic acid-inducible | Y | |
| 60) | | | neural-specific protein 3 | | |
| | | | OS = Homo sapiens OX = 9606 | | |
| | | | GN = BRINP3 PE = 1 SV = 1 | | |
| INMQGQVVQT (SEQ ID | | CLOCK_TYTAL | Circadian locomoter output | Y | |
| 61) | | | cycles protein kaput OS = Tyto | | |
| | | | alba OX = 56313 GN = CLOCK PE = 2 | | |
| | | | SV = 1 | | |
| YDVAGQG (SEQ ID 62) | | P2R3C_BOVIN | Serine/threonine-protein | Y | |
| | | | phosphatase 2A regulatory | | |
| | | | subunit B^{~~} subunit gamma | | |
| | | | OS = Bos taurus OX = 9913 | | |
| | | | GN = PPP2R3C PE = 2 SV = 1 | | |
| KPCSSQCYDTKTPDPSVD | | AR13A_MOUSE | ADP-ribosylation factor-like | Y | |
| VSL (SEQ ID 63) | | | protein 13A OS = Mus musculus | | |
| | | | OX = 10090 GN = Arl13a PE = 2 | | |
| | | | SV = 1 | | |
| DEELDSLIKATIAGGGVIP | | H2AV_BOVIN | Histone H2A.V OS = Bos taurus | Y | |
| H (SEQ ID 64) | | | OX = 9913 GN = H2AFV PE = 2 SV = 3 | | |

Mac 10 KDa

| | | | | | |
|---|---|---|---|---|---|
| KLLGGVTIAQGGVLPNIQ | 1 | H2A2B_MOUSE | Histone H2A type 2-B OS = Mus | Y | [96-113] |
| (SEQ ID 65) | | | musculus OX = 10090 | | |
| | | | GN = Hist2h2ab PE = 1 SV = 3 | | |
| LTAEILELAGNAARDNK | 1 | H2A2B_MOUSE | Histone H2A type 2-B OS = Mus | Y | [59-75] |
| (SEQ ID 66) | | | musculus OX = 10090 | | |
| | | | GN = Hist2h2ab PE = 1 SV = 3 | | |
| LLGGVTIAQGGVLPNIQA | 1 | H2A2B_MOUSE | Histone H2A type 2-B OS = Mus | Y | [97-118] |
| VLLP (SEQ ID 67) | | | musculus OX = 10090 | | |
| | | | GN = Hist2h2ab PE = 1 SV = 3 | | |
| LLGGVTIAQGGVLPNIQA | 1 | H2A2B_MOUSE | Histone H2A type 2-B OS = Mus | Y | [97-119] |
| VLLPK (SEQ ID 68) | | | musculus OX = 10090 | | |
| | | | GN = Hist2h2ab PE = 1 SV = 3 | | |
| AVLEYLTAEILELAGNAA | 1 | H2A2B_MOUSE | Histone H2A type 2-B OS = Mus | Y | [54-75] |
| RDNK (SEQ ID 69) | | | musculus OX = 10090 | | |
| | | | GN = Hist2h2ab PE = 1 SV = 3 | | |
| RNDEELNKLLGGVTIAQ | 1 | H2A2B_MOUSE | Histone H2A type 2-B OS = Mus | Y | [89-114] |
| GGVLPNIQA (SEQ ID | | | musculus OX = 10090 | | |
| 70) | | | GN = Hist2h2ab PE = 1 SV = 3 | | |
| AVRNDEELNKLLGGVTIA | 1 | H2A2B_MOUSE | Histone H2A type 2-B OS = Mus | Y | [87-113] |
| QGGVLPNIQ (SEQ ID | | | musculus OX = 10090 | | |
| 71) | | | GN = Hist2h2ab PE = 1 SV = 3 | | |
| AVRNDEELNKLLGGVTIA | 1 | H2A2B_MOUSE | Histone H2A type 2-B OS = Mus | Y | [87-114] |
| QGGVLPNIQA (SEQ ID | | | musculus OX = 10090 | | |
| 72) | | | GN = Hist2h2ab PE = 1 SV = 3 | | |
| LAVRNDEELNKLLGGVTI | 1 | H2A2B_MOUSE | Histone H2A type 2-B OS = Mus | Y | [86-113] |
| AQGGVLPNIQ (SEQ ID | | | musculus OX = 10090 | | |
| 73) | | | GN = Hist2h2ab PE = 1 SV = 3 | | |
| AVRNDEELNKLLGGVTIA | 1 | H2A2B_MOUSE | Histone H2A type 2-B OS = Mus | Y | [87-115] |
| QGGVLPNIQAV (SEQ ID | | | musculus OX = 10090 | | |
| 74) | | | GN = Hist2h2ab PE = 1 SV = 3 | | |
| AVGKVIPELNGK (SEQ | 2 | G3P_DANRE | Glyceraldehyde-3-phosphate | Y | [214-225] |
| ID 75) | | | dehydrogenase OS = Danio rerio | | |
| | | | OX = 7955 GN = gapdh PE = 2 SV = 2 | | |
| RVPTPNVSVVDL (SEQ | 2 | G3P_DANRE | Glyceraldehyde-3-phosphate | Y | [232-243] |
| ID 76) | | | dehydrogenase OS = Danio rerio | | |
| | | | OX = 7955 GN = gapdh PE = 2 SV = 2 | | |
| RVPTPNVSVVDLT (SEQ | 2 | G3P_DANRE | Glyceraldehyde-3-phosphate | Y | [232-244] |
| ID 77) | | | dehydrogenase OS = Danio rerio | | |
| | | | OX = 7955 GN = gapdh PE = 2 SV = 2 | | |
| VVESTGVFTTIEK (SEQ | 2 | G3P_DANRE | Glyceraldehyde-3-phosphate | Y | [93-105] |
| ID 78) | | | dehydrogenase OS = Danio rerio | | |
| | | | OX = 7955 GN = gapdh PE = 2 SV = 2 | | |
| RVPTPNVSVVDLTV | 2 | G3P_DANRE | Glyceraldehyde-3-phosphate | Y | [232-245] |
| (SEQ ID 79) | | | dehydrogenase OS = Danio rerio | | |
| | | | OX = 7955 GN = gapdh PE = 2 SV = 2 | | |
| VPTPNVSVVDLTVR (SEQ | 2 | G3P_DANRE | Glyceraldehyde-3-phosphate | Y | [233-246] |
| ID 80) | | | dehydrogenase OS = Danio rerio | | |
| | | | OX = 7955 GN = gapdh PE = 2 SV = 2 | | |
| TTIEKASAHIKGGAK | 2 | G3P_DANRE | Glyceraldehyde-3-phosphate | Y | [101-115] |
| (SEQ ID 81) | | | dehydrogenase OS = Danio rerio | | |
| | | | OX = 7955 GN = gapdh PE = 2 SV = 2 | | |
| KAVGKVIPELNGKLT | 2 | G3P_DANRE | Glyceraldehyde-3-phosphate | Y | [213-227] |
| (SEQ ID 82) | | | dehydrogenase OS = Danio rerio | | |
| | | | OX = 7955 GN = gapdh PE = 2 SV = 2 | | |
| YVVESTGVFTTIEK (SEQ | 2 | G3P_DANRE | Glyceraldehyde-3-phosphate | Y | [92-105] |
| ID 83) | | | dehydrogenase OS = Danio rerio | | |
| | | | OX = 7955 GN = gapdh PE = 2 SV = 2 | | |
| RVIISAPSADAPMFV | 2 | G3P_DANRE | Glyceraldehyde-3-phosphate | Y | [116-130] |
| (SEQ ID 84) | | | dehydrogenase OS = Danio rerio | | |
| | | | OX = 7955 GN = gapdh PE = 2 SV = 2 | | |
| KAVGKVIPELNGKLTG | 2 | G3P_DANRE | Glyceraldehyde-3-phosphate | Y | [213-228] |
| (SEQ ID 85) | | | dehydrogenase OS = Danio rerio | | |
| | | | OX = 7955 GN = gapdh PE = 2 SV = 2 | | |
| DGRGASQNIIPASTGAAK | 2 | G3P_DANRE | Glyceraldehyde-3-phosphate | Y | [196-213] |
| (SEQ ID 86) | | | dehydrogenase OS = Danio rerio | | |
| | | | OX = 7955 GN = gapdh PE = 2 SV = 2 | | |
| RVIISAPSADAPMFVMG | 2 | G3P_DANRE | Glyceraldehyde-3-phosphate | Y | [116-132] |
| (SEQ ID 87) | | | dehydrogenase OS = Danio rerio | | |
| | | | OX = 7955 GN = gapdh PE = 2 SV = 2 | | |
| GVFTTIEKASAHIKGGAK | 2 | G3P_DANRE | Glyceraldehyde-3-phosphate | Y | [98-115] |
| (SEQ ID 88) | | | dehydrogenase OS = Danio rerio | | |
| | | | OX = 7955 GN = gapdh PE = 2 SV = 2 | | |
| SIFDAGAGIALNDHFVKL | 2 | G3P_DANRE | Glyceraldehyde-3-phosphate | Y | [291-308] |
| (SEQ ID 89) | | | dehydrogenase OS = Danio rerio | | |
| | | | OX = 7955 GN = gapdh PE = 2 SV = 2 | | |
| VIISAPSADAPMFVMGV | 2 | G3P_DANRE | Glyceraldehyde-3-phosphate | Y | [117-137] |
| NHEK (SEQ ID 90) | | | dehydrogenase OS = Danio rerio | | |
| | | | OX = 7955 GN = gapdh PE = 2 SV = 2 | | |
| VVSTDFNGDTH (SEQ ID | 2 | G3P_XENLA | Glyceraldehyde-3-phosphate | Y | |
| 91) | | | dehydrogenase OS = Xenopus | | |
| | | | laevis OX = 8355 GN = gapdh PE = 2 | | |
| | | | SV = 2 | | |
| VVSTDFNGDTHSS (SEQ | 2 | G3P_XENLA | Glyceraldehyde-3-phosphate | Y | |
| ID 92) | | | dehydrogenase OS = Xenopus | | |
| | | | laevis OX = 8355 GN = gapdh PE = 2 | | |
| | | | SV = 2 | | |
| KAVGKVIPELNGKIT | 2 | G3P_XENLA | Glyceraldehyde-3-phosphate | Y | [213-227] |
| (SEQ ID 93) | | | dehydrogenase OS = Xenopus | | |
| | | | laevis OX = 8355 GN = gapdh PE = 2 | | |
| | | | SV = 2 | | |
| KAVGKVIPELNGKITG | 2 | G3P_XENLA | Glyceraldehyde-3-phosphate | Y | [213-228] |
| (SEQ ID 94) | | | dehydrogenase OS = Xenopus | | |
| | | | laevis OX = 8355 GN = gapdh PE = 2 | | |
| | | | SV = 2 | | |
| KAVGKVIPDLKGKLTG | 2 | G3PT_BOVIN | Glyceraldehyde-3-phosphate | Y | [274-287] |
| (SEQ ID 95) | | | dehydrogenase, testis-specific | | |
| | | | OS = Bos taurus OX = 9913 | | |
| | | | GN = GAPDHS PE = 2 SV = 1 | | |
| VLSGGTTMYPGIADR | | ACTB_OREMO | Actin, cytoplasmic 1 | Y | |
| (SEQ ID 96) | | | OS = Oreochromis mossambicus | | |
| | | | OX = 8127 GN = actb PE = 2 SV = 1 | | |
| YELPDGQVITIGNER | | ACTB_OREMO | Actin, cytoplasmic 1 | Y | |
| (SEQ ID 97) | | | OS = Oreochromis mossambicus | | |
| | | | OX = 8127 GN = actb PE = 2 SV = 1 | | |
| SYELPDGQVITIGNER | | ACTB_OREMO | Actin, cytoplasmic 1 | Y | |
| (SEQ ID 98) | | | OS = Oreochromis mossambicus | | |
| | | | OX = 8127 GN = actb PE = 2 SV = 1 | | |
| FAGDDAPRAVFPSIVGRP | | ACTB_OREMO | Actin, cytoplasmic 1 | Y | |
| (SEQ ID 99) | | | OS = Oreochromis mossambicus | | |
| | | | OX = 8127 GN = actb PE = 2 SV = 1 | | |
| AGFAGDDAPRAVFPSIV | | ACTB_OREMO | Actin, cytoplasmic 1 | Y | |
| GRP (SEQ ID 100) | | | OS = Oreochromis mossambicus | | |
| | | | OX = 8127 GN = actb PE = 2 SV = 1 | | |
| AGFAGDDAPRAVFPSIV | | ACTB_OREMO | Actin, cytoplasmic 1 | Y | |
| GRPR (SEQ ID 101) | | | OS = Oreochromis mossambicus | | |
| | | | OX = 8127 GN = actb PE = 2 SV = 1 | | |
| DLYANTVLSGGTTMYPG | | ACTB_OREMO | Actin, cytoplasmic 1 | Y | |
| IADR (SEQ ID 102) | | | OS = Oreochromis mossambicus | | |
| | | | OX = 8127 GN = actb PE = 2 SV = 1 | | |
| LRVAPEEHPVLLTEAPLN | | ACTB_OREMO | Actin, cytoplasmic 1 | Y | |
| PK (SEQ ID 103) | | | OS = Oreochromis mossambicus | | |
| | | | OX = 8127 GN = actb PE = 2 SV = 1 | | |
| VAPEEHPTLLTEAPLNPK | | ACT2_XENLA | Actin, alpha skeletal muscle 2 | Y | |
| (SEQ ID 104) | | | OS = Xenopus laevis OX = 8355 | | |
| | | | GN = act2 PE = 2 SV = 1 | | |
| VIQTGVDNPGHPF (SEQ | | KCRM_CANLF | Creatine kinase M-type OS = Canis | Y | |
| ID 105) | | | lupus familiaris OX = 9615 | | |
| | | | GN = CKM PE = 1 SV = 3 | | |
| DVIQTGVDNPGHPF | | KCRM_CANLF | Creatine kinase M-type OS = Canis | Y | |
| (SEQ ID 106) | | | lupus familiaris OX = 9615 | | |
| | | | GN = CKM PE = 1 SV = 3 | | |
| TLDDVIQTGVDNPGHPF | | KCRM_CANLF | Creatine kinase M-type OS = Canis | Y | |
| (SEQ ID 107) | | | lupus familiaris OX = 9615 | | |
| | | | GN = CKM PE = 1 SV = 3 | | |
| TLDDVIQTGVDNPGHPFI | | KCRM_CANLF | Creatine kinase M-type OS = Canis | Y | |
| (SEQ ID 108) | | | lupus familiaris OX = 9615 | | |
| | | | GN = CKM PE = 1 SV = 3 | | |
| TVDDVIQTGVDNPGHP | | KCRM_HUMAN | Creatine kinase M-type | Y | |
| (SEQ ID 109) | | | OS = Homo sapiens OX = 9606 | | |
| | | | GN = CKM PE = 1 SV = 2 | | |
| DLEPTVIDEVRTG (SEQ | | TBA1A_CRIGR | Tubulin alpha-1A chain | Y | |
| ID 110) | | | OS = Cricetulus griseus OX = 10029 | | |
| | | | GN = TUBA1A PE = 2 SV = 1 | | |
| VDLEPTVIDEVRTGT | | TBA1A_CRIGR | Tubulin alpha-1A chain | Y | |
| (SEQ ID 111) | | | OS = Cricetulus griseus OX = 10029 | | |
| | | | GN = TUBA1A PE = 2 SV = 1 | | |
| FVDLEPTVIDEVRTGT | | TBA1A_CRIGR | Tubulin alpha-1A chain | Y | |
| (SEQ ID 112) | | | OS = Cricetulus griseus OX = 10029 | | |
| | | | GN = TUBA1A PE = 2 SV = 1 | | |
| GVKVLHGLDRAV (SEQ | | HBB_THUTH | Hemoglobin subunit beta | Y | |
| ID113) | | | OS = Thunnus thynnus OX = 8237 | | |
| | | | GN = hbb PE = 1 SV = 1 | | |
| IAAHGVKVLHGLD (SEQ | | HBB_THUTH | Hemoglobin subunit beta | Y | |
| ID 114) | | | OS = Thunnus thynnus OX = 8237 | | |
| | | | GN = hbb PE = 1 SV = 1 | | |
| AAHGVKVLHGLDR (SEQ | | HBB_THUTH | Hemoglobin subunit beta | Y | |
| ID 115) | | | OS = Thunnus thynnus OX = 8237 | | |
| | | | GN = hbb PE = 1 SV = 1 | | |
| IAAHGVKVLHGLDR | | HBB_THUTH | Hemoglobin subunit beta | Y | |
| (SEQ ID 116) | | | OS = Thunnus thynnus OX = 8237 | | |
| | | | GN = hbb PE = 1 SV = 1 | | |
| DAVSKIDDLVGGLAA | | HBA_CYPCA | Hemoglobin subunit alpha | Y | |
| (SEQ ID 117) | | | OS = Cyprinus carpio OX = 7962 | | |
| | | | GN = hba PE = 1 SV = 3 | | |
| AVGDAVSKIDDLVGGLA | | HBA_CYPCA | Hemoglobin subunit alpha | Y | |
| A (SEQ ID 118) | | | OS = Cyprinus carpio OX = 7962 | | |
| | | | GN = hba PE = 1 SV = 3 | | |
| GAVGDAVSKIDDLVGGL | | HBA_CYPCA | Hemoglobin subunit alpha | Y | |
| AA (SEQ ID 119) | | | OS = Cyprinus carpio OX = 7962 | | |
| | | | GN = hba PE = 1 SV = 3 | | |
| SRIIDTLDIGPA (SEQ | | MATN3_HUMAN | Matrilin-3 OS = Homo sapiens | Y | |
| ID 120) | | | OX = 9606 GN = MATN3 PE = 1 SV = 2 | | |
| RLLLPGELA (SEQ ID | | H2B1A_MOUSE | Histone H2B type 1-A OS = Mus | Y | |
| 121) | | | musculus OX = 10090 | | |
| | | | GN = Hist1h2ba PE = 1 SV = 3 | | |
| HAVSEGTKAVTKYTSS | | H2B1A_MOUSE | Histone H2B type 1-A OS = Mus | Y | |
| (SEQ ID 122) | | | musculus OX = 10090 | | |
| | | | GN = Hist1h2ba PE = 1 SV = 3 | | |
| HAVSEGTKAVTKYTSSK | | H2B1A_MOUSE | Histone H2B type 1-A OS = Mus | Y | |
| (SEQ ID 123) | | | musculus OX = 10090 | | |
| | | | GN = Hist1h2ba PE = 1 SV = 3 | | |
| AFPFLTPEQKKELSDIAHK | | ALDOA_THUAL | Fructose-bisphosphate aldolase A | Y | |
| (SEQ ID 124) | | | (Fragment) OS = Thunnus | | |
| | | | albacares OX = 8236 GN = ALDOA | | |
| | | | PE = 1 SV = 1 | | |
| TVTAMDVVYALK (SEQ | | H4_BOVIN | Histone H4 OS = Bos taurus | Y | |
| ID 125) | | | OX = 9913 PE = 1 SV = 2 | | |
| NIIHGSDTLENA (SEQ | | NDKB_MACMG | Nucleoside diphosphate kinase B | Y | |
| ID 126) | | | (Fragments) OS = Macruronus | | |
| | | | magellanicus OX = 92050 | | |
| | | | GN = nme2 PE = 1 SV = 1 | | |
| WEQLDPSGR (SEQ ID | | PE2R3_BOVIN | Prostaglandin E2 receptor EP3 | Y | |
| 127) | | | subtype OS = Bos taurus OX = 9913 | | |
| | | | GN = PTGER3 PE = 2 SV = 1 | | |
| KKALAAGGYDVDKN | | H1B_XENLA | Histone H1B OS = Xenopus laevis | Y | |
| (SEQ ID 128) | | | OX = 8355 PE = 3 SV = 2 | | |
| LVVYPQTKT (SEQ ID | | HBAE_ORYLA | Hemoglobin embryonic subunit | Y | |
| 129) | | | alpha OS = Oryzias latipes | | |
| | | | OX = 8090 PE = 2 SV = 3 | | |
| VVYPQTKTY (SEQ ID | | HBAE_ORYLA | Hemoglobin embryonic subunit | Y | |
| 130) | | | alpha OS = Oryzias latipes | | |
| | | | OX = 8090 PE = 2 SV = 3 | | |
| LVVYPQTKTY (SEQ ID | | HBAE_ORYLA | Hemoglobin embryonic subunit | Y | |
| 131) | | | alpha OS = Oryzias latipes | | |
| | | | OX = 8090 PE = 2 SV = 3 | | |
| IGADALGRMLVVYPQT | | HBAE_ORYLA | Hemoglobin embryonic subunit | Y | |
| (SEQ ID 132) | | | alpha OS = Oryzias latipes | | |
| | | | OX = 8090 PE = 2 SV = 3 | | |
| KGTLVQTKGTGASGSFK | | H1_ONCMY | Histone H1 OS = Oncorhynchus | Y | |
| (SEQ ID 133) | | | mykiss OX = 8022 PE = 1 SV = 2 | | |
| SLVTKGTLVQTKGTGAS | | H1_ONCMY | Histone H1 OS = Oncorhynchus | Y | |
| GSFK (SEQ ID 134) | | | mykiss OX = 8022 PE = 1 SV = 2 | | |
| IGRIEDVTPIPSDST | | RS14_CRIGR | 40S ribosomal protein S14 | Y | |
| (SEQ ID 135) | | | OS = Cricetulus griseus OX = 10029 | | |
| | | | GN = RPS14 PE = 2 SV = 3 | | |
| DTGAPIRIPVGPETL | | ATPB_CYPCA | ATP synthase subunit beta, | Y | |
| (SEQ ID 136) | | | mitochondrial OS = Cyprinus | | |
| | | | carpio OX = 7962 GN = ATP5F1B | | |
| | | | PE = 2 SV = 1 | | |
| KVLDTGAPIRIPVGPETL | | ATPB_CYPCA | ATP synthase subunit beta, | Y | |
| (SEQ ID 137) | | | mitochondrial OS = Cyprinus | | |
| | | | carpio OX = 7962 GN = ATP5F1B | | |
| | | | PE = 2 SV = 1 | | |
| HAEVRAGAVSALAKFGA | | COPG1_BOVIN | Coatomer subunit gamma-1 | Y | |
| QN (SEQ ID 138) | | | OS = Bos taurus OX = 9913 | | |
| | | | GN = COPG1 PE = 1 SV = 1 | | |
| LAILENANVLAR (SEQ | | ALDOC_HUMAN | Fructose-bisphosphate aldolase C | Y | |
| ID 139) | | | OS = Homo sapiens OX = 9606 | | |
| | | | GN = ALDOC PE = 1 SV = 2 | | |
| IVEPEILPDGDHDL (SEQ | | ALDOC_HUMAN | Fructose-bisphosphate aldolase C | Y | |
| ID 140) | | | OS = Homo sapiens OX = 9606 | | |
| | | | GN = ALDOC PE = 1 SV = 2 | | |
| RTVPPAVTGVTFL (SEQ | | ALDOA_MOUSE | Fructose-bisphosphate aldolase A | Y | |
| ID 141) | | | OS = Mus musculus OX = 10090 | | |
| | | | GN = Aldoa PE = 1 SV = 2 | | |
| LTAEVLELAGNASKDLKV | | H2AV_BOVIN | Histone H2A.V OS = Bos taurus | Y | |
| K (SEQ ID 142) | | | OX = 9913 GN = H2AFV PE = 2 SV = 3 | | |
| RGDEELDSLIKATIAGGG | | H2AV_BOVIN | Histone H2A.V OS = Bos taurus | Y | |
| VIPHIHK | | | OX = 9913 GN = H2AFV PE = 2 SV = 3 | | |
| (SEQ ID 143) | | | | | |
| KLASPMKPVPGTPPS | | MED1_HUMAN | Mediator of RNA polymerase II | Y | |
| (SEQ ID 144) | | | transcription subunit 1 OS = Homo | | |
| | | | sapiens OX = 9606 GN = MED1 PE = 1 | | |
| | | | SV = 4 | | |
| AAVPSGASTGVHEAL | | ENOB_SALSA | Beta-enolase OS = Salmo salar | Y | |
| (SEQ ID 145) | | | OX = 8030 GN = ENO3 PE = 1 SV = 1 | | |
| ENCFWIKVNENKYENVD | | DIAP3_HUMAN | Protein diaphanous homolog 3 | Y | |
| LLCKL (SEQ ID 146) | | | OS = Homo sapiens OX = 9606 | | |
| | | | GN = DIAPH3 PE = 1 SV = 4 | | |
| YFSHWPETTPGSAPVKK | | HBAA_GYMUN | Hemoglobin anodic subunit alpha | Y | |
| (SEQ ID 147) | | | OS = Gymnothorax unicolor | | |
| | | | OX = 296138 PE = 1 SV = 1 | | |
| VPSPKVSDTVVEPY (SEQ | | TBB_PIG | Tubulin beta chain OS = Sus scrofa | Y | |
| ID 148) | | | OX = 9823 PE = 1 SV = 1 | | |
| IDYEQGGVLEV (SEQ ID | | ENTP5_CHICK | Ectonucleoside triphosphate | Y | |
| 149) | | | diphosphohydrolase 5 OS = Gallus | | |
| | | | gallus OX = 9031 GN = ENTPD5 PE = 3 | | |
| | | | SV = 1 | | |
| AETQYEEQIAKI (SEQ | | CCD73_MOUSE | Coiled-coil domain-containing | Y | |
| ID 150) | | | protein 73 OS = Mus musculus | | |
| | | | OX = 10090 GN = Ccdc73 PE = 2 | | |
| | | | SV = 2 | | |
| FGAVGVGSSLTSFGAE | | L14AA_XENLA | Protein LSM14 homolog A-A | Y | |
| (SEQ ID 151) | | | OS = Xenopus laevis OX = 8355 | | |
| | | | GN = Ism14a-a PE = 1 SV = 1 | | |
| AVVGVVAGGGRIDKPI | | RL8_BOVIN | 60S ribosomal protein L8 OS = Bos | Y | |
| (SEQ ID 152) | | | taurus OX = 9913 GN = RPL8 PE = 2 | | |
| | | | SV = 3 | | |
| LNTLDQIADIVTSVFSSPV | | P4R3C_HUMAN | Protein PPP4R3C OS = Homo | Y | |
| (SEQ ID 153) | | | sapiens OX = 9606 GN = PPP4R3C | | |
| | | | PE = 2 SV = 3 | | |
| RELSVRDVQATRVGLKD | | CTF18_HUMAN | Chromosome transmission | Y | |
| QRR (SEQ ID 154) | | | fidelity protein 18 homolog | | |
| | | | OS = Homo sapiens OX = 9606 | | |
| | | | GN = CHTF18 PE = 1 SV = 1 | | |
| APATGGVKKPHR (SEQ | | H31T_HUMAN | Histone H3.1t OS = Homo sapiens | Y | |
| ID 155) | | | OX = 9606 GN = HIST3H3 PE = 1 | | |
| | | | SV = 3 | | |
| HPHTGEFTYKSSCTFN | | LYAM2_RABIT | E-selectin OS = Oryctolagus | Y | |
| (SEQ ID 156) | | | cuniculus OX = 9986 GN = SELE | | |
| | | | PE = 2 SV = 1 | | |
| KDISVLKGTL (SEQ ID | | SIA8B_HUMAN | Alpha-2,8-sialyltransferase 8B | Y | |
| 157) | | | OS = Homo sapiens OX = 9606 | | |
| | | | GN = ST8SIA2 PE = 2 SV = 1 | | |
| KNGGFFLRIHPDGRVDG | | FGF2_MOUSE | Fibroblast growth factor 2 | Y | |
| VREKSDPHVKLQ (SEQ | | | OS = Mus musculus OX = 10090 | | |
| ID 158) | | | GN = Fgf2 PE = 1 SV = 1 | | |
| KSTREAWTDGRQSTESE | | GAB1_MESAU | GRB2-associated-binding protein | Y | |
| TPTKSV (SEQ ID 159) | | | 1 OS = Mesocricetus auratus | | |
| | | | OX = 10036 GN = GAB1 PE = 2 SV = 1 | | |
| QPGKTPKKAQNKSKPNQ | | TAPT1_XENLA | Transmembrane anterior | Y | |
| GHPPDET | | | posterior transformation protein | | |
| (SEQ ID 160) | | | 1 homolog OS = Xenopus laevis | | |
| | | | OX = 8355 GN = tapt1 PE = 2 SV = 1 | | |
| KDATGTLVGNVVHSRIIN | | NXPH2_BOVIN | Neurexophilin-2 OS = Bos taurus | Y | |
| PLRLFV (SEQ ID 161) | | | OX = 9913 GN = NXPH2 PE = 1 SV = 1 | | |
| PATTPAQPQQSQSQT | | UBAP2_HUMAN | Ubiquitin-associated protein 2 | Y | |
| (SEQ ID 162) | | | OS = Homo sapiens OX = 9606 | | |
| | | | GN = UBAP2 PE = 1 SV = 1 | | |
| LSNFIPDDEATLMKNGR | | SCNM1_DANRE | Sodium channel modifier 1 | Y | |
| YSCLVCSHRPVFDT (SEQ | | | OS = Danio rerio OX = 7955 | | |
| ID 163) | | | GN = scnm1 PE = 2 SV = 1 | | |
| ILCVRQICAVILESPPKGA | | PCBP4_BOVIN | Poly(rC)-binding protein 4 | Y | |
| TIPY (SEQ ID 164) | | | OS = Bos taurus OX = 9913 | | |
| | | | GN = PCBP4 PE = 2 SV = 1 | | |
| QHGRILDILLSTCSL | | STKL1_MOUSE | Serine/threonine kinase-like | Y | |
| (SEQ ID 165) | | | domain-containing protein | | |
| | | | STKLD1 OS = Mus musculus | | |
| | | | OX = 10090 GN = Stkld1 PE = 2 | | |
| | | | SV = 3 | | |
| QKKLIAMLENGLGLPPG | | LMOD3_HUMAN | Leiomodin-3 OS = Homo sapiens | Y | |
| MW (SEQ ID 166) | | | OX = 9606 GN = LMOD3 PE = 1 SV = 1 | | |
| INSSSSVVHKKDH (SEQ | | TAOK3_HUMAN | Serine/threonine-protein kinase | Y | |
| ID 167) | | | TAO3 OS = Homo sapiens OX = 9606 | | |
| | | | GN = TAOK3 PE = 1 SV = 2 | | |
| PQNRKEANPTPLTPGAS | | YJU2_HUMAN | YJU2 splicing factor homolog | Y | |
| SLS (SEQ ID 168) | | | OS = Homo sapiens OX = 9606 | | |
| | | | GN = YJU2 PE = 1 SV = 1 | | |
| KMVDIQVSAKKMLD | | SPRE_XENTR | Sepiapterin reductase | Y | |
| (SEQ ID 169) | | | OS = Xenopus tropicalis OX = 8364 | | |
| | | | GN = spr PE = 2 SV = 1 | | |

Mac Ret

| | | | | | |
|---|---|---|---|---|---|
| ELGVGFATR (SEQ ID | 1 | FABP4_PYGPA | Fatty acid-binding protein, | Y | [23-31] |
| 170) | | | adipocyte OS = Pygoscelis papua | | |
| | | | OX = 30457 GN = FABP4 PE = 1 SV = 1 | | |
| LGEEFDETTADDR (SEQ | 1 | FABP4_PYGPA | Fatty acid-binding protein, | Y | [67-79] |
| ID 171) | | | adipocyte OS = Pygoscelis papua | | |
| | | | OX = 30457 GN = FABP4 PE = 1 SV = 1 | | |
| AWGPGLEGGVVGK | 2 | FLNA_HUMAN | Filamin-A OS = Homo sapiens | Y | [581-593] |
| (SEQ ID 172) | | | OX = 9606 GN = FLNA PE = 1 SV = 4 | | |
| TFEPAEFIIDTR (SEQ | 2 | FLNA_HUMAN | Filamin-A OS = Homo sapiens | Y | [2062- |
| ID 173) | | | OX = 9606 GN = FLNA PE = 1 SV = 4 | | 2073] |
| VEWTQQER (SEQ ID | | HBB_THUTH | Hemoglobin subunit beta | Y | |
| 174) | | | OS = Thunnus thynnus OX = 8237 | | |
| | | | GN = hbb PE = 1 SV = 1 | | |
| ALTDAETKAFLAD (SEQ | | PRVB2_SALSA | Parvalbumin beta 2 OS = Salmo | Y | |
| ID 175) | | | salar OX = 8030 PE = 1 SV = 3 | | |
| TADLVSFTLIVGI | | GDPD5_HUMAN | Glycerophosphodiester | Y | |
| (SEQ ID 176) | | | phosphodiesterase domain- | | |
| | | | containing protein 5 OS = Homo | | |
| | | | sapiens OX = 9606 GN = GDPD5 | | |
| | | | PE = 1 SV = 2 | | |
| VVLAYEPVWAIGTGK | | TPISA_DANRE | Triosephosphate isomerase A | Y | |
| (SEQ ID 177) | | | OS = Danio rerio OX = 7955 | | |
| | | | GN = tpila PE = 2 SV = 1 | | |
| VLTWIGAQPV (SEQ ID | | CYB_PSECU | Cytochrome b OS = Pseudochirops | Y | |
| 178) | | | cupreus OX = 37702 GN = MT-CYB | | |
| | | | PE = 3 SV = 1 | | |
| SDLADLRSC (SEQ ID | | UBP34_HU | Ubiquitin carboxyl-terminal | Y | |
| 179) | | MAN | hydrolase 34 OS = Homo sapiens | | |
| | | | OX = 9606 GN = USP34 PE = 1 SV = 2 | | |
| KADLLADHTEGIIKSILQG | | MELT_MOUSE | Ventricular zone-expressed PH | Y | |
| NAMLLRVLPAVYE (SEQ | | | domain-containing protein 1 | | |
| ID 180) | | | OS = Mus musculus OX = 10090 | | |
| | | | GN = Veph1 PE = 2 SV = 2 | | |
| ARRATGNLSASC (SEQ | | RUFY1_HUMAN | RUN and FYVE domain-containing | Y | |
| ID 181) | | | protein 1 OS = Homo sapiens | | |
| | | | OX = 9606 GN = RUFY1 PE = 1 SV = 2 | | |
| KEPPNGNAFPP (SEQ ID | | LDB1_CHICK | LIM domain-binding protein 1 | Y | |
| 182) | | | OS = Gallus gallus OX = 9031 | | |
| | | | GN = LDB1 PE = 2 SV = 1 | | |
| WCSRGCQ | | PEPC_CAVPO | Gastricsin OS = Cavia porcellus | Y | |
| (SEQ ID 183) | | | OX = 10141 GN = PGC PE = 2 SV = 1 | | |
| FPFLTPEQKKELSDIAHK | | ALDOA_THUAL | Fructose-bisphosphate aldolase A | Y | |
| (SEQ ID 184) | | | (Fragment) OS = Thunnus | | |
| | | | albacares OX = 8236 GN = ALDOA | | |
| | | | PE = 1 SV = 1 | | |
| ATTVPDSNSVP (SEQ ID | | NCAM1_MOUSE | Neural cell adhesion molecule 1 | Y | |
| 185) | | | OS = Mus musculus OX = 10090 | | |
| | | | GN = Ncam1 PE = 1 SV = 3 | | |
| IVAALTMV (SEQ | | CYB_CHRAS | Cytochrome b OS = Chrysochloris | Y | |
| ID 186) | | | asiatica OX = 185453 GN = MT-CYB | | |
| | | | PE = 3 SV = 1 | | |
| KWRYKLTLFAATF (SEQ | | KCJ15_CAVPO | ATP-sensitive inward rectifier | Y | |
| ID 187) | | | potassium channel 15 OS = Cavia | | |
| | | | porcellus OX = 10141 GN = KCNJ15 | | |
| | | | PE = 3 SV = 1 | | |
| ACSLLGHV (SEQ | | PX11B_PONAB | Peroxisomal membrane protein | Y | |
| ID 188) | | | 11B OS = Pongo abelii OX = 9601 | | |
| | | | GN = PEX11B PE = 2 SV = 1 | | |

¹Amino acid nomenclature: A, ala, alanine; C, cys, cysteine; D, asp, aspartic acid; E, glu, glutamic acid; F, phe, phenylalanine; G, gly, glycine; H, his, histidine; I, lle, isoleucine; K, lys, lysine; L, leu, leucine; M, met, methionine; N, asn, asparagine; P, pro, proline; Q, gln, glutamine; R, arg, arginine; S, ser, serine; T, thr, threonine; V, val, valine; W, trp, tryptophan; Y, tyr; tyrosine; X, undetermined amino acid.
²Hit number of the assigned sequence using MASCOT search engine
³Accession numbers of protein of origin according to NCBI non-redundant database

Table 6: Identified Peptides in Mackerel and Blue Whiting 10 KDa Permeate Fractions and in Mackerel Retentate

### Example 2: Skin and Bone by-Products from Blue Whiting

Materials and Methods

Raw Material

Blue whiting by-products were kindly supplied by Bard Iascaigh Mhara (BIM) in the form of 4 kg frozen blocks. The blocks consisted of the skin, meat and bones of Blue Whiting caught in Irish waters in March 2017. 500 g was used for experiments. The material was thawed at room temperature and then cut into 1 cm pieces prior to use.

Skin and Bone Pre-Treatments

Four different pre-treatments were applied to blue-whiting by-products independently. Gelatine extraction and gelatine and gelatine hydrolysate freeze-drying steps remained the same throughout the study. The four standard operating procedures (SOPs) for pre-treatment are presented in Table 7.

TABLE 7
Pre-treatment procedures used for gelatine extraction from Blue Whiting skin and bones

| SOP | Pre-treatment | Adapted from |
|---|---|---|
| 1 | The skin and bone material was washed once using tap water (6:1 v/w | (10) |
| | to material) and pre-treated three times in consecutive order as | |
| | follows with 0.2% (w/v) NaOH (6:1 v/w skin and bone) for 40 min | |
| | followed by washing with water, then with 0.2% (v/v) sulphuric acid | |
| | (6:1 v/w to skin and bone) for 40 min followed by washing with water, | |
| | and finally using 0.05M acetic acid (6:1 v/w to skin and bone) for 40 | |
| | min followed by washing with water. | |
| 2 | The skin and bone material was washed once using tap water (6:1 v/w | (11) |
| | to material) and pre-treated three times with 0.1M NaOH (6:1 v/w to | |
| | material) for 30 min. After washing with water, it was treated with | |
| | 0.25M HCl (6:1 v/w to material) for 2 h, and then with 0.05M acetic acid | |
| | (6:1 v/w to material) for 1 h, followed by washing with water. | |
| 3 | The skin and bone material was washed once using tap water (6:1 v/w | (12) |
| | to material) and hydrolysed with alcalase enzyme (Sigma Aldrich, | |
| | Ireland; 2.5 U/ml) in 0.1M K-phosphate buffer (pH = 8; 3:1 v/w buffer to | |
| | material + 0.1% (w/w to material) enzyme) for 1.5 h at 40° C. After | |
| | heating the mixture to boiling for 5 min to inactivate the enzyme and | |
| | washing with water, the material was then treated with 0.25M HCl (6:1 | |
| | v/w to material) for 2 h and washed with water. | |
| 4 | The skin and bone material was washed once using tap water (6:1 v/w | (13) |
| | to material) and pre-treated three times with 0.1M NaOH (6:1 v/w to | |
| | material) for 30 min. After washing with water, it was treated with 1M | |
| | citric acid (6:1 v/w to material) for 2 h and finally washed with water. | |

Following the pre-treatment steps, by-product skin and bone material was washed with water until neutral (pH 6-7) and gelatine was extracted overnight using water (3:1 v/w to material) at 45° C. in a MaxQ 8000 laboratory shaker (Thermo Fisher Scientific, MA USA) at 200 rpm speed. Solids were removed by filtration through Whatman No 4 filter paper using a Büchi funnel. Resultant clear gelatine extracts were subsequently freeze-dried (FD80GP, Cuddon Freeze Dry, New Zealand) using the following program: Initial temperature −20° C.; 5 steps; run time 90 h; end temperature 0° C. Dried material was placed in sterile, sealed 250 ml containers and stored at −80° C. until analyses.

Characterisation of the Physical and Chemical Properties of Extracted Gelatines

Proximate Analysis

The yield of gelatine extracted was calculated after freeze-drying and was expressed as a percentage of the total by-product raw material used. The total protein content of the gelatine samples was determined using the Dumas combustion method using a LECO FP328 Protein analyser (LECO Corp, MI USA), according to the Association of Official Analytical Chemists (AOAC) method 992.15 (14). A conversion factor of 6.25 was used to convert total nitrogen to protein.

The ash content of the 4 different gelatine extracts was determined gravimetrically, as previously described (15).

Water Activity

Water activity (a_{w}) values for all samples were determined using an AquaLab Lite meter (Decagon Devices Inc., Germany). Approximately 0.25 g of finely milled sample was placed in the water activity meter and a_{w} and the temperature was recorded (16).

PH Value

pH values of 1% gelatine solutions (w/v) were determined at room temperature using a pH213 pH-meter (Hanna Instruments, TX USA), and buffers pH 4 and pH 7 (Sigma Aldrich, Ireland) were used for calibration in accordance with the method described by GME (17).

Lowest Gelling Concentration

The lowest gelling concentrations (LGC) of the Blue-whiting derived gelatines were determined using the method of Ogunwolu et al. (18), and results were expressed as the concentration (w/v) of gelatine solution which could form a gel at 4° C. Briefly, 2 ml of a range of concentrations of each gelatine solution (1, 2, 5, 8 and 10%; w/v) was measured in a 15 ml Falcon tube and placed in the laboratory refrigerator set to 4° C. for 2 hours. The concentration at which the solution would stop flowing in the tube was recorded as the LGC.

Colour Measurement

Colour measurements were carried out using a Minolta Lab colorimeter (CR-400/410, Konica, Minolta, Ireland) in accordance with previously described methods (19). White and black standard tiles were used to calibrate the instrument prior to measurements. Readings were reported in the CIE L*, a*, and b* system, as L* (lightness), a* (redness/greenness), and b* (yellowness/blueness). The chroma (C*) values were calculated using the following equation (20):

C*=\sqrt{a²×b²}

Sodium Dodecyl Sulphate-Polyacrylamide Gel Electrophoresis (SDS-PAGE)

Extracted gelatine samples were prepared for sodium dodecyl sulphate polyacrylamide electrophoresis (SDS-PAGE) analysis according to a previously described method (7). SDS-PAGE was carried out using the discontinuous Tris-tricine buffer system (Sigma Aldrich, Ireland). Briefly, gelatine extracts (1%, w/v) were diluted with sample buffer containing 0.2 M Tris-HCl, pH 6.8 containing 2% (w/v) SDS, 40% (v/v) glycerol, 0.04% (w/v) Coomasie Blue G250 and 2% (v/v) 2-mercaptoethanol at the ratio of 1:1 (v/v) and heated to 95° C. for 5 min. The gels used for electrophoresis were the Bio-Rad Mini-Protean Tris-tricine precast gels (4-20%). Gelatine samples were run in a Mini-Protean® Tetra Cell (Bio-Rad Laboratories, Watford, United Kingdom) at a constant voltage of 100 V. Gels were then fixated in 40% methanol, 10% acetic acid (v/v, in water) and stained with Biosafe Coomassie G250 Stain.

The load volume was 20 μL in all lines. A Bio-Rad Precision Plus Protein™ unstained protein molecular weight standard (Bio-Rad Laboratories, Watford, United Kingdom), MW range (10-250 KDa) and a LW range standard (4-250 kDa) was used to identify the protein fractions. Band detection and molecular weight calculation was done using GelAnalyzer 2010a software (http://www.gelanalyzer.com).

Determination of Techno-Functional Properties

Water and Oil Holding Capacities

The water holding capacity (WHC) and oil holding capacity (OHC) of extracted gelatines were measured using the modified methods described by Neto et al. (21). In brief, 100 mg of each gelatine sample was mixed with 1 ml of distilled water or oil (olive and sunflower oil) using a vortex mixer (VV3, VWR International Ltd., Ireland) for 30 seconds. The suspension was then centrifuged at 2200×g for 30 min (Eppendorf MiniSpin, Eppendorf UK Limited, United Kingdom) at 19° C. The supernatant was then decanted by draining the tubes at 45° angle for 10 min (20 min for oil). Water/oil holding capacity was calculated by dividing the weight of water/oil absorbed by the weight of the protein sample and expressed as grams of water or sunflower oil held by 1 g of protein.

Solubility

Solubility in water (S) was determined using the modified method of Ogunwolu et al. (18). 100 mg of each gelatine sample was dispersed in 10 ml de-ionised water. Using a pH-meter (Mettler Toledo, Barcelona, Spain), the pH of the dispersions was adjusted to 2, 4, 6, 8 and 10 with 0.1 N HCl and 0.1 N NaOH. The mixture was vortexed at room temperature (19-20° C.) for 5 min, and subsequently centrifuged at 7500×g for 15 min (Lynx 6000, Thermo Fisher Scientific, MA USA). The protein content in the supernatant was determined using the Pierce™ BCA (bicinchoninic acid) Protein Assay Kit (Thermo Fischer Scientific, MA USA). Gelatine solubility at different pH conditions was then calculated using the following equation:

S (%)=(protein content in the supernatant/protein content in full dispersion)×100.

Foaming Capacity

The foaming capacity (FC) of the gelatine samples was determined by the modified method described by Bencini (22). Gelatine was suspended in deionized water to make 1% concentration (w/v) and the pH was adjusted to 2, 4, 6, 8 and 10 with 0.1 N HCl and 0.1 N NaOH. The suspensions were homogenized using a T 25 digital ULTRA-TURRAX® homogenizer (IKA®, Germany) (10,000 rpm; 1 min) and the volume of produced foam was measured in a graduated 50 ml Falcon tube. FC was calculated as the volume of foam using the formula:

FC (%)=(V_{f}−V₀)/V₀×100

where; V0 is the initial volume of protein solution before homogenization and Vf is the volume of foam produced after homogenization. The foaming stability (FS) was calculated using the same formula, with Vf measured after 15, 30 and 60 min.

Emulsifying Activity and Emulsifying Stability

The emulsifying activity (EA) of the protein extracts was determined using a modified method of Garcia-Vaquero et al. (20). Gelatine was suspended in deionized water to make 1% concentration (w/v) and the pH was adjusted to 2, 4, 6, 8 and 10 with 0.1 N HCl and 0.1 N NaOH. The solution was homogenized for 30 s at 14,000 rpm using a T 25 digital ULTRA-TURRAX® homogenizer (IKA®, Germany). The emulsion was created by addition of sunflower oil to aqueous phase (oil:gelatine solution=3:2) in 50 ml Falcon tubes. The oil was added in two steps: half of the volume was first added, and the mixture was homogenized 30 s at 14000 rpm, after which the rest of the oil was added and homogenization was repeated for 90 s at the same speed. The tubes containing emulsion were then centrifuged at 1100×g for 5 min and the volume of the emulsion layer was recorded. Emulsifying activity was calculated by the formula:

EA (%)=(Ve/Vt)×100

Where; Ve was the volume of the emulsion layer after centrifuging and Vt was the complete volume inside the tube. Emulsion stability (ES) was determined by heating the previously prepared emulsions at 85° C. for 15 min, cooling at room temperature for 10 min and centrifuging again 1100×g for 5 min. The ES was calculated and expressed as the % of EA after heating and centrifuging by the formula:

ES (%)=(V_{emulsion after heating}/V_{original emulsion})×100

Microsoft Excel was used for all calculations and graph construction in this study, except in the case of SDS-Page gel analysis, where the GelAnalyzer 2010a software was used for molecular weight estimation. Purified gelatine of bovine (BOV) and porcine (POR) origin (Sigma Aldrich, Ireland) were used for comparison of properties.

Enzyme Hydrolysis

Based on the previously determined chemical, physical and functional properties, gelatine extracts generated using SOP1 and SOP2 were selected for further hydrolysis using the commercial enzymes alcalase and papain. The hydrolysis procedures were undertaken as follows, using a modified procedure described by Lafarga and Hayes (23):

Gelatine samples (2.5 g) were dissolved in deionized water (250 ml) to obtain 1% (w/v) gelatine solutions, which were hydrolysed independently with either Alcalase® (EC number 3.4.21.62, activity ≥5 U/g, Sigma Aldrich, Ireland) and papain (EC number 3.4.22.2, activity ≥3 U/mg, Sigma Aldrich, Ireland). The pH values and temperature of each gelatine solution was set before addition of the enzymes as follows: 60° C. and pH 9.5 for Alcalase, 65° C. and pH 6.5 for papain. Solutions were kept in an incubated laboratory shaker (at optimum temperatures, as specified above) during hydrolysis and agitated at 200 rpm. The alcalase was added in a substrate-to enzyme ratio of 100:1 (w/w) and papain was added in 10:1 (w/w) ratio. After incubation, the solutions were heated at 95° C. for 10 min in a water bath to deactivate the enzymes. The pH of solutions was maintained at the optimum value for each enzyme using 0.1 M or 1 M NaOH. After incubation, the degree of hydrolysis (DH) was calculated using the pH-stat method (24), after 1, 60, 120 and 240 min. The OH was then calculated using the following equation:

DH=B×N_{B}×1/α×1/Mₚ×1/hₜₒₜ×100%,

where B (mL) is the volume of NaOH consumed, N_{B} is the normality of the NaOH used, 1/a is the average degree of dissociation of the α-amino groups related with the pK of the amino groups at particular pH and temperatures, M_{P} (g) is the amount of protein in the reaction mixture, and hₜₒₜ (meq/g) is the sum of the millimoles of individual amino acids per gram of protein associated with the source of protein used in the experiment. Values for hₜₒₜ and 1/a were obtained from the study conducted by Adler-Nissen (24). Gelatine hydrolysates were freeze-dried as previously described and dried product was kept in sealed containers at −80° C. until further use.

Molecular Weight Cut Off Filtration

Gelatine hydrolysates (0.5 g) were diluted independently in deionized water (10 ml) to a concentration of 5% (w/v). The solutions (10 ml) were then filtered through 3 kDa membrane filters using the Amicon Ultra-15 Centrifugal Filter Unit (Sigma Aldrich, Ireland) at 4000 g for 60 min to obtain fractions with peptides less than 3 kDa in size. Filtrates and permeates were then freeze-dried and the yield of each peptide fraction (s 3 KDa) was calculated after weighing.

ACE-I Inhibition Assay

ACE-I inhibition activity of gelatine hydrolysates and 3 kDa fractions was determined using a bioassay kit in accordance with the manufacturer's instructions (ACE Kit—WST, Dojindo Laboratories, Kumamoto, Japan). In brief, 20 μL of each peptide aqueous solution at a concentration of 1 mg/mL was added to 20 μL substrate and 20 μL enzyme working solution in triplicate. Captopril was used as a positive control (0.5 mg/mL). Samples were incubated at 37° C. for 1 h. A 200 μL of indicator working solution was then added to each well, and subsequent incubation at room temperature was carried out for 10 min. Absorbance at 450 nm was read using a FLUOstarOmega microplate reader (BMG LABTECH GmbH, Offenburg, Germany). The percentage of inhibition was calculated using the following equation:

% ACE-I inhibition=100% Initial activity−Inhibitor×100/100% Initial activity

Amino Acid Composition

The total amino acid content of selected gelatine hydrolysates was determined using the method described by Hill (25). Samples were hydrolysed in 6 M HCl at 110° C. for 23 h. The hydrolysed solutions were diluted with 0.2 M sodium citrate buffer (pH 2.2) to give approximately 250 nmol of each amino acid residue. The sample was then diluted 1:1 with the internal standard, norleucine, to give a final concentration of 125 nmol/mL. Amino acids were quantified using a Jeol JLC-500/V amino acid analyser (Jeol Ltd., Garden city, Herts, UK) fitted with a Jeol Na⁺ high-performance cation exchange column.

MS Analysis

Gelatine papain hydrolysates generated from gelatines isolated with SOP1 and SOP2 were selected for MS analysis based on results from techno-functional and bioactivity assays. Samples were cleaned prior to MS analysis using the TiO₂ clean-up kit (Pierce, USA). Samples were re-suspended in 50 uL of 0.1% TFA in 2% ACN. 5 μl of every sample was loaded onto a trap column (NanoLC Column, 3μ C18-CL, 350 mm×0.5 mm; Eksigent) and desalted with 0.1% TFA at 3 μl/min during 5 min.

The peptides were then loaded onto an analytical column (LC Column, 3μ C18-CL, 75 um×12 cm, Nikkyo) equilibrated in 5% acetonitrile 0.1% FA (formic acid). Elution was carried out with a linear gradient of 5 to 35% B in A for 45 min. (A: 0.1% FA; B: ACN, 0.1% FA) at a flow rate of 300 nl/min. Peptides were analysed in a mass spectrometer nanoESI qQTOF (5600 TripleTOF, ABSCIEX). Sample was ionized applying 2.8 kV to the spray emitter. Analysis was carried out in a data-dependent mode. Survey MS1 scans were acquired from 350-1250 m/z for 250 ms. The quadrupole resolution was set to ‘UNIT’ for MS2 experiments, which were acquired 100-1500 m/z for 50 ms in ‘high sensitivity’ mode. Following switch criteria were used: charge: 1+ to 5+; minimum intensity; 70 counts per second (cps). Up to 25 ions were selected for fragmentation after each survey scan. Dynamic exclusion was set to 15 s.

Data Analysis Procedure

ProteinPilot (version 5.0) software from Sciex was used in the data analysis. ProteinPilot default parameters were used to generate peak list directly from 5600 TripleTof wiff files. The Paragon algorithm (26) of ProteinPilot v 5.0 was used to search the NCBI_collagen (162534 searched proteins) database with the following parameters: no enzyme specificity, no taxonomy restriction, and the search effort set to through. The protein grouping was done by Pro group algorithm.

Results

Proximate Analysis

The choice of pre-treatment procedure had a significant impact on the yield and proximate composition of the extracted gelatines, as can be seen in Table 8. The yield of dry gelatine recovered was 4.55±0.53% for SOP1, 4.73±0.41% for SOP2, 2.22±0.51% in the case of SOP3, while SOP4 resulted in a yield of 5.02±0.89% gelatine. Although yields are low relative to previous work [(27), (3), (28)], reports on gelatine extraction yields vary greatly throughout available literature [(29), (27), (30), (31)]. The crude protein content of the samples was 86.4±2.49% for SOP1, 85.6±2.41 in SOP2, 67.6±0.93 in SOP3 and 67.4±5.03% in SOP4 gelatine sample, while it was 91.7±0.27% and 94.8±0.29% in the case of control bovine and porcine gelatines, respectively. The SOP1 and SOP2 samples had similar ash content (5.06±0.15 and 5.95±0.19%, respectively), while SOP3 sample had an unusually high (23.3±2.86%) content of ash, and SOP4 sample contained 11.6±0.09% ash. These determined values were significantly higher than in bovine and porcine control samples, which both contained less than 1% ash (0.68±0.12 and 0.24±0.10%, respectively).

TABLE 8
physical properties and chemical composition of the gelatine samples

| | | | LGC¹ | | | |
|---|---|---|---|---|---|---|
| | | | (%, | | | |
| | Yield (%, | | w/v), at | Protein | Ash (%, | |
| | w/w) | pH | 4° C. | (%, w/w) | w/w) | Aw² |
| SOP1 | 4.55 ± 0.53 | 7.05 ± 0.02 | 2 | 86.4 ± 2.49 | 5.06 ± 0.15 | 0.26 ± 0.01 |
| SOP2 | 4.73 ± 0.41 | 6.59 ± 0.02 | 2 | 85.6 ± 2.41 | 5.95 ± 0.19 | 0.44 ± 0.00 |
| SOP3 | 2.22 ± 0.51 | 4.61 ± 0.02 | 8 | 67.6 ± 0.93 | 23.3 ± 2.86 | 0.52 ± 0.01 |
| SOP4 | 5.02 ± 0.89 | 5.03 ± 0.02 | 2 | 67.4 ± 5.03 | 11.6 ± 0.09 | 0.39 ± 0.01 |
| BOV³ | / | 5.77 ± 0.01 | 1 | 91.7 ± 0.27 | 0.68 ± 0.12 | 0.42 ± 0.01 |
| POR⁴ | / | 7.57 ± 0.01 | 1 | 94.8 ± 0.29 | 0.24 ± 0.10 | 0.36 ± 0.01 |

¹LGC—lowest gelling concentration;
²Aw—water activity
³BOV—bovine gelatine;
⁴POR—orcine gelatine

Water Activity, pH Value and LGC

The pH values of 1% (v/w) aqueous gelatine solutions are shown in Table 8. The values for SOP1 and SOP2 samples (pH 7.05±0.02 and pH 6.59±0.02, respectively) were similar to the pH values of the porcine control sample (7.57±0.01); while pH of SOP3 and SOP4 gelatines (4.61±0.02 and 5.03±0.02, respectively) was closer to value determined for bovine gelatine (5.77±0.01). The a_{w} values of the examined samples were 0.26±0.01, 0.44±0.00, 0.52±0.01 and 0.39±0.01 for SOP1, SOP2, SOP3 and SOP4 extracted gelatines after drying, respectively. Bovine and porcine gelatines had comparable water activity (0.42±0.01 and 0.36±0.01, respectively). The lowest gelling concentration (LGC) values of the samples (Table 8) show that SOP1, SOP2 and SOP4 solutions were able to form gels at 4° C. at 2% concentration (w/v), while SOP3 gelatine did not show gelling up until 8% concentration. Both control mammalian gelatines formed gels at 1% concentrations.

Colour Measurement

The colour parameters of the examined gelatines are presented in Table 9. It can be seen that values of L (lightness) was 95.3±0.73 in the case of SOP1 gelatine, 95.3±0.73 for SOP2, 82.7±3.14 for SOP3 and 9.4±1.93 for SOP4 sample. The b parameter (yellowness/blueness) showed a wide range of determined values, between 2.58±0.85 and 29.5±5.69 (SOP4 and SOP3 samples, respectively); similarly to calculated C value (chroma) of the samples, which was in the range between 2.62 (SOP4) and 29.62 (SOP3). The a parameter (greenness/redness) of all measured samples had negative value, indicating a slight colour shift toward greenness.

TABLE 9
colour parameters of the gelatine samples

| | L | a | b | C |
|---|---|---|---|---|
| SOP1 | 86.2 ± 2.40 | −0.09 ± 0.08 | 3.95 ± 0.67 | 3.95 |
| SOP2 | 95.3 ± 0.73 | −0.35 ± 0.09 | 3.02 ± 0.39 | 3.04 |
| SOP3 | 82.7 ± 3.14 | −2.85 ± 1.44 | 29.5 ± 5.69 | 29.62 |
| SOP4 | 94.4 ± 1.93 | −0.49 ± 0.16 | 2.58 ± 0.85 | 2.62 |
| BOV¹ | 85.9 ± 0.96 | −0.99 ± 0.22 | 21.4 ± 1.15 | 21.41 |
| POR² | 91.4 ± 0.12 | −0.91 ± 0.02 | 12.9 ± 0.22 | 12.98 |

¹BOV—bovine gelatine;
²POR—porcine gelatine

Sodium Dodecyl Sulphate-Polyacrylamide Gel Electrophoresis (SOS-PAGE)

The distribution of molecular weights (MV) in the gelatine samples is presented in FIG. 8. After identification of bands and calculations of MW using the GelAnalyzer2010a software, the following bands were identified in the examined samples:
SOP1: 392 (γ-chain), 232 (β-chain), 134 (α1-chain), 112 (α2-chain), 70, 28, 26, 25
SOP2: 348 (γ-chain), 215 (β-chain), 117 (α2-chain)
SOP3: 215 (β-chain), 117 (α2-chain), 74, 54, 36, 31, 28, 26
SOP4: 377 (γ-chain), 215 (β-chain), 114 (α2-chain)
BOV: 433 (γ-chain), 232 (β-chain), 137 (α1-chain), 39, 25
POR: 223 (β-chain), 137 (α1-chain), 53, 33, 26, 25

Water and Oil Holding Capacity

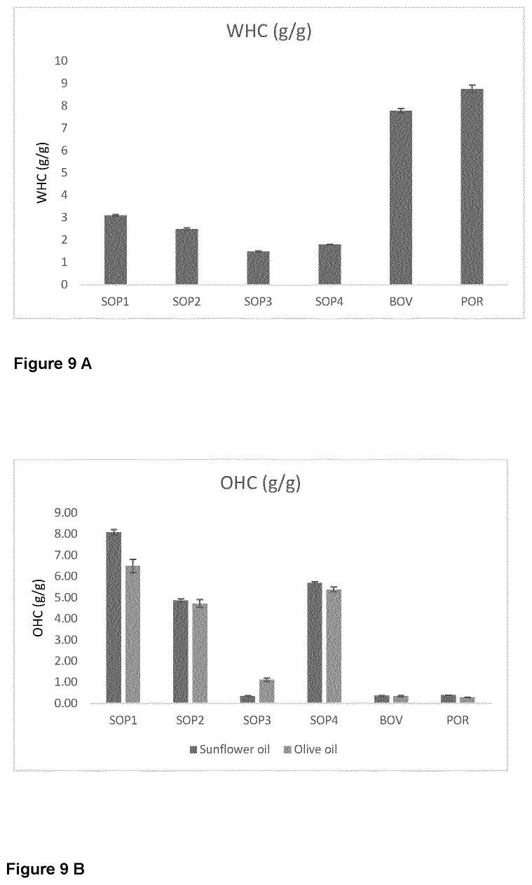

The water holding (WHC) and oil holding capacity (OHC) of the fish gelatine samples are presented in FIG. 9. WHC values for the tested fish gelatines were 3.10±0.04 g/g for gelatine extracted using SOP1, 2.48±0.06 g/g for gelatine extracted using SOP2, 1.48±0.03 g/g for gelatine extracted using SOP3 and 1.79±0.02 g/g for SOP4 gelatine. The control bovine and porcine gelatines had significantly higher WHC values (7.78±0.09 and 8.75±0.17 g/g, respectively). However, OHC values for both sunflower and olive oil were significantly higher for SOP1 (8.08±0.13 g/g and 6.49±0.32 g/g for sunflower and olive oil, respectively) SOP2 (4.86±0.07 g/g and 4.72±0.19 g/g for sunflower and olive oil, respectively) and SOP4 gelatine (5.69±0.05 g/g and 5.38±0.11 g/g for sunflower and olive oil, respectively) samples than values determined for bovine (0.36±0.02 g/g and 0.34±0.03 g/g for sunflower and olive oil, respectively) and porcine (0.39±0.01 g/g and 0.28±0.01 g/g for sunflower and olive oil, respectively) gelatines. OHC values for SOP3 gelatine (0.34±0.02 g/g and 1.12±0.09 g/g for sunflower and olive oil, respectively) were similar to values determined for control gelatines, and significantly lower than the rest of fish gelatine samples.

Solubility, Foaming Capacity and Foaming Stability

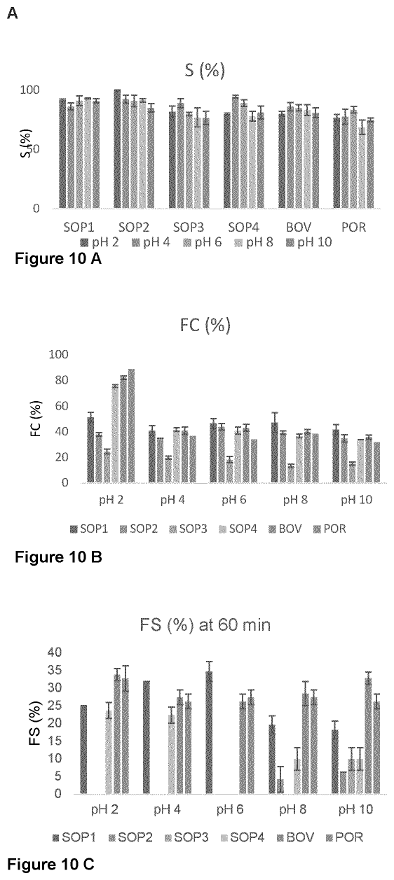

Solubility (S) of 1% (w/v) gelatine solutions at pH ranges between 2 and 10 is presented in FIG. 10. The fish gelatines displayed solubility ranging from 76.4±5.63% (SOP3 sample at pH 10) to 99.6±0.52% (SOP2 sample at pH 2). The solubility of control gelatine samples was determined to range from 68.7±5.93% (porcine gelatine sample at pH 8) to 86.0±3.57% (bovine gelatine sample at pH 4).

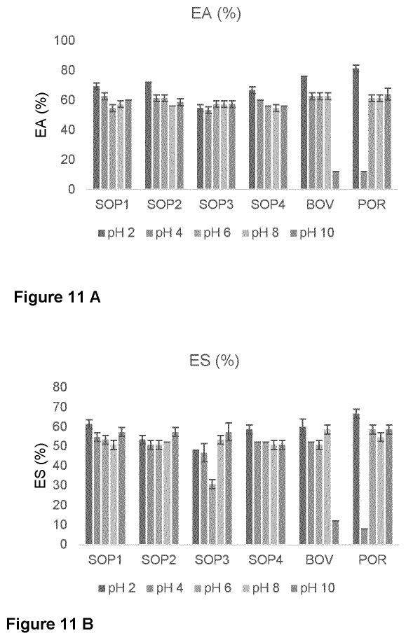

The foaming capacity (FC) and foaming stability (FS) of the 1% (w/v) gelatine sample solutions at different pH values (2, 4, 6, 8 and 10) are presented in FIG. 11. FC values were in the range between 13.4±2.83% (SOP3 at pH 8) and 75.6±7.70% (SOP4 at pH 2) in the case of fish gelatine samples. After 60 minutes, the ability to maintain foam decreased significantly and several samples had no foam layer (SOP2 at pH 2, 4 and 6; SOP3 at pH 2, 4, 6 and 8 an SOP4 at pH6), as can be seen from FS values in FIG. 10. The control bovine and porcine gelatine samples did not completely lose the FC at any of the tested pH levels, although the determined FS values show a significant decrease foaming activity in general.

Emulsifying Activity and Emulsifying Stability

The emulsifying activity (EA) and emulsifying stability (ES) of 1% (w/v) fish gelatine solutions at pH range from 2 to 10 are presented in FIG. 11. EA values of fish gelatine samples were in the range between 53.3±2.31% (SOP3 sample at pH 4) and 72.00±0.00% (SOP2 sample at pH 2), while the control samples showed a wider range of determined EA values from 12.00±0.00% (bovine gelatine sample at pH 10) to 81.3±2.31% (porcine gelatine sample at pH 2). ES values showed a slight decrease in emulsifying properties, ranging from 30.7±2.31% to 61.3±2.31% (SOP3 at pH 6 and SOP1 sample at pH 2, respectively) and between 8.00±0.00% and 66.7±2.31% in the case of control gelatine samples (porcine sample at pH4 and porcine sample at pH 2, respectively).

Enzyme Hydrolysis

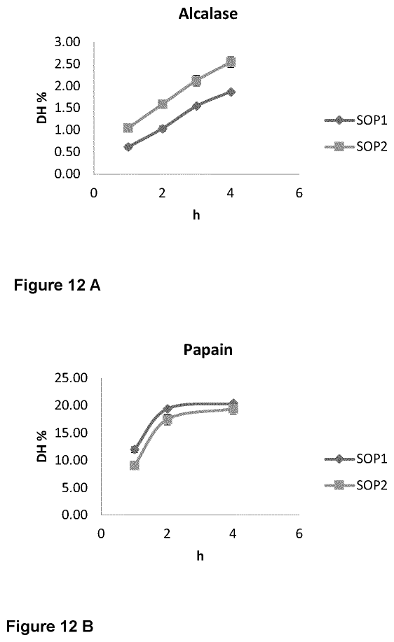

The degree of hydrolysis (DH) of SOP1 and SOP2 gelatine sample solutions is presented in FIG. 12. After 4 h of hydrolysis, when alcalase was used, the DH reached 1.87±0.02% and 2.54±0.12% (SOP1 and SOP2 samples, respectively) and with papain these values were 20.35±0.01% and 19.38±0.97% (SOP1 and SOP2 samples, respectively). Since papain in this study was used in higher ratio to sample (1:10, w/w) than alcalase (1:100, w/w) the graph of papain DH shows that, under the applied conditions, the hydrolysis was approaching a plateau at DH values close to 20%.

After molecular weight cut-off (MWCO), using 3 kDa membranes the SOP1 and SOP2 papain hydrolysates had yields of 42.1±0.54% and 43.2±1.67%, respectively, while the alcalase hydrolysates of gelatines from SOP1 and SOP2 yielded 3 kDa hydrolysate fractions of 10.9±0.89% and 9.3±0.61% respectively.

Amino Acid Composition

The total amino acid composition of SOP1 and SOP2 gelatine samples and their papain hydrolysates are presented in Table 10. The main amino acids detected included glycine (190.4 to 210.6 g/Kg), glutamic acid (78.7 to 84.6 g/Kg), alanine (72.7 to 80.5 g/Kg) and proline (76.6 to 86.7 g/Kg). The sum of the total amino acids ranged from 749.9 g/Kg (SOP2 papain hydrolysate) to 800.9 g/Kg (SOP1 papain hydrolysate), which accounted for 74.9% and 80.1% of the total sample weights, respectively.

TABLE 10
Amino acid composition of SOP1 and SOP2 gelatine samples and
their papain hydrolysate fractions ACE-I inhibition assay

| Amino acid (g/Kg): | SOP1 | SOP1 hydrol | SOP2 | SOP2 hydrol |
|---|---|---|---|---|
| Cysteic acid | 0.247 | 3.40 | 0.214 | 3.66 |
| Taurine | 5.05 | 3.04 | 2.77 | 2.11 |
| Methionine sulfone | 24.9 | 23.1 | 23.3 | 20.8 |
| Asp | 49.5 | 51.3 | 50.8 | 47.9 |
| Thr | 22.1 | 23.8 | 22.7 | 21.7 |
| Ser | 42.4 | 47.9 | 43.7 | 41.7 |
| Glu | 80.4 | 84.6 | 81.9 | 78.7 |
| Gly | 202.2 | 205.6 | 210.6 | 190.4 |
| Ala | 77.8 | 80.4 | 80.5 | 72.7 |
| Cysteine | 4.28 | 3.72 | 2.66 | 2.77 |
| Val | 16.4 | 18.3 | 17.1 | 19.8 |
| Ile | 9.99 | 8.83 | 10.1 | 11.2 |
| Leu | 18.8 | 19.7 | 20.0 | 20.7 |
| Tyr | 2.86 | 5.87 | 1.72 | 5.69 |
| Phe | 18.5 | 20.1 | 19.5 | 18.7 |
| His | 11.9 | 11.8 | 12.4 | 11.1 |
| Lys | 28.0 | 32.3 | 29.0 | 30.5 |
| NH3 | 6.99 | 5.87 | 7.47 | 6.52 |
| Arg | 68.9 | 69.7 | 73.1 | 66.7 |
| Pro | 81.8 | 81.6 | 86.7 | 76.6 |
| SUM (g/Kg) | 773.0 | 800.9 | 796.2 | 749.9 |

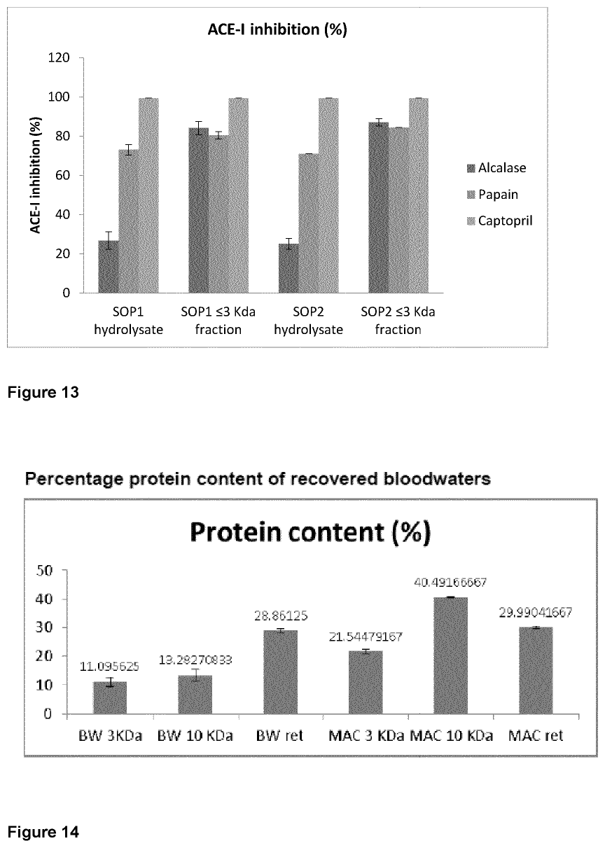

The results of ACE-1 inhibition assay are presented in FIG. 13. The alcalase hydrolysates had ACE-1 inhibition values of 26.8±4.47% and 25.1±2.72% for SOP1 and SOP2 gelatine hydrolysates, respectively when compared to Captopril (99.48% at 0.5 mg/ml). The 53 KDa peptide fractions of the corresponding alcalase hydrolysates had 84.2±3.38% and 87.1±1.83% ACE-1 inhibition when assayed at 1 mg/ml concentrations. Gelatine hydrolysates generated using papain resulted in ACE-1 inhibition values of 73.1±2.72% and 71.1±0.00% for SOP1 and SOP2 gelatine hydrolysates, respectively when compared to Captopril (99.48% at 0.5 mg/ml). The 53 KDa peptide fractions of the corresponding hydrolysates had 80.5±1.83% and 84.5±0.00% ACE-1 inhibition when assayed at 1 mg/ml concentrations.

MS Analysis

MS analysis of the 3 kDa fractions generated from gelatines isolated using SOP1 and SOP2 and hydrolysed with papain are presented in Table 11. A total of 81 peptides were identified in the papain gelatine hydrolysate made using gelatine isolated with SOP1, while 90 peptides were identified in the hydrolysate generated using papain and gelatine isolated with SOP2. All the identified peptides were identified as novel when checked against the BIOPEP database of known peptides.

TABLE 11
Identified peptides in papain hydrolysates ≤ 3 KDa peptide fractions

| | | | | | | |
|---|---|---|---|---|---|---|
| | | | Unique | | | |
| | | | in | | | Position |
| | | Con- | BIOPEP | NCBI accession | Confirmed in NCBI database | in protein |
| Peptide Sequence1 | Modifications | fidence² | database | number³ | (corresponding protein) | sequence |

SOP1 papain hydrolysate

| | | | | | | |
|---|---|---|---|---|---|---|
| GPPGPPGPPGPPG | | 99.0 | Y | XP_008299760.1 | PREDICTED: collagen alpha-1(I) | [1092-1104] |
| (SEQ ID 189) | | | | | chain-like isoform X2 [Stegastes | |
| | | | | | partitus] | |
| PAVPGPMG (SEQ ID | | 99.0 | Y | XP_008299760.1 | PREDICTED: collagen alpha-1(1) | [159-166] |
| 190) | | | | | chain-like isoform X2 [Stegastes | |
| | | | | | partitus] | |
| PAVPGPMGPM (SEQ | Oxidation | 99.0 | Y | XP_008299760.1 | PREDICTED: collagen alpha-1(I) | [159-168] |
| ID 191) | (M)@10 | | | | chain-like isoform X2 [Stegastes | |
| | | | | | partitus] | |
| GPLGPPGLPGPPG (SEQ | | 99.0 | Y | XP_020469236.1 | | |
| ID 192) | | | | | | |
| GAPGPIGPPGPPGPPG | | 99.0 | Y | XP_009984740.1 | PREDICTED: collagen alpha- | [190-205) |
| (SEQ ID 193) | | | | | 1(XXVI) chain-like [Tauraco | |
| | | | | | erythrolophus] | |
| GPIGPPGPPGPPG (SEQ | | 99.0 | Y | XP_009984740.1 | PREDICTED: collagen alpha- | [190-202] |
| ID 194) | | | | | 1(XXVI) chain-like [Tauraco | |
| | | | | | erythrolophus] | |
| GPIGPPGPPGPPGA | | 99.0 | Y | XP_009984740.1 | PREDICTED: collagen alpha- | [193-206] |
| (SEQ ID 195) | | | | | 1(XXVI) chain-like [Tauraco | |
| | | | | | erythrolophus] | |
| GPPGPPGPNGPPG | | 99.0 | Y | XP_006814188.1 | PREDICTED: collagen alpha-5(IV) | [250-262; |
| (SEQ ID 363) | | | | | chain-like [Saccoglossus | 364-375; |
| | | | | | kowalevskii] | 602-613] |
| GPPGPPGPPGPPGF | | 99.0 | Y | XP_006814188.1 | PREDICTED: collagen alpha-5(IV) | [174-187; |
| (SEQ ID 196) | | | | | chain-like [Saccoglossus | 534-547; |
| | | | | | kowalevskii] | 742-755] |
| AVGLRGSPGADGAIGP | | 99.0 | Y | XP_014216795.1 | PREDICTED: collagen alpha-1(I) | [408-424] |
| K (SEQ ID 197) | | | | | chain-like [Copidosoma | |
| | | | | | floridanum] | |
| GPLGPIGPLG (SEQ ID | | | Y | XP_014216795.1 | PREDICTED: collagen alpha-1(I) | [215-224] |
| 198) | | | | | chain-like [Copidosoma | |
| | | | | | floridanum] | |
| GLPGAPGLPGPPA (SEQ | | 99.0 | Y | XP_019617011.1 | PREDICTED: collagen alpha-2(IV) | [678-690] |
| ID 199) | | | | | chain-like [Branchiostoma | |
| | | | | | belcheri] | |
| LPGAPGLPGPPA (SEQ | | 99.0 | Y | XP_019617011.1 | PREDICTED: collagen alpha-2(IV) | [679-690] |
| ID 200) | | | | | chain-like [Branchiostoma | |
| | | | | | belcheri] | |
| GPAGIPGPIGPPGPPG | | 99.0 | Y | XP_012500099.1 | PREDICTED: collagen alpha-5(IV) | [186-201) |
| (SEQ ID 201) | | | | | chain [Propithecus coquereli] | |
| GIPGPIGPPGPPG (SEQ | | 99.0 | Y | XP_012500099.1 | PREDICTED: collagen alpha-5(IV) | [189-201] |
| ID 202) | | | | | chain [Propithecus coquereli] | |
| DCTYIPGIPGLPGSPGP | | 99.0 | Y | KGL86909.1 | Collagen alpha-1(IV) chain, | [440-456] |
| (SEQ ID 203) | | | | | partial | |
| | | | | | [Charadrius vociferus] | |
| PGLPGPMG (SEQ ID | | 99.0 | Y | XP_010950646.1 | PREDICTED: collagen alpha-1(IV) | [1183-1190] |
| 204) | | | | | chain [Camelus bactrianus] | |
| GLPGPPGLPGPAGP | | 99.0 | Y | XP_013889920.1 | PREDICTED: collagen alpha- | [726-739] |
| (SEQ ID 205) | | | | | 1(XXII) chain isoform X1 | |
| | | | | | [Austrofundulus limnaeus] | |
| GLPGPLGPPGPPGM | | 99.0 | Y | CCD75837.1 | hypothetical protein | [1040-1053] |
| (SEQ ID 206) | | | | | Smp_196840 [Schistosoma | |
| | | | | | mansoni] | |
| GLPGPLGPPGPPG (SEQ | | 99.0 | Y | CCD75837.1 | hypothetical protein | [1040-1052] |
| ID 207) | | | | | Smp_196840 [Schistosoma | |
| | | | | | mansoni] | |
| PGGSGPVGPQ (SEQ ID | | 99.0 | Y | XP_014738660.1 | PREDICTED: collagen alpha- | [872-881] |
| 208) | | | | | 1(XXIV) chain [Sturnus vulgaris] | |
| GLPGPLGPPGPGGP | | 99.0 | Y | XP_015496438.1 | PREDICTED: collagen alpha-1(VII) | [2260-2273] |
| (SEQ ID 209) | | | | | chain isoform X1 [Parus major] | |
| PSQPGSGGQGGSGGLP | Deamidated | 99.0 | Y | XP_018333494.1 | PREDICTED: collagen alpha-2(IV) | [789-807] |
| GTP (SEQ ID 210) | (Q)@3; | | | | chain-like [Agrilus planipennis] | |
| | Deamidated | | | | | |
| | (Q)@9 | | | | | |
| PGIKGLPGLPGPPGP | | 99.0 | Y | XP_004005028.1 | | |
| (SEQ ID 211) | | | | | | |
| GLPGPPGPLGPDW | | 99.0 | Y | XP_016129114.1 | PREDICTED: collagen alpha- | [519-531] |
| (SEQ ID 212) | | | | | 1(XVIII) chain-like isoform X1 | |
| | | | | | [Sinocyclocheilus grahami] | |
| GLRGPQGAPGEQ (SEQ | | 99.0 | Y | XP_018017963.1 | PREDICTED: collagen alpha-1(I) | [1116-1127] |
| ID 213) | | | | | chain-like isoform X1 [Hyalella | |
| | | | | | azteca] | |
| GPTGPMGPTGLPGPLG | | 99.0 | Y | XP_017271376.1 | collagen alpha-1(VII) chain | [2272-2287] |
| (SEQ ID 214) | | | | | isoform X2 [Kryptolebias | |
| | | | | | marmoratus] | |
| PSEAPAFPCST (SEQ ID | | 99.0 | Y | XP_008009317.1 | PREDICTED: collagen alpha-1(XX) | [126-136] |
| 215) | | | | | chain isoform X1 [Chlorocebus | |
| | | | | | sabaeus] | |
| GDPGPPGLPGLPGPSG | | 99.0 | Y | XP_020639976.1 | collagen alpha-6(IV) chain | [829-845] |
| P (SEQ ID 216) | | | | | [Pogona vitticeps] | |
| LGTLGMLGIEGI (SEQ | Oxidation | 99.0 | Y | XP_014166856.1 | PREDICTED: collagen alpha-1(VIII) | [28-39] |
| ID 217) | (M)@6 | | | | chain-like, partial [Geospiza | |
| | | | | | fortis] | |
| EKGEPGSPGDQGPTGP | | 99.0 | Y | XP_010197359.1 | PREDICTED: collagen alpha-1(XIII) | [255-270] |
| (SEQ ID 218) | | | | | chain, partial [Colius striatus] | |
| PQGPIGPPGPLGPPG | | 99.0 | Y | XP_007231079.1 | PREDICTED: splicing factor, | [890-904] |
| (SEQ ID 219) | | | | | arginine/serine-rich 15 isoform | |
| | | | | | X1 [Astyanax mexicanus] | |
| GPIGPPGPLGPPG (SEQ | | 99.0 | Y | XP_007231079.1 | PREDICTED: splicing factor, | [892-904] |
| ID 220) | | | | | arginine/serine-rich 15 isoform | |
| | | | | | X1 [Astyanax mexicanus] | |
| APGPVPMA (SEQ ID | | 99.0 | Y | XP_015475081.2 | PREDICTED: collagen triple helix | [95-102] |
| 221) | | | | | repeat-containing protein 1 | |
| | | | | | [Parus major] | |
| PGIRGPFG (SEQ ID | | 99.0 | Y | XP_006812872.1 | PREDICTED: collagen alpha-1(XI) | [1133-1141] |
| 222) | | | | | chain-like [Saccoglossus | |
| | | | | | kowalevskii] | |
| PLGPEGGPPGAPGPEGL | | 99.0 | Y | XP_008049337.1 | PREDICTED: collagen alpha-3(IX) | [85-102] |
| P (SEQ ID 223) | | | | | chain-like [Carlito syrichta] | |
| AHADVVQVQANSLMR | Deamidated | 99.0 | Y | KMM81579.1 | collagen pro alpha-chain | [16-30] |
| (SEQ ID 224) | (N)@11 | | | | precursor [Pseudomonas | |
| | | | | | deceptionensis] | |
| AEEAAAAGALLAARAG | | 99.0 | Y | XP_013966007.1 | PREDICTED: collagen alpha-1(I) | [20-37] |
| AM (SEQ ID 225) | | | | | chain-like [Canis lupus | |
| | | | | | familiaris] | |
| TGPLGPTGLPG (SEQ ID | | 98.9 | Y | XP_008318171.1 | | |
| 226) | | | | | | |
| PPTLVLLTLLVAAVLRC | | 98.7 | Y | XP_008001218.1 | | |
| (SEQ ID 227) | | | | | | |
| GPLGPIGPLG (SEQ ID | | 98.7 | Y | XP_014216795.1 | | |
| 228) | | | | | | |
| FPEDPFR (SEQ ID 229) | | 98.6 | Y | BAL86751.1 | | |
| LIGGLGLI (SEQ ID 230) | | 98.6 | Y | EEE58555.1 | | |
| SGPPGKPGNVGLPGPS | | 98.6 | Y | BAE35005.1 | | |
| GP (SEQ ID 231) | | | | | | |

| | | | | |
|---|---|---|---|---|
| GPKGNDGNPGQP | | 98.4 | Y | XP_018085327.1 |
| (SEQ ID 232) | | | | |
| PGPQGPPGYG (SEQ ID | | 98.2 | Y | XP_006158187.1 |
| 233) | | | | |
| QSQSKGPDPSS (SEQ ID | | 98.1 | Y | XP_014228394.1 |
| 234) | | | | |
| PDGNPGEEGQ (SEQ ID | | 98.1 | Y | EYB81789.1 |
| 235) | | | | |
| GPLGPSGLMGPPGPPG | | 98.1 | Y | XP_015798391.1 |
| P (SEQ ID 236) | | | | |
| MHYVLWCLS (SEQ ID | | 98.0 | Y | XP_012695931.1 |
| 237) | | | | |
| GLPGAPGLPGPPA (SEQ | | 97.9 | Y | XP_019617011.1 |
| ID 238) | | | | |
| QGRPGLPGPAGPKGSK | Gln→pyro- | 97.9 | Y | |
| (SEQ ID 239) | Glu@N- | | | EUB56651.1 |
| | term | | | |
| GGYGPGGGGGGFGP | | 97.7 | Y | EYB96599.1 |
| (SEQ ID 240) | | | | |
| NAGPTGPAGPTVG | | 97.4 | Y | XP_008545826.1 |
| (SEQ ID 241) | | | | |
| YLLIQ (SEQ ID 242) | | 97.1 | Y | XP_012721740.1 |
| GPPGEPGLPGPPGFI | | 97.0 | Y | XP_007950625.1 |
| (SEQ ID 243) | | | | |
| LPGVAGPLGLEG (SEQ | | 96.9 | Y | XP_011472463.1 |
| ID 244) | | | | |
| GLPGLPGPPGPPGPPGI | | 96.9 | Y | XP_008323313.1 |
| PG (SEQ ID 245) | | | | |
| GDPGGPGLPGPLG | | 96.9 | Y | JAB71561.1 |
| (SEQ ID 246) | | | | |
| PAQLPAGASAGDT | | 96.7 | Y | SFI05300.1 |
| (SEQ ID 247) | | | | |
| PGPDGKPGVNGMDGL | | 96.7 | Y | ERG85815.1 |
| (SEQ ID 248) | | | | |
| ERGLPGNPGAPGLPGN | | 96.4 | Y | XP_016082678.1 |
| (SEQ ID 249) | | | | |
| PGMPGLQGMPGNN | Deamidated | 96.3 | Y | XP_006158187.1 |
| (SEQ ID 250) | (Q)@7 | | | |
| LNEMEYPYD (SEQ ID | | 96.3 | Y | XP_007902176.1 |
| 251) | | | | |
| PGERGDPGDTGVPG | | 96.2 | Y | XP_004577673.1 |
| (SEQ ID 252) | | | | |
| PGPEGPPGLPGPPGPC | | 96.2 | Y | CBY23990.1 |
| GP (SEQ ID 253) | | | | |
| TPGTPPIPPQPGYPGSP | | 96.1 | Y | KOX71924.1 |
| G (SEQ ID 254) | | | | |
| TSCLATSQH (SEQ ID | | 96.1 | Y | XP_005010991.1 |
| 255) | | | | |
| GLRGLPGEPGPPGPSGP | | 96.0 | Y | XP_007435381.1 |
| (SEQ ID 256) | | | | |
| FAPRVGFAYDL (SEQ ID | | 96.0 | Y | AEU38398.1 |
| 257) | | | | |
| GDIGPPGPPGLPGESGP | | 95.7 | Y | XP_020373673.1 |
| P (SEQ ID 258) | | | | |
| GHRGYT (SEQ ID 259) | | 95.6 | Y | XP_018522139.1 |
| VNYGGDAGRPFG (SEQ | | 95.3 | Y | EIJ32791.1 |
| ID 260) | | | | |
| GLPGPPGADGLPGPPG | | 95.3 | Y | XP_007888412.1 |
| PA (SEQ ID 261) | | | | |
| GPAGPSGPR (SEQ ID | | 95.3 | Y | XP_008001218.1 |
| 262) | | | | |
| GLPGFTGFPGAPGDPG | | 95.1 | Y | XP_006890385.1 |
| HK (SEQ ID 263) | | | | |
| APDPFR (SEQ ID 264) | | 95.1 | Y | JAR61427.1 |
| IGLPGPQGPEGNKGP | | 95.0 | Y | XP_014612610.1 |
| (SEQ ID 265) | | | | |
| QGASGSPGIPGSPGVQ | | 95.0 | Y | XP_002934910.1 |
| (SEQ ID 266) | | | | |
| RDGLPGIPGLEGE (SEQ | | 94.9 | Y | EYB94204.1 |
| ID 267) | | | | |
| ASGPAGPSGPR (SEQ | | 94.7 | Y | XP_008001218.1 |
| ID 268) | | | | |
| SGPSSGLP (SEQ ID | | 94.6 | Y | NP_776406.1 |
| 269) | | | | |
| QTIEPPRMF (SEQ ID | Gln→pyro- | 94.3 | Y | XP_014216795.1 |
| 270) | Glu@N- | | | |
| | term | | | |
| VDGFRGKPGAP (SEQ | | 94.1 | Y | XP_016364966.1 |
| ID 271) | | | | |

SOP2 papain hydrolysate

| | | | | | | |
|---|---|---|---|---|---|---|
| GLPGLPGPPGPPG(SEQ | | 99.0 | Y | XP_008258403.1 | PREDICTED: collagen alpha-1(IV) | [411-423] |
| ID 272) | | | | | chain [Oryctolagus cuniculus] | |
| GPPGPPGPPGPPG | | 99.0 | Y | XP_008258403.1 | PREDICTED: collagen alpha-1(IV) | [203-215] |
| (SEQ ID 273) | | | | | chain [Oryctolagus cuniculus] | |
| GPPGPPGIPGCN (SEQ | Dioxidation | 99.0 | Y | XP_008258403.1 | PREDICTED: collagen alpha-1(IV) | [115-126] |
| ID 274) | (C)@11 | | | | chain [Oryctolagus cuniculus] | |
| GPAGPSGPRGPA (SEQ | | 99.0 | Y | JAR61427.1 | Collagen alpha-2(V) chain | [1115-1126] |
| ID 275) | | | | | [Fundulus heteroclitus] | |
| GPLGPPGLPGPPG (SEQ | | 99.0 | Y | XP_020469236.1 | | |
| ID 276) | | | | | | |
| GPPGPIGPLGPPG (SEQ | | 99.0 | Y | XP_020469236.1 | collagen alpha-1(XI) chain-like | [1673-1685] |
| ID 277) | | | | | [Monopterus albus] | |
| GPIGPLGPPGPPG (SEQ | | 99.0 | Y | XP_012865680.1 | PREDICTED: collagen alpha-4(IV) | [66-78]; |
| ID 278) | | | | | chain [Dipodomys ordii] | |
| PQGPIGPLGPPGPPG | | 99.0 | Y | XP_012865680.1 | PREDICTED: collagen alpha-4(IV) | [64-78] |
| (SEQ ID 279) | | | | | chain [Dipodomys ordii] | |
| PAVPGPMGPM (SEQ | Oxidation | 99.0 | Y | XP_015818276.1 | PREDICTED: collagen alpha-1(I) | [166-175] |
| ID 280) | (M)@10 | | | | chain-like [Nothobranchius | |
| | | | | | furzeri] | |
| GAPGPIGPPGPPGPPG | | 99.0 | Y | XP_009984740.1 | PREDICTED: collagen alpha- | [190-205] |
| (SEQ ID 281) | | | | | 1(XXVI) chain-like [Tauraco | |
| | | | | | erythrolophus] | |
| GPIGPPGPPGPPGA | | 99.0 | Y | XP_009984740.1 | PREDICTED: collagen alpha- | [193-206] |
| (SEQ ID 282) | | | | | 1(XXVI) chain-like [Tauraco | |
| | | | | | erythrolophus] | |
| GPPGPPGIPGGPGP | | 99.0 | Y | XP_019617011.1 | PREDICTED: collagen alpha-2(IV) | [299-312] |
| (SEQ ID 283) | | | | | chain-like [Branchiostoma | |
| | | | | | belcheri] | |
| LPGAPGLPGPPA (SEQ | | 99.0 | Y | XP_019617011.1 | PREDICTED: collagen alpha-2(IV) | [679-690] |
| ID 284) | | | | | chain-like [Branchiostoma | |
| | | | | | belcheri] | |
| GPPGPIGPPGPPG (SEQ | | 99.0 | Y | CDS37947.1 | collagen type XI alpha 2 | [996-1008] |
| ID 285) | | | | | [Echinococcus multilocularis] | |
| GIPGPIGPPGPPGL | | 99.0 | Y | XP_014441180.1 | collagen alpha-5(IV) chain | [269-282] |
| (SEQ ID 286) | | | | | [Tupaia chinensis] | |
| GIPGPIGPPGPPG (SEQ | | 99.0 | Y | XP_014441180.1 | collagen alpha-5(IV) chain | [269-281] |
| ID 287) | | | | | [Tupaia chinensis] | |
| GPPGPPGLPGPPGA | | 99.0 | Y | XP_018522139.1 | PREDICTED: collagen alpha-2(I) | [1080-1093] |
| (SEQ ID 288) | | | | | chain isoform X2 [Lates | |
| | | | | | calcarifer] | |
| GPLGYPGR (SEQ ID | | 99.0 | Y | XP_018423170.1 | PREDICTED: collagen alpha-1(I) | [799-806] |
| 289) | | | | | chain-like [Nanorana parkeri] | |
| GPIGPLGPPGPPGE | | 99.0 | Y | XP_003439010.1 | collagen alpha-1(II) chain | [474-487] |
| (SEQ ID 290) | | | | | isoform | |
| | | | | | X1 [Oreochromis niloticus] | |
| RAFSLNGNCSYLLLQ | | 99.0 | Y | XP_007902349.1 | PREDICTED: von Willebrand | [1954-1968] |
| (SEQ ID 291) | | | | | factor [Callorhinchus milii] | |
| GPPGMMGPPGME | Oxidation | 99.0 | Y | XP_012696930.1 | PREDICTED: collagen alpha-1(XVI) | [380-391] |
| (SEQ ID 292) | (M)@5 | | | | chain-like [Clupea harengus] | |
| GPAGPPGEPGPIGPPG | | 99.0 | Y | XP_018793992.1 | PREDICTED: collagen alpha chain | [198-214] |
| A (SEQ ID 293) | | | | | CG42342 [Bactrocera latifrons] | |
| PPGERGLPGLPGSPGP | | 99.0 | Y | XP_018049629.1 | PREDICTED: collagen alpha-1(II) | [2085-2100] |
| (SEQ ID 294) | | | | | chain-like [Atta colombica] | |
| AGFPGSPGY (SEQ ID | | 99.0 | Y | ERE91022.1 | collagen alpha-2(IV) chain | [631-639] |
| 295) | | | | | [Cricetulus griseus] | |
| PGMAGPLGPPGPPGPP | | 99.0 | Y | XP_019412231.1 | PREDICTED: collagen alpha-3(V) | [1341-1357] |
| G (SEQ ID 296) | | | | | chain, partial [Crocodylus | |
| | | | | | porosus] | |
| GPIGPPGIPGPGGP | | 99.0 | Y | XP_016150737.1 | PREDICTED: collagen alpha-1(VIII) | [535-548] |
| (SEQ ID 297) | | | | | chain-like [Sinocyclocheilus | |
| | | | | | grahami] | |
| LGTLGMLGIEGIQGILGT | Deamidated | 99.0 | Y | XP_014166856.1 | PREDICTED: collagen alpha-1(VIII) | [28-45] |
| (SEQ ID 298) | (Q)@13 | | | | chain-like, partial [Geospiza | |
| | | | | | fortis] | |
| GLPGPMGPPGLPGSPG | | 99.0 | Y | XP_016773969.1 | collagen alpha-1(XIII) chain | [256-272] |
| P (SEQ ID 299) | | | | | isoform X11 [Pan troglodytes] | |
| GAKGEPAIGYDGA | | 99.0 | Y | CEF70044.1 | Collagen alpha-1(IV) chain | [156-167] |
| (SEQ ID 300) | | | | | [Strongyloides ratti] | |
| GLPGPPGPIGPPGE | | 99.0 | Y | XP_002429374.1 | collagen alpha-1 precursor, | [997-1010] |
| (SEQ ID 301) | | | | | putative [Pediculus humanus | |
| | | | | | corporis] | |
| RDGLPGLRGDQGPLG | Deamidated | 99.0 | Y | XP_018114574.1 | PREDICTED: collagen alpha-1(VII) | [1929-1943] |
| (SEQ ID 302) | (Q)@11 | | | | chain-like isoform X2 [Xenopus | |
| | | | | | laevis] | |
| IGAAGYPGMPGRLGAR | Oxidation | 99.0 | Y | XP_011554965.1 | PREDICTED: collagen alpha-1(IV) | [968-992] |
| GPFGLKGER (SEQ ID | (M)@9 | | | | chain [Plutella xylostella] | |
| 303) | | | | | | |
| GIPGLPGPPGPPG (SEQ | | 99.0 | Y | XP_007889000.1 | PREDICTED: collagen alpha- | [1003-1015] |
| ID 304) | | | | | 1(XVIII) chain [Callorhinchus | |
| | | | | | milii] | |
| GLPGPIGPPGPPG (SEQ | | 99.0 | Y | XP_013049925.1 | PREDICTED: collagen alpha-1(IV) | [1247-1259] |
| ID 305) | | | | | chain [Anser cygnoides | |
| | | | | | domesticus] | |
| GLPGPIGPPGPPGL | | 99.0 | Y | XP_013049925.1 | PREDICTED: collagen alpha-1(IV) | [1247-1260] |
| (SEQ ID 306) | | | | | chain [Anser cygnoides | |
| | | | | | domesticus] | |
| PLGRDASDQHIIEVVL | Deamidated | 99.0 | Y | XP_004073919.1 | collagen alpha-2(IX) chain | [515-530] |
| (SEQ ID 307) | (Q)@9 | | | | [Oryzias latipes] | |
| GPIGLPGPPGPPG (SEQ | | 99.0 | Y | XP_004610907.1 | PREDICTED: collagen alpha-3(IV) | [1217-1229] |
| ID 308) | | | | | chain [Sorex araneus] | |
| GPMGPPGPQGPPGQG | Deamidated | 99.0 | Y | XP_008684503.1 | PREDICTED: collagen alpha- | [461-479] |
| SPGP (SEQ ID 309) | (Q)@9; | | | | 1(XXVIII) chain [Ursus maritimus] | |
| | Deamidated | | | | | |
| | (Q)@14 | | | | | |
| GIVMGTSH (SEQ ID | | 99.0 | Y | KYF83355.1 | glycosyl hydrolase [Sorangium | [332-339] |
| 310) | | | | | cellulosum] | |
| LGADDKDAFGFDAGNG | | 99.0 | Y | WP_007392357.1 | Hep/Hag repeat protein, partial | [2703- |
| (SEQ ID 311) | | | | | [Megasphaera sp. UPII 135-E] | 2717]; |
| PGNPGPMGPPGLP | | 98.6 | Y | XP_005306644.1 | | |
| (SEQ ID 312) | | | | | | |
| GRDGEKGYPGLPGEAG | | 98.5 | Y | XP_013307530.1 | | |
| LP (SEQ ID 313) | | | | | | |
| GLPGPPGPPGPMGP | | 98.4 | Y | XP_018606315.1 | | |
| (SEQ ID 314) | | | | | | |
| LPGLQGPPGFLGLPGLD | | 98.4 | Y | XP_015983069.1 | | |
| GNMGL (SEQ ID 315) | | | | | | |
| GPPGPPGPPGPPGE | | 98.3 | Y | XP 008258403.1 | | |
| (SEQ ID 316) | | | | | | |
| ERGPLGPPGLPGFT | Glu→pyro- | 98.2 | Y | XP_008258403.1 | | |
| (SEQ ID 317) | Glu@N- | | | | | |
| | term | | | | | |
| GPQGPAGPSGQPGL | | 98.2 | Y | XP_018093746.1 | | |
| (SEQ ID 318) | | | | | | |
| VAIAVHSQDIT (SEQ ID | | 98.1 | Y | JAQ92290.1 | | |
| 319) | | | | | | |
| PGSPGAAGERGLRGQT | | 98.1 | Y | XP_013073568.1 | | |
| GMPGMP (SEQ ID | | | | | | |
| 320) | | | | | | |
| EPGLMGLPGVAGHVG | | 98.1 | Y | KMQ93415.1 | | |
| (SEQ ID 321) | | | | | | |
| AGPAGIAGPR (SEQ ID | | 98.0 | Y | XP_015818276.1 | | |
| 322) | | | | | | |
| PPGPTGSPGPIGPSGPP | | 97.9 | Y | KTG43214.1 | | |
| G (SEQ ID 323) | | | | | | |
| PIGGPGAPGVPGP | | 97.9 | Y | XP_008430496.1 | | |
| (SEQ ID 324) | | | | | | |
| YPGEAGCPGEAGLPGA | | 97.9 | Y | CAA75877.1 | | |
| KGLT (SEQ ID 325) | | | | | | |

| LGPAGLPGPHQ (SEQ | | 97.8 | Y | XP_014905999.1 |
|---|---|---|---|---|
| ID 326) | | | | |
| GDIGPPGPPGLPGESGP | | 97.8 | Y | XP_020373672.1 |
| P (SEQ ID 327) | | | | |
| LIGPAGPPGPPGEPA | | 97.7 | Y | XP_019764717.1 |
| (SEQ ID 328) | | | | |
| LPGTVGLPGTIGPPGV | | 97.7 | Y | XP_019905544.1 |
| (SEQ ID 329) | | | | |
| PGSPGPVGPPGM (SEQ | | 97.5 | Y | XP_008115785.1 |
| ID 330) | | | | |
| GPIGPLGPPGLPG (SEQ | | 97.5 | Y | XP_020469236.1 |
| ID 331) | | | | |
| GPLGPQGPIGPLGPPG | | 97.5 | Y | XP_012865680.1 |
| (SEQ ID 332) | | | | |
| GAIGERGE (SEQ ID | | 97.0 | Y | XP_017324879.1 |
| 333) | | | | |
| YPGQPGTPGKPGQP | | 96.8 | Y | XP_018897252.1 |
| (SEQ ID 334) | | | | |
| PAGPTGSQGLPGP | | 96.8 | Y | XP_009326101.1 |
| (SEQ ID 335) | | | | |
| PHSASASSVTAS (SEQ | | 96.6 | Y | |
| ID 336) | | | | XP_013959841.1 |
| AGPVGPMGP (SEQ ID | | 96.4 | Y | XP_018606315.1 |
| 337) | | | | |
| RGMPGPPGT (SEQ ID | | 96.1 | Y | XP_013919176.1 |
| 338) | | | | |
| GPAGDPGPQ (SEQ ID | | 96.1 | Y | CDS37947.1 |
| 339) | | | | |
| PGLSGPKGEKGDFGPQ | | 96.1 | Y | XP_018102242.1 |
| GPPGT (SEQ ID 340) | | | | |
| GASGQDGRDGIGPPGP | | 96.1 | Y | JAO33091.1 |
| (SEQ ID 341) | | | | |
| DKGEPGGPGADGAPG | | 96.1 | Y | XP_012583529.1 |
| (SEQ ID 342) | | | | |
| GPIGPLGPPGLMGP | | 96.0 | Y | XP_004281573.1 |
| (SEQ ID 343) | | | | |
| GPSGIPGLPGNPG (SEQ | | 96.0 | Y | NP_493702.1 |
| ID 344) | | | | |
| PGATGSQGIQGPQ | | 96.0 | Y | WP_061673050.1 |
| (SEQ ID 345) | | | | |
| TPGPKGEPGQPGFP | | 95.9 | Y | XP_018559311.1 |
| (SEQ ID 346) | | | | |
| PAGYPGPRGAPGPVGP | | 95.9 | Y | NP_500607.2 |
| PGVA (SEQ ID 347) | | | | |
| PGITGPAGPAGPLG | | 95.9 | Y | SKZ79532.1 |
| (SEQ ID 348) | | | | |
| PVGMEYKE (SEQ ID | | 95.8 | Y | XP_009286777.1 |
| 349) | | | | |
| TGDTGPQGPKGDTGA | | 95.8 | Y | AMO43684.1 |
| QGPQ (SEQ ID 350) | | | | |
| PGIPGAPGHPGA (SEQ | | 95.5 | Y | XP_012134692.1 |
| ID 351) | | | | |
| PAGPTGPPGPIGPSGPP | | 95.5 | Y | XP_005165282.1 |
| G (SEQ ID 352) | | | | |
| DGAESSLAGL (SEQ ID | | 95.2 | Y | KUF16894.1 |
| 353) | | | | |
| GPAGGPGVDGYPGL | | 95.1 | Y | XP_017293604.1 |
| (SEQ ID 354) | | | | |
| GPIGLPGPPGPPGF | | 95.1 | Y | XP_004610907.1 |
| (SEQ ID 355) | | | | |
| ERGLPGFI (SEQ ID 356_ | Glu→pyro- | 95.0 | Y | XP_005801088.1 |
| | Glu@N- | | | |
| | term | | | |
| GPIGPPGPPGPPG (SEQ | | 94.8 | Y | XP_009984740.1 |
| ID 357) | | | | |
| GPLGPPGPPGPPG | | 94.8 | Y | XP_019412231.1 |
| (SEQ ID 358) | | | | |
| GILGTLGMLGIEGIQGIL | | 94.6 | Y | XP_014166856.1 |
| GTL (SEQ ID 359) | | | | |
| SNADQRSIEGT (SEQ ID | | 94.1 | Y | XP_007889000.1 |
| 360) | | | | |

| | | | | |
|---|---|---|---|---|
| PGQPGLPGLPGPPGAP | | 94.0 | Y | XP_007907447.1 |
| (SEQ ID 361) | | | | |
| ERGPLGPPGLPGF (SEQ | Glu→pyro- | 94.0 | Y | XP_008258403.1 |
| ID 362) | Glu@N- | | | |
| | term | | | |

¹Amino acid nomenclature: A, ala, alanine; C, cys, cysteine; D, asp, aspartic acid; E, glu, glutamic acid; F, phe, phenylalanine; G, gly, glycine; H, his, histidine; I, Ile, isoleucine; K, lys, lysine; L, leu, leucine; M, met, methionine; N, asn, asparagine; P, pro, proline; Q, gln, glutamine; R, arg, arginine; S, ser, serine; T, thr, threonine; V, val, valine; W, trp, tryptophan; Y, tyr; tyrosine; X, undetermined amino acid.
²Accession numbers of protein of origin according to NCBI non-redundant database
³Percentage of confidence of the assigned sequence using MASCOT search engine

TABLE 12
amino acids identified in the retentate and 10-kDa fractions
recovered using MWCO of gelatine hydrolysates generated
using papain and alcalase. Gelatine is a denatured protein
derived by partial hydrolysis of collagen.

| | | Gelatine | Gelatine |
|---|---|---|---|
| Amino acid | Gelatine | hydrolysate | hydrolysate |
| (g/Kg): | extract | papain | alcalase |
| Cysteic acid | 0.247 | 3.40 | 3.66 |
| Taurine | 5.05 | 3.04 | 2.11 |
| Methionine | 24.9 | 23.1 | 20.8 |
| sulfone | | | |
| Asp | 49.5 | 51.3 | 47.9 |
| Thr | 22.1 | 23.8 | 21.7 |
| Ser | 42.4 | 47.9 | 41.7 |
| Glu | 80.4 | 84.6 | 78.7 |
| Gly | 202.2 | 205.6 | 190.4 |
| Ala | 77.8 | 80.4 | 72.7 |
| Cysteine | 4.28 | 3.72 | 2.77 |
| Val | 16.4 | 18.3 | 19.8 |
| Ile | 9.99 | 8.83 | 11.2 |
| Leu | 18.8 | 19.7 | 20.7 |
| Tyr | 2.86 | 5.87 | 5.69 |
| Phe | 18.5 | 20.1 | 18.7 |
| His | 11.9 | 11.8 | 11.1 |
| Lys | 28.0 | 32.3 | 30.5 |
| NH3 | 6.99 | 5.87 | 6.52 |
| Arg | 68.9 | 69.7 | 66.7 |
| Pro | 81.8 | 81.6 | 76.6 |
| SUM (g/Kg) | 773.0 | 800.9 | 749.9 |

### Discussion

Yields and Proximate Composition of Gelatines

It is known that numerous factors, including fish species, quality of by-products, the pre-treatment steps and the extraction time and temperature can influence the yield of gelatine produced. In this study, the by-product consisted of a mixture of fish skin, bones and muscle left after industrial processing of blue whiting into surimi products. As can be seen from Table 8, gelatines extracted using SOP1, SOP2 and SOP4 procedures produced similar yields of gelatine (around 5%), while the yield of gelatine obtained using SOP3 was significantly lower. SOP1 and SOP2 gelatine samples also had high protein and low ash contents compared to gelatines generated using SOP3 and SOP4. Notably, the LGC of SOP1, SOP2 and SOP4 gelatines was approximately 2% (w/v), while gelatine generated using SOP3 had gelling properties only at significantly higher (8%, w/v) concentrations. Solutions of SOP1 and SOP2 gelatines had pH values closer to neutral, while gelatines generated using SOP3 and SOP4 had pH values in the range of X-X. The water activities of the samples after freeze-drying were comparable to the a_{w} of bovine and porcine gelatines. This indicates that the gelatines are safe for use. Most pathogenic bacteria require a_{w} value of above 0.91, and a product can be considered microbiologically stable if its Aw is under 0.6 (32). A decrease in L (lightness) parameter indicates a general darker colour, and it can be seen that SOP1, SOP2 and SOP4 samples compare favourably to the control gelatines (Table 9). The notable exception is the enzymatically pre-treated SOP3 gelatine, which shows the lowest L value of all samples, as well as highest value of b (yellowness/blueness) parameter corresponding to its strong yellow coloration. C (Chroma) value is a tool for expressing the colour intensity, and from the calculated C values it can be clearly seen that SOP3 gelatine had the most intense colouring. The SDS-PAGE (FIG. 8) results show that SOP1, SOP2 and SOP4 gelatine samples have similar protein patterns and consist of γ-, β- and α-protein chains, with other minor bands that indicate varying levels of sample hydrolysis. In the SOP3 gelatine sample, however, absence of the heavy γ-chain and strong presence of lower molecular weight bands indicate an increased level of protein hydrolysis.

Previously, Jellouli et al. (33) reported a yield of 5.67% gelatine from fish skins of the grey triggerfish (Balistes capriscus) when pre-treatment with 0.2 mol/L of NaOH and 0.05 mol/L of acetic acid was used, while others including Balti et al. (34), reported a lower gelatine yield (2.21%) when protease enzyme was used as a pre-treatment in the case of cuttlefish (Sepia officinalis) skin gelatine. According to the Gelatine Handbook (35) specifications, edible gelatine should normally contain between 0.3-2% ash, depending on the production process, and ash content of the samples in this study was higher than these limits. It is known that the colour of fish sourced gelatine may vary widely, depending on the fish species, raw material, pre-treatment and extraction process, although it generally does not affect its functional properties. Recently, Kittiphattanabawon et al. (30) have investigated the influence of extraction conditions on the properties of gelatine obtained from Clown featherback (Chitala ornata). Their results showed a decrease in MW bands (γ-, β- and α1-chains) intensity with the increase of extraction time and temperature. In our study, the extraction temperature and time were kept constant in all SOPs, so it can be concluded that pre-treatment conditions also show a pronounced effect on the protein distribution in extracted fish gelatines. Limitations concerning the applications of fish gelatine are mainly due to lower gel strengths and stability and lower gelling and melting temperatures in comparison to mammalian-sourced gelatines (1). These differences are especially prominent in the case of gelatines extracted from cold-water fish species, which are known to contain smaller number of imino acids (proline and hydroxyproline) and a larger quantity of higher non-polar amino acids (36). The properties of gelatines produced during our experiments are in accordance with literature reports comparing cold-water fish gelatines to warm-water species and mammalian sources [(1), (37)]. Therefore, non-modified gelatines obtained from cold-water fish species, such as blue whiting, may not be successfully used in applications where gelling at room temperature is required (e.g. production of jelly desserts).

Techno-Functional Properties of Gelatines

WHC refers to the ability of protein to absorb water and retain it against a gravitational force within the protein matrix (38). The WHC values of the SOP1, SOP2, SOP3 and SOP4 gelatines (FIG. 9) were significantly lower than the controls bovine and porcine-derived gelatines. Ability to retain water is largely dependent on the hydrophilicity of the gelatine, with gelatines of mammalian origin being generally richer in hydrophilic amino acids, mainly proline and hydroxyproline, compared to fish sourced gelatines. Also, the low WHC values observed may be partially explained by high solubility of fish gelatine in water and the inability of the produced samples to form gel at room temperature.

SOP1 gelatine had the highest OHC values for both sunflower and olive oil. SOP2 and SOP4 samples had comparable OHC, while SOP3 sample had the lowest values for both OHC and WHC of all the produced samples. Fat binding capacity depends on the degree of exposure of the hydrophobic residues inside gelatine, and therefore gelatines obtained from cold water fish species, richer in hydrophobic amino acid residues, may show a better OHC and lower WHC compared to mammalian gelatines.

Results in this study show significant differences in WHC and OHC values, implying that the products obtained from blue whiting belong to the cold water fish gelatine type, and could be potentially be utilized as food emulsifiers. The solubility of all gelatine samples (FIG. 10) in water was high for all samples, with SOP1 and SOP2 samples being the most soluble (solubility over 80%) at all tested pH levels. SOP3 and SOP4 samples also had high solubility, which was comparable to bovine and porcine gelatines.

The FC (FIG. 10) of all investigated gelatine solutions was highest at pH=2 and was comparable to control gelatine values. At all other tested pH values, the FC of fish gelatine samples (SOP1, SOP2, SOP3 and SOP4) and control gelatines were comparable and were less than 50%. SOP3 derived gelatine had a significantly lower FC than the other samples. This is in accordance with literature findings which indicate that foam stability of protein solutions is usually positively correlated with the molecular weight of peptides. FS values after 60 min in FIG. 10 show that the foam of fish gelatine samples had lower stability in comparison to control samples. The hydrophobic areas in the gelatine peptide chain influence its amphoteric characteristics, enabling it to be used as an emulsifying agent in various applications (cooking, manufacture of desserts, oil-in-water emulsions such as low-fat margarine, salad dressings, milk cream and others). The 1% (w/v) fish gelatine solutions in our experiment have produced significant emulsifying activity (EA) and emulsifying stability (ES) with sunflower oil (FIG. 11) over the tested ranges of pH values (2-10) compared to the controls. EA of all gelatine samples was highest at pH=2. Bovine and porcine gelatines, had significantly lower EA and ES at pH=10 and pH=4, respectively.

On the basis of the results obtained in this study, it can be concluded that pre-treatment has a considerable influence on the properties of gelatine extracted from Blue whiting by-product consisting of skin, bones and meat. The combination of alkaline, diluted mineral acid and diluted acetic acid pre-treatment in the case of SOP1 and SOP2 samples has proven to yield gelatine with best physical and chemical properties and most optimal techno-functional characteristics. Using mild organic acid (citric acid) for the tested period of time has produced a highest yield of gelatine with best colour characteristics, albeit with high mineral content and less optimal techno-functional characteristics. In the case of SOP3 pre-treatment, which consisted of enzymatic hydrolysis (using alcalase) and subsequent treatment with mineral acid, the gelatine produced had notably inferior physical and chemical characteristics, lower yield and significantly impaired techno-functional properties. This indicates an excessive level of collagen hydrolysis if alcalase is used, which was further substantiated by the results of SDS-PAGE protein analysis.

Enzyme Hydrolysis

Enzymatic hydrolysis of food by-products is an interesting strategy for by-product utilisation. The interest of numerous researchers is particularly focused on hydrolysis of marine processing by-products, since it has been shown that many of the hydrolysis products possess various biological activities, such as antioxidant, antihypertensive, antibacterial and anti-obesity effects. Based on the overall properties of the gelatines developed in this study, gelatines generated using SOP1 and SOP2 were chosen for further hydrolysis using alcalase, and papain enzymes. Determination of the degree of hydrolysis (DH) is an important step in characterization of protein hydrolysates for use in functional food products. Also, it is known that DH has an important role in ACE-I inhibitory activity, with higher DH values principally indicating better potential bioactivity.

Although optimal conditions for the enzymatic hydrolysis were applied, the attained degree of hydrolysis differed significantly between the samples. Hydrolysis of gelatine using resulted in a DH values of 20 The DH of gelatine using alcalase was 2.54±0.12% for SOP2 derived gelatine.

Bioactivity of Papain Hydrolysates

The in vitro ACE-I inhibitory activities of the alcalase and papain hydrolysates were calculated at a concentration of 1 mg/ml and Captopril® (0.5 mg/ml) was used as positive control. The increase in bioactivity of the low molecular weight fraction is in agreement with previously reported findings implying that lower molecular weight peptides are principally responsible for ACE-I inhibition in protein hydrolysates. When alcalase was used for hydrolysis of gelatines, the differences between the ACE-I inhibitory activity of whole hydrolysates and ≤3 KDa peptide fractions were significant. This was however not the case when papain was used for hydrolysis, where whole hydrolysates had activity comparable to the purified ≤3 KDa peptide fractions. This can be explained by more complete protein hydrolysis when papain was used, which is shown by higher calculated DH values.

MS Analysis of Peptides

The ≤3 KDa peptide fractions of the papain hydrolysates were determined to have the highest ACE-I inhibitory activity and were therefore selected for MS analysis in order to determine their peptide composition. All of the determined peptides (81 in SOP1 sample and 90 in SOP2 sample) were found to be unique when compared to peptides in the BIOPEP peptide database. The amino acid composition of the hydrolysates was similar to the intact gelatine samples, and the results show that most abundant amino acids within peptide sequences were glycine (190.4 to 210.6 g/Kg), glutamic acid (78.7 to 84.6 g/Kg), alanine (72.7 to 80.5 g/Kg) and proline (76.6 to 86.7 g/Kg). It has been suggested that residues with bulky hydrophobic side-chains (proline, tryptophan, tyrosine and phenylalanine) are the most effective amino acids for inhibition of ACE-I in dipeptides, but also the N-terminal side of peptide inhibitors may benefit from small, as well as hydrophobic side chains such as leucine, valine and isoleucine (45). The peptides that were identified with ≥99.0% analytical confidence (37 in SOP1 and 39 in SOP2 sample) were checked against FASTA sequences of the corresponding proteins used for confirmation in the NCBI protein database. The structure of most identified peptides (36 in SOP1 sample and 37 in SOP2 sample) was confirmed in this manner, and the corresponding proteins show a strong correlation between amino acid sequence of identified peptides and alpha-chain collagen sequences.

The identified peptides were then searched for the potential bioactive fragments using the BIOPEP (46) analysis tools. The search resulted in numerous identified fragments with ACE-I inhibitory activity, such tripeptides GPL, PGL, GPM, DGL and dipeptides PP, PG, GG, AG, and IG in the tested peptide sequences.

### Example 3: Control Recipe for Dog Biscuit

49.5 g wheat flour
1.0 g sodium chloride
7.4 g sucrose
0.8 g sodium hydrogen carbonate
1.8 g sodium phosphate (dibasic)
9.9 g sunflower oil
29.6 g water

Test Sample 1

Substitute 10 g of flour with 10 g 3 kDa fraction

Test Sample 2

Substitute 10 g of flour with 10 g 10 kDa fraction

Test Sample 3

Substitute 10 g of flour with 10 g retentate fraction

Methodology

Mix and homogenize ingredients, add water and oil and knead to obtain soft dough. The dough was rested for 15 to 20 minutes, rolled on a plate to approximately 0.5 cm thickness and cut. The biscuits were baked at 140° C. for 45 minutes in a convection oven.

Results

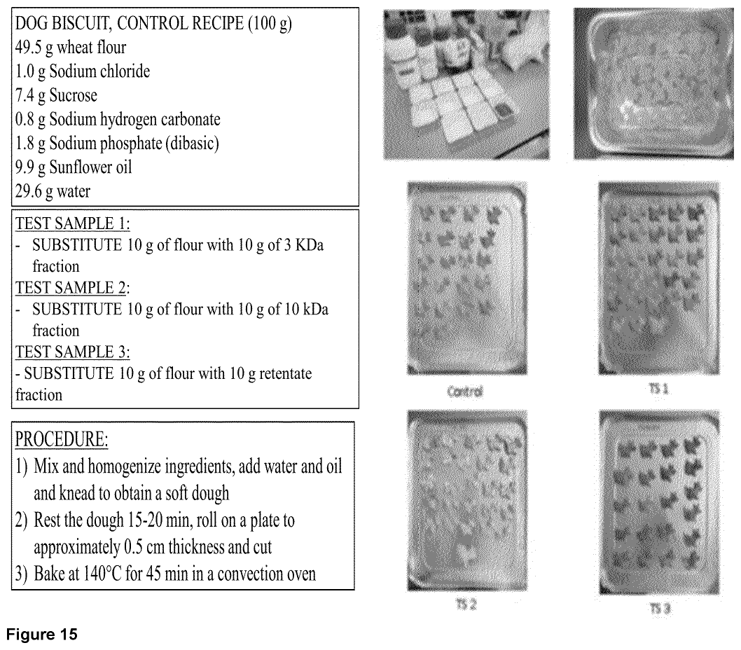

FIG. 15 illustrates a method for making a biscuit of the invention. A rich brown colour was achieved during baking with bloodwaters but not with blue-whiting derived proteins from skin and bones. The mixture necessitates the use of lower than normal batch water temperatures during baking to achieve a “spring” or rise in the biscuit.

The colour of the biscuit was tested:

For the gelatine hydrolysate—

It can be seen that values of L (lightness) parameter ranged from 82.7 (±3.14) for SOP3 sample to 95.3 (±0.73) in the case of SOP1 sample. The b parameter (yellowness/blueness) showed a wide range of determined values, between 2.58 (±0.85) and 29.5 (±5.69) (SOP4 and SOP3 samples, respectively); similarly to calculated C value (chroma) of the samples, which was in the range between 2.62 (SOP4) and 29.62 (SOP3). A parameter (greenness/redness) of all measured samples had negative value, indicating a slight colour shift toward greenness.

### Example 4

Anti-Hypertensive Effect in Dogs In Vivo.

The gelatine samples were generated from the raw material using SOP2. 250 g (×3) of whole, blue-whiting H&G material was minced in a food blender for 30 seconds and subsequently added to 250 ml of distilled, deionised water. The slurry obtained was heated for 10 min at 80° C. to inactivate endogeneous enzymes. Hydrolysis was carried out at 140 rpm, 55° C., pH 8 using the enzyme Alcalase® which was added to the H&G material at the ratio of 1:100 (w:v). Hydrolysis was carried out for 4 hours. The volume of 0.1 M NaOH used to adjust the pH of the hydrolysate to 6.5 was noted and hydrolysis was terminated by heating at 95° C. for 10 min to inactivate the added enzyme. The hydrolysate was cooled to room temperature, the hydrolysate slurry was poured into trays, frozen and then freeze-dried using a Labconco freeze drier (Labconco corporation, USA) and a set programme for 48 h. The freeze-dried hydrolysates were weighed to calculate yield by-product H&G per batch and hydrolysates were subsequently analysed for protein, lipid and other components.

Formulation and baking conditions for PAW dog biscuits containing H& G whole alcalase hydrolysate or H&G gelatine papain hydrolyate or mussel whole papain hydrolyate at a concentration of 10%.

Control Recipe (for 600 g of Dough)
Flour: 268 g
Sunflower oil: 50 g
Sugar: 37 g
Water: 145 g
Additional flour (for rolling and kneading): 100 g

Experiment Recipe (for 600 g of Dough, Around 10% Added Protein):
Flour: 243 g
X blood-water: 25 g
Sunflower oil: 50 g
Sugar: 37 g
Water: 145 g
Additional flour (for rolling and kneading): 100 g

Baking Procedure:

Mix ingredients; knead for 3-5 minutes. Roll the dough using automatic sheeter (to approx 3.5 mm thickness) and cut out biscuits using cutter. Put on non-stick baking tray and bake in pre-heated oven at 150° C. for 40 min. Water loss factor: approximately =0.76

Cookies without test ingredient marked as “Control” and cookies with test ingredients named M Cookies (Mussel papain hydrolysate cookies) and H&G cookies (Blue whiting H&G hydrolysate cookies) were used in in vivo dog studies.

The study was performed at an approved pet kennel in the Netherland (Kennel De Morgenstond). All tests carried out were non-invasive and did not harm the animals or the wellbeing of the animal. The study was executed with ten senior dogs (8-13 years old) over three weeks (21 days). A “matched pair design” format was used in this study. Experimental protocols with the animals is attached in Appendix A. 1 (2+5, where 2 days adjustment on new feed/snack, 5 days recording period). For the first week, dogs are fed with the control cookies. After the first 7 days, dogs were then on a break for 2 days and were then in the new test for 5 days. During the course of the whole study, dogs were given 500 g of Pedigree kibbles (The proximate analysis of the kibble feed is shown in table 4.1). During the control period, in addition to food, each dog was offered 2 control biscuits per day. The same procedure was repeated for the test diets.

Results

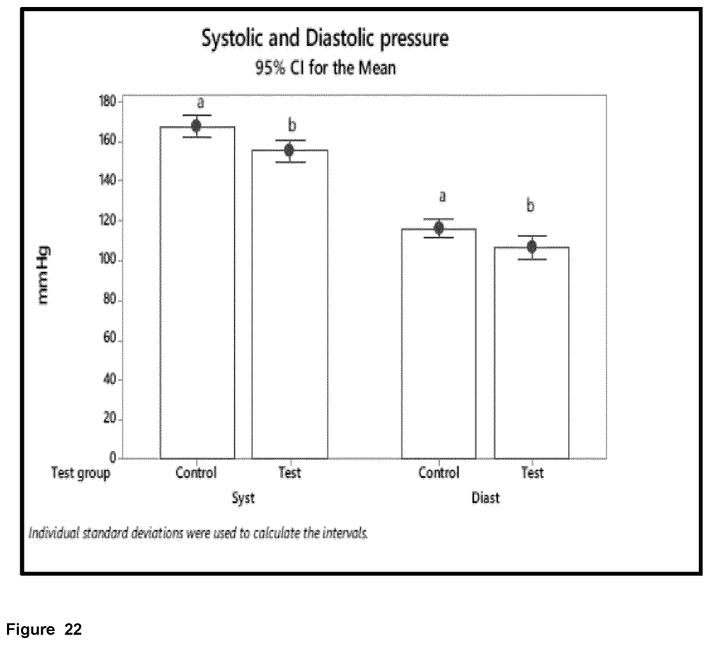

Measurement of blood pressure of the dogs showed a significant decrease in BP when the H&G (headed and gutted) gelatine hydrolysate cookie was given to dogs compared to the control (FIG. 22). Alcalase® was used as the enzyme to obtain the hydrolysate. The dogs were tested and fed the cookie over 5 days.

### REFERENCES

1. Sinthusamran, S.; Benjakul, S.; Kishimura, H., Characteristics and gel properties of gelatin from skin of seabass (Lates calcarifer) as influenced by extraction conditions. Food Chemistry 2014, 152, 276-284.
2. Haddar, A.; Bougatef, A.; Balti, R.; Souissi, N.; Koched, W.; Nasri, M., Physicochemical and functional properties of gelatin from tuna (Thunnus thynnus) head bones. Journal of Food & Nutrition Research 2011, 50.
3. Lassoued, I.; Jridi, M.; Nasri, R.; Dammak, A.; Hajji, M.; Nasri, M.; Barkia, A., Characteristics and functional properties of gelatin from thornback ray skin obtained by pepsin-aided process in comparison with commercial halal bovine gelatin. Food Hydrocolloids 2014, 41, 309-318.
4. Uhlmann, S. S., The European Landing Obligation: Reducing Discards in Complex, Multi-Species and Multi-Jurisdictional Fisheries. Springer: 2019.
5. Halim, N. R. A.; Yusof, H. M.; Sarbon, N. M., Functional and bioactive properties of fish protein hydolysates and peptides: a comprehensive review. Trends in Food Science & Technology 2016, 51, 24-33.
6. Hayes, M.; Mora, L.; Hussey, K.; Aluko, R. E., Boarfish protein recovery using the pH-shift process and generation of protein hydrolysates with ACE-I and antihypertensive bioactivities in spontaneously hypertensive rats. Innovative Food Science & Emerging Technologies 2016, 37, 253-260.
7. Garcia-Moreno, P. J.; Espejo-Carpio, F. J.; Guadix, A.; Guadix, E. M., Production and identification of angiotensin I-converting enzyme (ACE) inhibitory peptides from Mediterranean fish discards. Journal of Functional Foods 2015, 18, 95-105.
8. Elavarasan, K.; Shamasundar, B. A.; Badii, F.; Howell, N., Angiotensin I-converting enzyme (ACE) inhibitory activity and structural properties of oven-and freeze-dried protein hydrolysate from fresh water fish (Cirrhinus mrigala). Food chemistry 2016, 206, 210-216.
9. López-Barrios, L.; Gutiérrez-Uribe, J. A.; Serna-Saldivar, S. O., Bioactive peptides and hydrolysates from pulses and their potential use as functional ingredients. Journal of food science 2014, 79, R273-R283.
10. Koli, J. M.; Basu, S.; Nayak, B. B.; Patange, S. B.; Pagarkar, A. U.; Gudipati, V., Functional characteristics of gelatin extracted from skin and bone of Tiger-toothed croaker (Otolithes ruber) and Pink perch (Nemipterus japonicus). Food and bioproducts processing 2012, 90, 555-562.
11. Tabarestani, H. S.; Maghsoudlou, Y.; Motamedzadegan, A.; Mahoonak, A. R. S., Optimization of physico-chemical properties of gelatin extracted from fish skin of rainbow trout (Onchorhynchusmykiss). Bioresource Technology 2010, 101, 6207-6214.
12. Khiari, Z.; Rico, D.; Martin-Diana, A. B.; Barry-Ryan, C., Comparison between gelatines extracted from mackerel and blue whiting bones after different pre-treatments. Food chemistry 2013, 139, 347-354.
13. Niu, L.; Zhou, X.; Yuan, C.; Bai, Y.; Lai, K.; Yang, F.; Huang, Y., Characterization of tilapia (Oreochromis niloticus) skin gelatin extracted with alkaline and different acid pretreatments. Food Hydrocolloids 2013, 33, 336-341.
14. Horwitz, W.; Chichilo, P.; Reynolds, H., Official methods of analysis of the Association of Official Analytical Chemists. Official methods of analysis of the Association of Official Analytical Chemists. 1970.
15. Kolar, K., Gravimetric determination of moisture and ash in meat and meat products: NMKL interlaboratory study. Journal of AOAC International (USA) 1992.
16. Barbosa-Cánovas, G. V.; Fontana Jr, A. J.; Schmidt, S. J.; Labuza, T. P., Water activity infoods: fundamentals and applications. John Wiley & Sons: 2008; Vol. 13.
17. Safety. https://www.gelatine.org/gelatine/safety.html files/153/safety.html
18. Ogunwolu, S. O.; Henshaw, F. O.; Mock, H.-P.; Santros, A.; Awonorin, S. O., Functional properties of protein concentrates and isolates produced from cashew (Anacardium occidentale L.) nut. Food chemistry 2009, 115, 852-858.
19. Musso, Y. S.; Salgado, P. R.; Mauri, A. N., Smart edible films based on gelatin and curcumin. Food hydrocolloids 2017, 66, 8-15.
20. Garcia-Vaquero, M.; Lopez-Alonso, M.; Hayes, M., Assessment of the functional properties of protein extracted from the brown seaweed Himanthalia elongata (Linnaeus) SF Gray. Food Research International 2017, 99, 971-978.
21. Neto, V. Q.; Narain, N.; Silva, J. B.; Bora, P. S., Functional properties of raw and heat processed cashew nut (Anacardium occidentale, L.) kernel protein isolates. Food/Nahrung 2001, 45, 258-262.
22. Bencini, M. C., Functional properties of drum-dried chickpea (Cicer arietinum L.) flours. Journal of Food Science 1986, 51, 1518-1521.
23. Lafarga, T.; Hayes, M., Effect of pre-treatment on the generation of dipeptidyl peptidase-IV-and prolyl endopeptidase-inhibitory hydrolysates from bovine lung. Irish journal of agricultural and food research 2017, 56, 12-24.
24. Adler-Nissen, J., Enzymatic Hydrolysis of Food Protein. Elsevier Applied Science Publishers LTD, London, UK: 1986; p 451.
25. Hill, R. L., Hydrolysis of proteins. In Advances in protein chemistry, Elsevier: 1965; Vol. 20, pp 37-107.
26. Shilov, I. V.; Seymour, S. L.; Patel, A. A.; Loboda, A.; Tang, W. H.; Keating, S. P.; Hunter, C. L.; Nuwaysir, L. M.; Schaeffer, D. A., The Paragon Algorithm, a next generation search engine that uses sequence temperature values and feature probabilities to identify peptides from tandem mass spectra. Molecular & cellular proteomics: MCP 2007, 6, 1638-55.
27. Kaewruang, P.; Benjakul, S.; Prodpran, T.; Nalinanon, S., Physicochemical and functional properties of gelatin from the skin of unicorn leatherjacket (Aluterus monoceros) as affected by extraction conditions. Food Bioscience 2013, 2, 1-9.
28. da Trindade Alfaro, A.; Balbinot, E.; Weber, C. I.; Tonial, I. B.; Machado-Lunkes, A., Fish gelatin: Characteristics, functional properties, applications and future potentials. Food Engineering Reviews 2015, 7, 33-44.
29. Jakhar, J. K.; Reddy, A. D.; Maharia, S.; Devi, H. M.; Reddy, G. V. S.; Venkateshwarlu, G., Characterization of fish gelatin from Blackspotted Croaker (Protonibea diacanthus). Archives of Applied Science Research 2012, 4, 1353-1358.
30. Kittiphattanabawon, P.; Benjakul, S.; Sinthusamran, S.; Kishimura, H., Gelatin from clown featherback skin: Extraction conditions. LWT-Food Science and Technology 2016, 66, 186-192.
31. Hema, G. S.; Joshy, C. G.; Shyni, K.; Chatterjee, N. S.; Ninan, G.; Mathew, S., Optimization of process parameters for the production of collagen peptides from fish skin (Epinephelus malabaricus) using response surface methodology and its characterization. J Food Sci Technol 2017, 54, 488-496.
32. Abbas, K. A.; Saleh, A. M.; Mohamed, A.; Lasekan, O., The relationship between water activity and fish spoilage during cold storage: A review. J. Food Agric. Environ 2009, 7, 86-90.
33. Jellouli, K.; Balti, R.; Bougatef, A.; Hmidet, N.; Barkia, A.; Nasri, M., Chemical composition and characteristics of skin gelatin from grey triggerfish (Balistes capriscus). LWT-Food Science and Technology 2011, 44, 1965-1970.
34. Balti, R.; Jridi, M.; Sila, A.; Souissi, N.; Nedjar-Arroume, N.; Guillochon, D.; Nasri, M., Extraction and functional properties of gelatin from the skin of cuttlefish (Sepia officinalis) using smooth hound crude acid protease-aided process. Food Hydrocolloids 2011, 25, 943-950.
35. Gmia, G. H., Gelatin Manufacturers Institute of America. New York 2012.
36. Kittiphattanabawon, P.; Benjakul, S.; Visessanguan, W.; Shahidi, F., Comparative study on characteristics of gelatin from the skins of brownbanded bamboo shark and blacktip shark as affected by extraction conditions. Food Hydrocolloids 2010, 24, 164-171.
37. Araghi, M.; Moslehi, Z.; Mohammadi Nafchi, A.; Mostahsan, A.; Salamat, N.; Daraei Garmakhany, A., Cold water fish gelatin modification by a natural phenolic cross-linker (ferulic acid and caffeic acid). Food science & nutrition 2015, 3, 370-375.
38. Rawdkuen, S.; Thitipramote, N.; Benjakul, S., Preparation and functional characterisation of fish skin gelatin and comparison with commercial gelatin. International Journal of Food Science & Technology 2013, 48, 1093-1102.
39. Ninan, G.; Joseph, J.; Aliyamveettil, Z. A., A comparative study on the physical, chemical and functional properties of carp skin and mammalian gelatins. J Food Sci Technol 2014, 51, 2085-2091.
40. Ktari, N.; Jridi, M.; Nasri, R.; Lassoued, I.; Ayed, H. B.; Barkia, A.; Nasri, M., Characteristics and functional properties of gelatin from zebra blenny (Salaria basilisca) skin. LWT-Food Science and Technology 2014, 58, 602-608.
41. Kim, S. K., Marine cosmeceuticals. Journal of cosmetic dermatology 2014, 13, 56-67.
42. Srikanya, A., A Study on Optimization of Fish Protein Hydrolysate Preparation by Enzymatic Hydrolysis from Tilapia Fish Waste Mince. Int. J. Curr. Microbiol. App. Sci 2017, 6, 3220-3229.
43. Yi, J.; De Gobba, C.; Skibsted, L. H.; Otte, J., Angiotensin-I converting enzyme inhibitory and antioxidant activity of bioactive peptides produced by enzymatic hydrolysis of skin from grass carp (Ctenopharyngodon idella). International Journal of Food Properties 2017, 20, 1129-1144.
44. Balti, R.; Bougatef, A.; Sila, A.; Guillochon, D.; Dhulster, P.; Nedjar-Arroume, N., Nine novel angiotensin I-converting enzyme (ACE) inhibitory peptides from cuttlefish (Sepia officinalis) muscle protein hydrolysates and antihypertensive effect of the potent active peptide in spontaneously hypertensive rats. Food chemistry 2015, 170, 519-525.
45. Lafarga, T.; Wilm, M.; Wynne, K.; Hayes, M., Bioactive hydrolysates from bovine blood globulins: Generation, characterisation, and in silico prediction of toxicity and allergenicity. Journal of Functional Foods 2016, 24, 142-155.
46. Minkiewicz, P.; Dziuba, J.; Iwaniak, A.; Dziuba, M.; Darewicz, M., BIOPEP database and other programs for processing bioactive peptide sequences. Journal of AOAC International 2008, 91, 965-980.
47. Fu, Y.; Young, J. F.; Lokke, M. M.; Lametsch, R.; Aluko, R. E.; Therkildsen, M., Revalorisation of bovine collagen as a potential precursor of angiotensin I-converting enzyme (ACE) inhibitory peptides based on in silico and in vitro protein digestions. Journal of Functional Foods 2016, 24, 196-206.

1. A method for producing a marine peptide product from a marine by-product derived from fish processing, the method comprising:
filtering a marine by-product, wherein the marine by-product is (i) blue whiting or mackerel bloodwater, or (ii) one or more of fish skins, fish bone and gut waste from blue whiting or mackerel, using one or more filters selected from 3 kDa filter membrane and 10 kDa filter membrane, to obtain at least one retentate, a 3 kDa fraction and/or a 10 kDa fraction,
wherein the marine peptide product is the obtained fraction(s).

2. The method of claim 1, further comprising a step of drying the at least one retentate, 10 kDa fraction and/or 3 kDa fraction.

3. The method of claim 1 or 2, wherein the by-product is bloodwater from blue whiting or mackerel.

4. The method of claim 1 or 2, wherein the by-product is one or more of fish skins, fish bone, and gut waste from blue whiting or mackerel.

5. The method of claim 4, wherein the by-product is one or more of fish skins, fish bone, and gut waste from blue whiting.

6. The method of claim 4 or 5, wherein the method comprises a step of extracting gelatine from the by-product to provide a by-product in the form of a gelatine extract, prior to filtering.

7. The method of claim 4 to 6, wherein the method comprises a step of hydrolysing the by-product, or the gelatine extract, with one or more hydrolysis enzymes prior to filtering.

8. The method of claim 7, wherein the enzymes are alcalase or papain.

9. A method for producing a marine peptide product from a mackerel by-product derived from fish processing, the method comprising:
hydrolysing the mackerel by-product, which is one or more of fish skins, fish bone and gut waste, with one or more hydrolysis enzymes selected from alcalase and papain,
using one or more filters selected from 3 kDa filter membrane and 10 kDa filter membrane, to obtain at least one retentate, a 3 kDa fraction and/or a 10 kDa fraction,
wherein the marine peptide product is the obtained fraction(s).

10. The method of claim 9, wherein the method comprises an initial step of extracting gelatine from the mackerel by-product prior to hydrolysing to provide a mackerel by-product in the form of a gelatine extract.

11. A marine peptide product obtained from the method of any one of claims 1 to 10.

12. A marine peptide product obtained from the method of any one of claims 4 to 10, wherein the product is the 3 kDa fraction.

13. A marine peptide product obtained from the method of any one of claims 1 to 3, wherein the product is a 10 kDa fraction.

14. A marine peptide product comprising one or more peptides selected from peptides comprising SEQUENCE ID NO. 1 to SEQUENCE ID NO. 64.

15. The marine peptide product of claim 14, comprising all the peptides selected from the peptides comprising SEQUENCE ID NO. 1 to SEQUENCE ID NO. 64.

16. The marine peptide product of claim 14 or 15, wherein the peptides consist of a sequence selected from SEQUENCE ID NO. 1 to SEQUENCE ID NO. 64.

17. A marine peptide product comprising one or more peptides selected from peptides comprising SEQUENCE ID NO. 65 to SEQUENCE ID NO. 169.

18. The marine peptide product of claim 17, comprising all the peptides selected form peptides comprising SEQUENCE ID NO. 65 to SEQUENCE ID NO. 169.

19. The marine peptide product of claim 17 or 18, wherein the peptides consist of a sequence selected from SEQUENCE ID NO. 65 to SEQUENCE ID NO. 169.

20. A marine peptide product comprising one or more peptides selected from peptides comprising SEQUENCE ID NO. 170 to SEQUENCE ID NO. 188.

21. The marine peptide product of claim 20, comprising all the peptides selected from peptides comprising SEQUENCE ID NO. 170 to SEQUENCE ID NO. 188.

22. The marine peptide product of claim 20 or 21, wherein the peptides consist of a sequence selected from SEQUENCE ID NO.170 to SEQUENCE ID NO. 188.

23. A marine peptide product comprising one or more peptides selected from peptides comprising SEQUENCE ID NO. 189 to SEQUENCE ID NO. 271 and SEQUENCE 363.

24. The marine peptide product of claim 23, comprising all the peptides selected from peptides comprising SEQUENCE ID NO. 189 to SEQUENCE ID NO. 271 and SEQUENCE 363.

25. The marine peptide product of claim 23 or 24, wherein the peptides consist of a sequence selected from SEQUENCE ID NO.189 to SEQUENCE ID NO. 271 and SEQUENCE 363.

26. A marine peptide product comprising one or more peptides selected from peptides comprising SEQUENCE ID NO. 272 to SEQUENCE ID NO. 362.

27. The marine peptide product of claim 26, comprising all the peptides selected from peptides comprising SEQUENCE ID NO. 272 to SEQUENCE ID NO. 362.

28. The marine peptide product of claim 26 or 27, wherein the peptides consist of a sequence selected from SEQUENCE ID NO.272 to SEQUENCE ID NO. 362.

30. A peptide comprising a sequence selected from SEQUENCE ID NO. 1 to SEQUENCE ID NO. 363, or an ACE-1 inhibitory variant thereof having 1 to 3 amino acid changes, wherein said the or each amino acid change is selected from an insertion deletion, addition, and substitution.

31. The peptide of claim 30, wherein the peptide comprises a sequence selected from SEQUENCE ID NO. 1 to 188 or an ACE-1 inhibitory variant thereof having 1 to 3 amino acid changes, wherein said the or each change is selected from an insertion, deletion, addition, and substitution.

32. The peptide of claim 31, wherein the peptide consists of a sequence selected from SEQUENCE ID NO. 1 to 188.

33. The peptide of claim 30, wherein the peptide comprises a sequence selected from SEQUENCE ID NO. 1, 2, 10, 13, 16, 22, 25, 32, 44, 57, 58, 64 and 62, or an ACE-1 inhibitory variant thereof having 1 to 3 amino acid changes, wherein said the or each amino acid change is selected from an insertion deletion, addition, and substitution.

34. The peptide of claim 33, wherein the peptide consists of a sequence selected from SEQUENCE ID NO. 1, 2, 10, 13, 16, 22, 25, 32, 44, 57, 58, 64 and 62.

35. The peptide of claim 30, wherein the peptide comprises a sequence selected from SEQUENCE ID NO. 122, 123, 133, 139, 141, 147, 148, 150, 152, 157 and 160 or an ACE-1 inhibitory variant thereof having 1 to 3 amino acid changes, wherein said the or each amino acid change is selected from an insertion deletion, addition, and substitution.

36. The peptide of claim 35, wherein the peptide consists of a sequence selected from SEQUENCE ID NO. 122, 123, 133, 139, 141, 147, 148, 150, 152, 157 and 160.

37. The peptide of claim 30, wherein the peptide comprises a sequence selected from SEQUENCE ID NO 170, 171, 178, 182, 183, 187, 185, 184 and 186 or an ACE-1 inhibitory variant thereof having 1 to 3 amino acid changes, wherein said the or each change is selected from an insertion, deletion, addition, and substitution.

38. The peptide of claim 37, wherein the peptide consists of a sequence selected from SEQUENCE ID NO. 170, 171, 178, 182, 183, 187, 185, 184 and 186.

39. The marine peptide product of any one of claims 14 to 28 further comprising at least one fatty acids.

40. The marine peptide product of claim 39, wherein the at least one fatty acid is one or more of the fatty acids of Table 5.

41. An animal food product comprising the marine peptide product of any one of claims 11 to 28 or 39 to 40 or a peptide of any one of claims 30 to 38.

42. The animal food product of claim 41, wherein the marine peptide product is the product of any one of claims 11 to 28 or 39 to 40.

43. The animal food product of claim 41 or 42, wherein the animal is a companion animal.

44. The animal food product of claim 43, wherein the animal is a cat or a dog.

45. A marine peptide product of any one of claims 11 to 28 or 39 to 40, a peptide of any one of claims 30 to 38, or an animal food product of claim 41 to 44, for use as a medicament in an animal.

46. A marine peptide product of any one of claims 11 to 28 or 39 to 40, a peptide of any one of claims 30 to 38, or an animal food product of claim 41 to 44, for use in a method of treating or preventing a disease or condition selected from the group comprising heart disease, renal disease, inflammatory disease or condition, eye disease, high blood pressure and hypertension in an animal.

47. The marine peptide product, peptide, or animal food product for use of claim 46, wherein the disease or condition is selected from the group comprising heart disease, renal disease, inflammatory disease or condition, eye disease, high blood pressure and hypertension.

48. The marine peptide product, peptide, or animal food product for use of claim 47, wherein the method is for preventing or treating hypertension in an animal.

49. The marine peptide product, peptide, or animal food product for use of claim 48, wherein the animal is an elderly dog.

50. The marine peptide product of claim 12, or an animal food product comprising the marine peptide product of claim 12, for use in a method of preventing or treating hypertension in an elderly dog.
